(19) United States
(12) Patent Application Publication
(10) Pub. No.: US 2008/0233604 A1
(43) Pub. Date: Sep. 25, 2008

(54) Methods For Identifying Compounds Capable of Modulating the Hydrolase Activity of Clca Protein

(76) Inventors: Matti Lepisto, Lund (SE); Krzysztof Pawlowski, Lund (SE)

Correspondence Address: FISH & RICHARDSON P.C., P.O BOX 1022, MINNEAPOLIS, MN, 55440-1022, US

(21) Appl. No.: 10/590,691

(22) PCT Filed: Mar. 3, 2005
(86) PCT No.: PCT/SE05/00316

§ 371 Date: Aug. 25, 2006

(30) Foreign Application Priority Data
Mar. 5, 2004 (SE) 0400564-1

(51) Int. Cl. C12Q 1/34

(52) U.S. Cl. 435/18

(57) ABSTRACT

Methods for identifying compounds capable of modulating the hydrolase activity of a CLCA protein include screening and computer modelling methods. The compounds, including antibodies, may be useful as therapeutic agents to treat a variety of diseases.

### FIELD OF THE INVENTION

This invention relates to methods of screening for modulators of the CLCA family of calcium-activated chloride channels, and to methods of modelling or designing such modulators. These modulators may be used as pharmaceutical agents to treat various diseases.

### BACKGROUND OF THE INVENTION

The CLCA family of calcium-activated chloride channels is also known as the CACC family. This family of proteins mediate a Ca²⁺-activated Cl⁻ conductance in a variety of tissues in a variety of species. The following family members have been cloned:
one porcine protein: pCLCA1
two bovine proteins: bCLCA1, bCLCA2 (also known as Lu-ECAM-1);
five murine proteins: mCLCA1, mCLCA2, mCLCA3 (also known as gob-5), mCLCA4, mCLCA5
four human proteins: hCLCA1 (also known as ICACC1 or hCACC1), hCLCA2 (also known as hCACC3), hCLCA3, hCLCA4 (also known as hCACC2)
two rat proteins: rCLCA1, rCLCA.

The full-length sequences of these CLCA proteins are available from the literature and/or from publicly available sequence databases, as shown below. Where a sequence database identifier is quoted, the world wide web (www) or internet address of the relevant sequence database is as follows: TREMBL (http://us.expasy.org/sprot); SwissProt (http://us.expasy.org/sprot/); NCBI Genbank database (http://www.ncbi.nlm.nih.gov/).
Sus scrofa (porcine) pCLCA1 protein: Gaspar K J et al, Physiol. Genomics (Online), 2000, 3:101-111; TREMBL:Q9TUB5.
Bos taurus (bovine) protein bCLCA1: Cunningham S A et al, J Biol Chem, 1995, 270:31016-31026; SWISSPROT:ECLC_BOVIN.
Bos taurus (bovine) protein bCLCA2: Zhu D Z et al, Proc Natl Acad Sci USA, 1991, 88(21):9568-7; database identifier TREMBL:O18744.
Mus musculus (murine) protein mCLCA1: TREMBL:Q8C324
Mus musculus (murine) protein mCLCA2: TREMBL:Q8C9E1
Mus musculus (murine) protein mCLCA3: Komiya T et al, Biochem Biophys Res Commun, 1999, 255:347-351; TREMBL:Q8R049.
Mus musculus (murine) protein mCLCA4: TREMBL:Q91ZF5.
Mus musculus (murine) protein mCLCA5: TREMBL:Q8BG22.
Homo sapiens. (human) protein CLCA1: Agnel M et al, FEBS Lett, 1999 July, 455(3): 295-301; Gruber A D et al, Genomics, 1998, 54:200-214; TREMBL:O95151.
Homo sapiens (human) protein CLCA2: Gruber A D et al, Am J Physiol, 1999, 276:C1261-C1270; Agnel M et al, FEBS Lett, 1999 July, 455(3): 295-301; TREMBL:Q9UNF7.
Homo sapiens (human) protein CLCA3: Gruber A D et al, Biochim Biophys Acta, 1999, 1444:418-423; TREMBL:Q9Y6N3.
Homo sapiens (human) protein CLCA4: Agnel M et al, FEBS Lett, 1999 July, 455(3): 295-301; TREMBL:Q9UQC9.
Rattus norvegicus (rat) protein rCLCA1: WO2003037927; NCBI:XP_{—}217689.2.
Rattus norvegicus (rat) protein rCLCA: TREMBL:BAD0114.

In addition to the two rat CLCA proteins that have been isolated and sequenced, the following five CLCA protein sequences have been predicted from rat genomic sequences:
a CLCA protein located between residues 1 and 833 of the sequence NCBI:XP_{—}217688.1 (NCBI Genbank database), hereinafter referred to as rCLCA3.
a CLCA protein located between residues 851 and 1776 of the sequence NCBI:XP_{—}217688.1 NCBI Genbank database), hereinafter referred to as rCLCA4.
a CLCA protein located between residues 3691 and 4637 of the sequence NCBI:XP_{—}217688.1 (NCBI Genbank database), hereinafter referred to as rCLCA5.
a CLCA protein hereinafter referred to as rCLCA6: NCBI:XP_{—}217690.2 (NCBI Genbank database).
a CLCA protein hereinafter referred to as rCLCA7: NCBI:XP_{—}342357.1 (NCBI Genbank database).

Equivalent CLCA proteins have been identified in other species, including the tunicate Ciona intestinalis, two fish species and two frog species. Some of these proteins have not been fully sequenced, others are proteins predicted from genomic sequences. It is believed that equivalent CLCA proteins exist in all vertebrates (including mammals).

For example, the following six sequences are predicted full-ength sequences of CLCA proteins in the tunicate Ciona intestinalis (translated from the known sequences of CLCA genes). The sequences are listed in the DOE Ciona (ci) database (http://genome.jgi-psf.org/ciona4/ciona4.home.html) under the sequence identifiers: ci0100131812, ci0100132657, ci0100137033, ci0100140780, ci0100141485, ci0100148238.

All the CLCA protein and nucleic acid sequences cited above are incorporated herein by reference.

The best characterised CLCA family member is bCLCA2. Important structural motifs have been identified in the protein, such as the symmetrical spacing of five cysteine residues in the N-terminal domain which may be involved in disulphide bonds or a motif that could be involved in binding of metal ions (Zn). Other motifs are sites for N-linked glycosylation as well as sites for Ca²⁺/calmodulin kinase II.

All known human CLCA genes are clustered on the short arm of chromosome 1. Except for hCLCA3, which is a truncated and secreted protein, the other human proteins are synthesized as 125 kD precursor transmembrane proteins that are rapidly cleaved to 90 and 35 kD subunits. The 90 kD subunit is believed to be anchored in the plasma membrane via four transmembrane domains. It has been suggested that the 35 kD subunit may be associated with the 90 kD subunit on the outside of the cell membrane.

Two alternative sets of locations of transmembrane regions in CLCA have been proposed on the basis of experiment and simple computational analysis. The presence of a von Willebrand factor type A (VWA) domain in CLCA proteins has been noted by Whittaker and Hynes, M B C, 2002, 13:3369-3387. The von Willebrand factor type A domain is an ubiquitous extracellular protein domain known to be involved in cell adhesion, in extracellular matrix proteins, and in integrin receptors. It is present in more than 500 different proteins. The role of VWA domain in CLCA is currently not clear, but may be related to scaffolding and/or oligomerization of the CLCA molecule and also modulation of channel activity by binding other proteins.

The three dimensional structures of CLCA proteins are not known. No three dimensional structure has been determined experimentally for any CLCA protein. Also, no complete three dimensional structure has been predicted for any CLCA protein.

It is generally believed that CLCA proteins are calcium-activated chloride channels, and there is much evidence to support this role. However it has also been suggested that the CLCA proteins may be modulating proteins that affect the activity of the actual ion channel (another protein).

Each CLCA family member has a distinct, but sometimes overlapping, tissue expression pattern. hCLCA1, hCLCA4, mCLCA1 and mCLCA3 are expressed in intestinal epithelia. hCLCA3, hCLCA2 and mCLCA1 are expressed in respiratory epithelia. hCLCA1, hCLCA4 and mCLCA1 are expressed in uterus, prostate, epididymis and testes. hCLCA1, hCLCA2 and mCLCA1 are expressed in the kidney. hCLCA2, mCLCA1 and mCLCA2 are expressed in mammary epithelium, and hCLCA4 is expressed in the brain.

In the airways, hCLCA2, the truncated hCLCA3 and hCLCA4 are expressed under normal conditions. hCLCA1 is normally expressed mainly in the intestine, but also in the uterus, prostate, epididymis, testis and kidney and not in the lung or airways. However, recent data from both murine animal models and human airway biopsies obtained from asthma and COPD patients demonstrate upregulation of hCLCA1 in the inflamed airway.

Heterologous expression of hCLCA1, hCLCA2 and mCLCA1 in HEK293 cells is associated with a calcium-sensitive chloride conductance. It has been shown that the CLCA proteins are activated by addition of the Ca²⁺ ionophore ionomycin under patch clamp conditions. The current generated can be inhibited by classic chloride channel blockers such as DIDS, tamoxifen and niflumic acid. It has also been shown that IP₄, a is metabolite of the phospholipase C cascade which accumulates in cells after α-adrenergic or cholinergic stimulation, is a potent inhibitor of calcium-mediated chloride secretion in T84 cells and pancreatic duct cells from cystic fibrosis patients. This molecule might be responsible for the transitory nature of Ca²⁺-induced secretory responses in epithelial tissues.

In addition to their anion channel properties, certain CLCA family members seem to serve as cell-adhesion molecules having a role in tumour metastasis and in one case (hCLCA2) a tumor suppressive effect of the protein has been suggested.

The hCLCA1 chloride channel has been suggested as a new therapeutic target, regulating abnormal mucus production and mucosal inflammation. This new therapeutic target is potentially associated with the pathogenesis of a variety of nasal, sinus, and other respiratory disorders including cystic fibrosis, chronic bronchitis, allergic rhinitis, asthma, chronic sinusitis, and COPD (chronic obstructive pulmonary disease). It is also potentially associated with the pathogenesis of a variety of gastrointestinal disorders.

The international patent application published as WO99/44620 describes hCLCA1 as a therapeutic target in IL-9 mediated development of atopic allergy, asthma-related disorders and cystic fibrosis. It also describes methods for identifying inhibitors of the hCLCA1 gene and its products and the use of such inhibitors to treat those disorders. Inhibitors of hCLCA1 were defined as compounds that down-regulate the chloride channel function of hCLCA1 or the expression of hCLCA1. One particular method of screening for hCLCA1 inhibitors was a competitive binding assay with natural ligands of hCLCA1. Another method involved in vitro primary lung cultures that produce secreted eotaxin protein upon IL-9 stimulation. It was suggested that treatment with hCLCA1 inhibitors would result in suppression of IL-9 induced eotaxin response. The application also describes the production of antibodies that specifically bind to hCLCA1 or certain fragments of hCLCA1. Such antibodies may be used to quantify. hCLCA1 or may be used as inhibitors by blocking hCLCA1 chloride. channel activity through binding to extracellular regions of the protein required for ligand binding or activation.

The US patent application published as US2003059434 describes a method of treating a subject having a disease state associated with a mucus secretion disorder of the gastrointestinal tract comprising administering to the subject an effective amount of a chloride channel modulator. In particular, this application describes treating diseases such as inflammatory bowel syndrome, ulcerative colitis and Crohn syndrome with a modulator of the hCLCA1 chloride channel. The application describes a method of screening for a compound that modulates hCLCA1 activity by contacting hCLCA1 or a fragment thereof with the compound and detecting modulation of hCLCA1 activity. Whether a given agent acts as an hCLCA1 modulator can be determined by the following methods:
by functional assays of the hCLCA1 polypeptide, to determine whether its activity as a calcium activated chloride channel is modulated;
by direct measurement of the binding or interaction of the compound with hCLCA1 (including competitive binding assays);
by immunological assays (for example, using an antibody specific for a CLCA1 protein to determine whether protein levels of CLCA1 are affected);
by assays to determine whether gene expression of the CLCA1 is affected;
by assays for mucus production by a mucus-producing cell of the gastrointestinal tract.

Active proteins, such as enzymes, involved in physiological and pathological processes are important targets in the development of pharmaceutical compounds and treatments. Knowledge of the three dimensional (tertiary) structure of active proteins allows the rational design of modulators of such proteins. By searching structural databases of compounds using structural parameters derived from the active protein of interest, it is possible to select compound structures that may interact with these parameters. It is then possible to synthesise the selected compound and test its activity. Alternatively, the structural parameters derived from the active protein of interest may be used to design and synthesise a modulator with the desired activity. Such modulators may be useful as therapeutic agents for treating certain diseases. For example, WO98/07835 discloses crystal structures of a protein tyrosine kinase optionally complexed with one or more compounds. The atomic coordinates of the enzyme structures and any of the bound compounds are used to determine the three dimensional structures of kinases with unknown structure and to identify modulators of kinase functions. As another example, WO99/01476 discloses the crystal structures of anti-Factor DC Fab fragments (antibodies) and their use to identify and design new anticoagulant agents.

The practice of the present invention will employ, unless otherwise indicated, conventional methods of virology, immunology, microbiology, molecular biology and recombinant DNA techniques within the skill of the art. Such techniques are explained fully in the literature. See for example: Sambrook et al. eds., Molecular Cloning: A Laboratory Manual (3^{rd} ed.) Cold Spring Harbor Laboratory Press, Cold Spring Harbor, N.Y. (2001); Ausubel et al., eds., Current Protocols in Molecular Biology, John Wiley & Sons, New York, N.Y. (2002); Glover & Hames, eds., DNA Cloning 3: A Practical Approach, Vols. I, II, & III, IRL Press, Oxford (1995); Colowick & Kaplan, eds., Methods in Enzymology, Academic Press; Weir et al, eds., Handbook of Experimental Immunology, 5^{th} ed., Blackwell Scientific Publications, Ltd., Edinburgh, (1997); Fields, Knipe, & Howley, eds., Fields Virology (3^{rd} ed.) Vols. I & II, Lippincott Williams & Wilkins Pubs. (1996); Flint, et al., eds., Principles of Virology: Molecular Biology, Pathogenesis, and Control, ASM Press, (1999); Coligan et al., eds., Current Protocols in Immunology, John Wiley & Sons, New York, N.Y. (2002).

The practice of the present invention will employ, unless otherwise indicated, conventional methods of molecular modelling. These methods include Sybyl, Maestro, GOLD, Ludi, LeapFrog and Macromodel computer programs with algorithms and modules therein, as well as other 3D-modelling techniques and tools known to those skilled in the art. Such 3D-modelling techniques were reviewed by Lyne P D in Drug Discov Today (2002), 7:1047-55.

### SUMMARY OF THE INVENTION

We have now identified a metal-dependent hydrolase domain in the CLCA family of calcium-activated chloride channels. It was not previously known that CLCA family members possess a hydrolase domain or hydrolase activity.

The hydrolase activity of each CLCA protein is believed to be important, whether the CLCA protein is itself a calcium-activated chloride channel or whether it is a modulating protein acting on an ion channel. The hydrolase domain may be a domain of an ion channel modulating its own activity, or, alternatively, it may be a domain of a modulating protein acting on a distinct ion channel. It is believed that modulation of the hydrolase activity of a CLCA protein will result in modulation of the associated calcium-activated chloride channel activity. For any particular CLCA protein, increased hydrolase activity may correlate with increased chloride channel activity or increased hydrolase activity may correlate with decreased chloride channel activity. For example, for hCLCA1 it is likely that increased hydrolase activity correlates with increased chloride channel activity.

A hydrolase domain is present in the human CLCA family and in the homologous CLCA families of mouse and rat. It is believed that CLCA proteins including the hydrolase domain will be present in every vertebrate species, including all mammals. Mouse, rat, guinea pig, hamster, dog and monkey are commonly used as model organisms when testing or developing pharmaceutical agents for use in humans.

We identified the hydrolase domain by complex bioinformatics analysis of known CLCA proteins, and subsequently validated existence of the hydrolase domain by structural modelling. We have cloned and expressed an hCLCA1 hydrolase domain protein.

Knowledge of the novel hydrolase domain is useful for diagnostic and therapeutic applications, as explained below.

We now provide alternative and improved screening methods for identifying compounds that modulate the activity of a CLCA protein. Such screening methods involve assaying the hydrolase activity of the CLCA protein. Previously known screening methods using functional assays have focussed on measurement of the CLCA chloride channel activity. A disadvantage of the known screening methods is that most anions, including chloride (Cl⁻), are difficult to track. There are emerging methods based on fluorescent ion probes or atomic absorption, but these mainly apply to cations like Ca²⁺, Na⁺ and K⁺. Another disadvantage of the known screening methods is that chloride channel activity can only be measured in whole-cell systems, which increases the complexity of primary screening to identify potential CLCA modulators. Thus the fill exploitation of ion channels as a class of molecular drug targets is hampered by the lack of efficient screening technology. Screening for modulators of the hydrolase activity is advantageous because it does not require primary screen whole cell methodology. The complexity of the assays used in the primary screen is thus minimised. A biochemical enzyme assay allows the use of screening formats that are simple, robust and amenable to high throughput compound testing.

We further provide methods to design small molecule compounds that may interact with the hydrolase domain of a CLCA protein and thus may modulate the hydrolase activity of the CLCA protein. The small molecules are evaluated and optimized by computer modelling of covalent or non-covalent interactions between the small molecules and the CLCA hydrolase domain model. Specific protease modulators targeted at the hydrolase activity of the CLCA protein should be easier to design than specific ion channel modulators. In other words, it should be possible to obtain a better compound faster when targeting a hydrolase as compared to targeting an ion channel directly.

Modulators of CLCA hydrolase activity may be useful as therapeutic agents to treat a variety of diseases.

As defined herein, modulation includes any effect on the hydrolase activity of a CLCA protein. Thus modulation may include, for example, any one or more of the following: conformational change, covalent modification, activation, inhibition. Modulators include activators (such as agonists) and inhibitors (such as antagonists). Modulation may be achieved, for example, by increasing or decreasing enzyme activity per se or by increasing or decreasing the interaction of the CLCA protein with accessory proteins. Modulation of a CLCA protein by a compound may be brought about, for example, through compound binding to the CLCA protein.

CLCA proteins are potential targets for therapeutic intervention in various diseases. It is possible to devise screening methods to identify compounds (chemical or biological) that modulate the hydrolase activity of a CLCA protein (preferably a human CLCA protein, and most preferably hCLCA1). Such compounds (modulators) include, for example, chemical or hormonal therapeutic agents that modulate the protein. Such compounds may prove useful as therapeutic agents in treating various diseases or disorders in humans and/or other animals. In particular, such compounds may prove useful as therapeutic agents in treating any disease or condition in which the increased or decreased hydrolase activity or unregulated hydrolase activity of a CLCA protein is involved.

The screening methods of the invention are useful in determining whether or not test compounds (chemical or biological) may be suitable for use, inter alia, in the treatment of gastrointestinal disorders (for example inflammatory bowel syndrome, ulcerative colitis, Crohn syndrome) or in the treatment of nasal, sinus, and other respiratory diseases or disorders including cystic fibrosis, chronic bronchitis, allergic rhinitis, asthma, chronic sinusitis, and COPD (chronic obstructive pulmonary disease), or in the treatment of cancer. The screening methods of the invention are particularly useful in determining whether or not test compounds (chemical or biological) may be suitable for use in the treatment of respiratory diseases or disorders, particularly asthma or COPD.

Different forms of modulation may be required in the treatment of different diseases. For example, in the treatment of asthma or COPD in humans it may be necessary to inhibit the chloride channel activity of hCLCA1 and this may be achieved by appropriate modulation of hCLCA1 hydrolase activity (most probably by inhibition of hCLCA1 hydrolase activity). As another example, in the treatment of cancer in humans it may be necessary to activate the chloride channel activity of hCLCA2 and this may be achieved by appropriate modulation of hCLCA2 hydrolase activity.

It will be appreciated that the terms “treating” and “treatment of”, and variations thereon, include therapeutic and prophylactic (preventative) treatment. Such treatment may involve humans or other animals (preferably humans) susceptible to or suffering from the various diseases or disorders.

CLCA modulators are preferably administered in suitable pharmaceutical compositions.

The invention further provides a method to design and produce new antibodies that bind specifically to the hydrolase domain of a CLCA protein, including antibodies that bind specifically to substrate binding regions (the active sites) of the hydrolase domain. These antibodies may be useful for diagnostic or for therapeutic purposes. Antibodies to the ligand binding regions of the hydrolase domain may be used for therapeutic modulation of CLCA activity as they block access to the active site for substrates. Using antibodies specific for the hydrolase domain, rather than using any of the known CLCA antibodies, is particularly advantageous in diagnostic methods because it allows detection of the functionally important protein region. Using antibodies specific for ligand binding regions of the hydrolase domain, rather than using any of the known CLCA antibodies, is particularly advantageous in therapeutic methods because such antibodies directly modulate the functionally important hydrolase activity.

### DETAILED DESCRIPTION OF THE INVENTION

In a first aspect of the invention we provide a method for identifying a compound capable of modulating the hydrolase activity of a CLCA protein which method comprises:
(a) subjecting one or more test compounds to a screen comprising at least one protein selected from the group consisting of: a CLCA protein or a fragment thereof; a homologue of a CLCA protein or a fragment thereof; and
(b) measuring the hydrolase activity of the CLCA protein or homologue or fragment; and
(c) comparing the measured hydrolase activity with the hydrolase activity of the CLCA protein or homologue or fragment in the absence of the test compound.

For use in a method of the invention, preferably each CLCA protein is a mammalian CLCA protein, and most preferably each CLCA protein is a human CLCA protein (most particularly hCLCA1).

A CLCA protein has the capability to exhibit hydrolase activity under appropriate conditions. A protein that is a homologue of a CLCA protein, a protein that is a fragment of a CLCA protein, and a protein that is a fragment of a homologue of a CLCA protein are all proteins that retain the capability to exhibit hydrolase activity.

The term “fragment” as used herein refers to a sub-sequence of the full length sequence that contains at least 60 consecutive amino acids and preferably at least 100 of the CLCA sequence or of a CLCA homologue. Most preferably a fragment refers to a sub-sequence of the full length sequence that contains, in increasing order of preference, at least 150, 200, 250 consecutive amino acids of the CLCA sequence or of the CLCA homologue. It is understood that the protein for use in the invention may be both a fragment and a homologue of a CLCA protein.

When a fragment of a CLCA protein or its homologue is used, that fragment encodes the hydrolase domain of the CLCA protein or a fragment thereof. Preferably a fragment encoding the full hydrolase domain is used. In most full-length CLCA proteins, the full hydrolase domain is contained in the region between residues 1 and 350, most usually between residues 1 and 300. The hydrolase active site located between positions corresponding to 156 and 168 in hCLCA1 contains residues that are highly conserved between different CLCA proteins within a single species and between different species. These are the residues corresponding to His156, Glu157, His160, Glu168 in hCLCA1.

A fragment is large enough to contain all the functional and structural motifs necessary for hydrolase activity. For example, a suitable fragment would include the catalytic metal ion site located between residues 156 and 168 of hCLCA1, including residues His156, Glu157, His 160, Glu168 (or corresponding residues from other CLCA proteins). A suitable fragment would also include residues of the structural metal ion binding site between residues 115 and 133, including Cys125, Glu127, His133 of hCLCA1 (or corresponding residues from other CLCA proteins). Preferably, a suitable fragment would include the whole region corresponding to residues 50 to 199 of hCLCA1. More preferably, a suitable fragment would also include the cysteine-rich region of the hydrolase domain, and would thus encompass the sequence corresponding to residues 50 to 262 of hCLCA1, or an even larger fragment that would exhibit desired physicochemical properties (such as good solubility).

Suitable protein sequences for use in a method of the invention are provided as SEQ ID Nos: 1 to 37 in the Sequence Listing provided herein. These sequences are fragments of a CLCA protein encoding the full hydrolase domain of the protein or fragments thereof.

A protein having any one of the following sequences is suitable for use in a screening method of the invention. Each of the following sequences encodes a complete hydrolase domain of a CLCA protein.
SEQ ID NO:1 from Bos taurus: corresponds to residues 8 to 309 of full-length bCLCA2; the hydrolase active site is located between residues 155 and 167 of bCLCA2.
SEQ ID NO:12 from Bos taurus: corresponds to residues 1 to 308 of full-length bCLCA1; the hydrolase active site is located between residues 155 and 167 of bCLCA1.
SEQ ID NO:2 from Homo sapiens: corresponds to residues 1 to 306 of full-length hCLCA1; the hydrolase active site is located between residues 156 and 168 of hCLCA1.
SEQ ID NO:37 from Homo sapiens: corresponds to residues 40 to 201 of full-length hCLCA1; the hydrolase active site is located between residues 156 and 168 of hCLCA1.
SEQ ID NO:3 from Homo sapiens: corresponds to residues 1 to 306 of full-length hCLCA2; the hydrolase active site is located between residues 155 and 167 of hCLCA2.
SEQ ID NO:4 from Homo sapiens: corresponds to residues 8 to 311 of full-length hCLCA4; the hydrolase active site is located between residues 164 and 176 of hCLCA4.
SEQ ID NO:5 from Homo sapiens: corresponds to residues 3 to 261 of full-length hCLCA3; the hydrolase active site is located between residues 155 and 167 of hCLCA3.
SEQ ID NO:6 from Mus musculus: corresponds to residues 33 to 311 of full-length mCLCA5; the hydrolase active site is located between residues 164 and 176 of mCLCA5.
SEQ ID NO:7 from Mus musculus: corresponds to residues 1 to 308 of full-length mCLCA1; the hydrolase active site is located between residues 155 and 167 of mCLCA1.
SEQ ID NO:8 from Mus musculus: corresponds to residues 1 to 308 of full-length mCLCA2; the hydrolase active site is located between residues 155 and 167 of mCLCA2.
SEQ ID NO:9 from Mus musculus: corresponds to residues 1 to 307 of full-length mCLCA3; the hydrolase active site is located between residues 156 and 168 of mCLCA3.
SEQ ID NO:10 from Mus musculus: corresponds to residues 1 to 308 of full-length mCLCA4; the hydrolase active site is located between residues 155 and 167 of mCLCA4.
SEQ ID NO:11 from Sus scrofa: corresponds to residues 1 to 306 of full-length pCLCA1; the hydrolase active site is located between residues 156 and 168 of pCLCA1.
SEQ ID NO:33 from Rattus Norvegicus: corresponds to residues 1-307 of full-length rCLCA1; the hydrolase active site is located between residues 156 and 168 of rCLCA1.
SEQ ID NO:36 from Rattus norvegicus: corresponds to residues 1 to 308 of full-length rCLCA (predicted protein sequence); the hydrolase active site is located between residues 155 and 167 of rCLCA.
SEQ ID NO:30 from Rattus Norvegicus: corresponds to residues 54 to 254 of full-length rCLCA3 (predicted protein sequence, equivalent to residues 54 to 254 of full-length NCBI:XP_{—}217688.1); the hydrolase active site is located between residues 97 and 109 of rCLCA3 (equivalent to residues 97 and 109 of full-length NCBI:XP_{—}217688.1).
SEQ ID NO:31 from Rattus Norvegicus: corresponds to residues 1 to 333 of full length rCLCA4 (predicted protein sequence, equivalent to residues 851 to 1183 of full-length NCBI:XP_{—}217688.1); the hydrolase active site is located between residues 138 and 250 of rCLCA4 (equivalent to residues 988 and 1000 of full-length NCBI:XP_{—}217688.1).
SEQ ID NO:32 from Rattus Norvegicus: corresponds to residues 1 to 335 of rCLCA5 (predicted protein sequence, equivalent to residues 3691 to 4025 of full-length NCBI:XP_{—}217688.1); the hydrolase active site is located between residues 155 and 167 of rCLCA5 (equivalent to residues 3845 and 3857 of full-length NCBI:XP_{—}217688.1).
SEQ ID NO:34 from Rattus Norvegicus: corresponds to residues 33 to 311 of full-length rCLCA6 (predicted protein sequence); the hydrolase active site is located between residues 164 and 176 of rCLCA6.
SEQ ID NO:35 from Rattus Norvegicus: corresponds to residues 2 to 247 of full-length rCLCA7 (predicted protein sequence); the hydrolase active site is located between residues 156 and 168 of rCLCA7.
SEQ ID NO:13 from Ciona intestinalis: corresponds to residues 100 to 346 of full-length ci0100131812 (predicted protein sequence); the hydrolase active site is located between residues 210 and 222 of ci0100131812.
SEQ ID NO:14 from Ciona intestinalis: corresponds to residues 1 to 274 of full-length ci0100132657 (predicted protein sequence); the hydrolase active site is located between residues 117 and 129 of ci0100132657.
SEQ ID NO:15 from Ciona intestinalis: corresponds to residues 1 to 282 of full-length ci0100137033 (predicted protein sequence); the hydrolase active site is located between residues 131 and 143 of ci0100137033.
SEQ ID NO:16 from Ciona intestinalis: corresponds to residues 1 to 286 of full-length ci0100140780 (predicted protein sequence); the hydrolase active site is located between residues 134 and 146 of ci0100140780.
SEQ ID NO:17 from Ciona intestinalis: corresponds to residues 1 to 273 of full-length ci0100141485 (predicted protein sequence); the hydrolase active site is located between residues 133 and 145 of ci0100141485.
SEQ ID NO: 18 from Ciona intestinalis: corresponds to residues 24 to 302 of full-length ci0100148238 (predicted protein sequence); the hydrolase active site is located between residues 159 and 171 of ci0100148238.

A protein having any one of the following sequences is suitable for use in a screening method of the invention. Each of the following sequences encodes a fragment of a hydrolase domain of a CLCA protein. Sequences are translated from cDNA sequences (Expressed Sequence Tag or EST). The publicly available EST databases store nucleic acid sequences which are fragments of the expressed region of a gene. Where a sequence database identifier is quoted, the world wide web (www) or internet address of the relevant EST sequence database is as follows: EMBL Nucleotide database (http://www.ebi.ac.uk/embl/index.html).
SEQ ID NO:19 from Danio rerio (zebrafish), EMBLEST:AW174117 (sequence annotated as similar to bovine CLCA, Lu-ECAM-1).
SEQ ID NO:20 from Gallzis gallus (chicken), EMBLEST:BU122641.
SEQ ID NO:21 from Gallus gallus (chicken), EMBLNEW:CF249701.
SEQ ID NO:22 from Salmo salar (Atlantic salmon), EMBLNEW:CA043044.
SEQ ID NO:23 from Strongylocentrotus purpuratus (sea urchin), EMBLNEW:CD296258.
SEQ ID NO:24 from Strongylocentrotus purpuratus (sea urchin), EMBLNEW:CD306326.
SEQ ID NO:25 from Strongylocentrotus purpuratus (sea urchin), EMBLNEW:CD308947.
SEQ ID NO:26 from Xenopis tropicalis (western clawed frog), EMBLEST:BQ392061.
SEQ ID NO:29 from Xenopus tropicalis (western clawed frog), EMBLEST:AL972392.
SEQ ID NO:27 from Xenopus laevis (African clawed frog), EMBLEST:BG018962 (sequence annotated as similar to bovine CLCA, Lu-ECAM-1).
SEQ ID NO:28 from Xenopus laevis (African clawed frog), EMBLNEW:CF286706.

A homologue of a CLCA protein is any variant or isotype of a CLCA protein (including amino acid sequence variants such as alternative splice forms, SNP variants etc). Preferably the homologue used is a mammalian homologue. Preferably each homologue is a protein containing an amino acid sequence possessing, in increasing order of preference, at least 40%, 50%, 60%, 70%, 75%, 80%, 85%, 90%, 95%, 97%, 98% and 99% amino acid sequence identity to a CLCA protein. The sequence identity between two sequences can be determined by pair-wise computer alignment analysis, using programs such as, BestFit, Gap or FrameAlign. The preferred alignment tool is BestFit. In practice, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavor to closely approximate the “gold-standard” alignment algorithm of Smith-Waterman. Thus, the preferred algorithm for use in assessing similarity, i.e. how two primary polypeptide sequences line up, is Smith-Waterman. Identity refers to direct matches, similarity allows for conservative substitutions.

The CLCA protein(s) used in the screening methods of the invention can be prepared by various techniques known to the person skilled in the art. CLCA can be extracted from biological tissue or biological fluids. RNA transcripts can be used to prepare a protein by in vitro translation techniques according to known methods (Sambrook et al. supra). Alternatively, the CLCA protein(s) can be synthesised chemically. For example, by the Merryfield technique (J. Amer. Chem. Soc. 85:2149-2154, (1968)). Numerous automated polypeptide synthesisers, such as Applied Biosystems 431A Peptide Synthesizer also now exist. Alternatively the CLCA protein(s) are produced from a nucleotide sequence encoding the protein using recombinant expression technology. A variety of expression vector/host systems may be used to express the CLCA coding sequences. These include, but are not limited to microorganisms such as bacteria transformed with plasmids, cosmids or bacteriophage; yeasts transformed with expression vectors; insect cell systems transfected with recombinant baculovirus; plant cell systems transfected with plant virus expression systems, such as cauliflower mosaic virus; or mammalian cell systems transfected with plasmids or transduced with recombinant virus (for example adenovirus); selection of the most appropriate system is a matter of choice. Preferably, the CLCA hydrolase domain protein is expressed in bacterial cells, especially E. coli, or in mammalian cells. Mammalian cells provide post-translational modifications to recombinant CLCA protein, which include phosphorylation and glycosylation.

In particular embodiments of a screening method according to the invention, the CLCA protein or homologue or fragment is fused to another peptide or protein sequence to form a fusion protein. In any expression system, the CLCA protein or homologue or a fragment thereof may be expressed as a fusion protein. Such fusion proteins are useful for the detection of expressed protein, facilitating the purification of the protein and/or for increasing the solubility of the protein. When a protein domain or part of a protein is expressed, a fusion protein may increase the solubility and decrease aggregation by interacting with hydrophobic surface-exposed regions of the domain. Examples of such fusion peptides/proteins are poly-histidine, FLAG-, cmyc-, strep-, GST-, MBP-, and GFP-tags. The tag may be fused to the N- or C-terminus of the CLCA protein, or incorporated at a certain position between two amino acid residues of the CLCA sequence.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the protein is placed under the control of an appropriate promoter, control elements and transcription terminator so that the nucleic acid sequence encoding the protein is transcribed into RNA in the host cell transformed or transfected by the expression vector construct. The coding sequence may or may not contain a signal peptide or leader sequence for secretion of the protein out of the host cell. Expression and purification of the CLCA protein(s) can be easily performed using methods well known in the art (for example as described in Sambrook et al. supra).

The methods according to the invention are screening methods and may be operated using conventional procedures. The test compound or compounds to be screened are brought into contact with the purified or partially purified protein(s), or a cell capable of producing it, or a cell membrane preparation or a cell lysate preparation thereof, and modulation of the protein is determined. The conditions of the screen are suitably selected to allow a binding interaction between an active compound (modulator) and the protein. Cells capable of producing the protein include cells naturally expressing CLCA and cells expressing recombinant CLCA.

The screening method of the invention may comprise an assay system wherein the test compound is brought into contact with the purified or partially purified CLCA protein (or a homologue thereof or a fragment of either), and modulation of the protein (or homologue or fragment) is determined. In particular embodiments, the CLCA protein or homologue or fragment is present as a fusion protein. The modulation is determined by measuring modulation of hydrolase activity of CLCA. Methods to measure hydrolase activity are described in the literature and well-known to those skilled in the art. Methods include but are not limited to the following protease assay formats:
Fluorescence intensity using fluorogenic quenched FRET peptide/protein substrates;
Absorbance using chromogenic peptide/protein substrates;
Radioactive formats like SPA or FlashPlate using radioactively labelled biotinylated peptide/protein substrates;
Fluorescence polarization, using fluorescence labelled biotinylated peptide substrates;
AlphaScreen, using biotinylated and tagged (such 6×His, FLAG) protein or peptide substrates;
Label free detection, using LC-MS to demonstrate the cleavage of a peptide/protein substrate;
Label free detection, using SDS-PAGE to demonstrate cleavage of a protein substrate.

Preferably, hydrolase activity is measured by following the hydrolytic cleavage of a fluorogenic or chromogenic peptide or protein substrate.

To measure the hydrolase activity of a CLCA protein, a suitable protein or peptide substrate must first be selected. The substrate may be selected by following standard procedures well-known in the art, including for example by screening of combinatorial peptide libraries (J. Combin. Chem. 2(5), 461-466, (2000); WO 97/40065), by structure-based design (US2002/0151028), or by combinations thereof resulting in mini-libraries/focused libraries (J. Peptide Res. 54, 444-448, (1999); Anal. Biochem. 255, 59-65 (1998)). The structure-based design of substrates is based on the predicted three-dimensional structure of the CLCA hydrolase domain as provided herein and computer molecular modelling methods and an initial di-peptidic substrate model (substructure S in scheme x). The initial di-peptidic substrate is preferably a model where the scissile amide is modelled as the tetrahedral intermediate of a Gly peptide (substructure I in scheme x,).

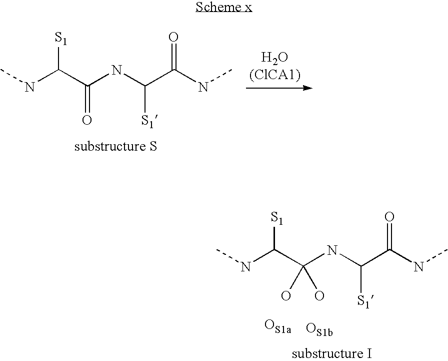

Optionally, Gly di, tri, tetra, penta or hexapeptides are used as initial substrate models as their tetrahedral intermediates regarding the scissile bond (J. Biomol. Structure and Dynamics 17(6), 933-946 (2000)). Side-chains, additional amino acid residues, chromophoric or fluorogenic residues can be added, evaluated and optimized by computer modelling of covalent or non-covalent interactions between the substrate or its intermediate and the CLCA hydrolase domain model. Computer modelling methods include, but are not limited to, Sybyl, Maestro, GOLD, Ludi, LeapFrog and Macromodel computer programs with algorithms and modules therein. Interactions that may be evaluated include, but are not limited to, bond stretching, angle bending, rotational and torsional strain, van der Waals forces, solvation energies, electrostatic and dipole-dipole, charge-dipole and hydrogen bond interactions. Preferred interactions between the initial substrate and enzyme models include, but are not limited to, between O_{S1a} (as defined in scheme x) and Glu157 of hCLCA1 (or corresponding glutamate residue in other CLCA homologs) and O_{S1b} and catalytic metal ion in CLCAs. The peptide substrates thus designed and evaluated are then synthesized as libraries by methods well known to the person skilled in the art. These substrate libraries are next screened to select the most preferred substrates for the modulator screening assays of the invention.

The screening methods of the invention may comprise an assay system wherein the test compound is brought into contact with a cell capable of producing the CLCA protein (or a homologue thereof or a fragment or either), or with a cell membrane preparation thereof, or with a cell lysate preparation thereof and modulation of the CLCA protein (or homologue or fragment) is determined. In particular embodiments, the CLCA protein or homologue or fragment is present as a fusion protein. The modulation is determined by measuring modulation of hydrolase activity of CLCA as described above.

As described herein, cells (including mammalian cells, bacterial cells, yeast cells, insect cells etc) can be engineered to express a CLCA protein. The screening methods of the invention may use a cell or cell line expressing genomic DNA or cDNA encoding a CLCA protein or a homologue thereof, or a fragment of either.

Convenient DNA sequences for use in the various aspects of the invention may be obtained using conventional molecular biology procedures, for example by probing a human genomic or cDNA library with one or more labeled oligonucleotide probes containing 10 or more contiguous nucleotides designed using known CLCA nucleotide sequences. Alternatively, pairs of oligonucleotides one of which is homologous to the sense strand and one to the antisense strand, designed using the nucleotide sequences described here to flank a specific region of DNA may be used to amplify that DNA from a cDNA library. Primers or probes may be manufactured using any convenient method of synthesis. Examples of such methods may be found in standard textbooks, for example “Protocols for Oligonucleotides and Analogues; Synthesis and Properties”, Methods in Molecular Biology Series; Volume 20; Ed. Sudhir Agrawal, Humana ISBN: 0-89603-247-7 (1993); 1^{st} Edition. If required the primer(s) may be labeled to facilitate detection.

Preferably the genomic DNA or cDNA expressing a CLCA protein is a mammalian sequence, and most preferably a human sequence (particularly hCLCA1).

A homologue of a genomic DNA or cDNA expressing a CLCA protein is any DNA variant that encodes a CLCA protein. Preferably each homologue contains a nucleic acid sequence possessing, in increasing order of preference, at least 60%, 70%, 75%, 80%, 85%, 90%, 95%, 97%, 98% and 99% sequence identity to the genomic DNA or cDNA. A fragment of a genomic DNA or cDNA expressing a CLCA protein, or a fragment of a DNA homologue, is a subsequence of the full length sequence that contains at least 10 consecutive bases of the CLCA DNA sequence or of the CLCA DNA homologue. It is understood that the DNA for use in the invention may be both a fragment and a homologue of a CLCA genomic DNA or cDNA.

Any convenient test compound or library of test compounds may be used in conjunction with the screening methods of the invention. Particular test compounds include low molecular weight chemical compounds (preferably with a molecular weight less than 1500 Daltons) suitable as pharmaceutical or veterinary agents for human or animal use, or compounds for non-administered use such as cleaning/sterilizing agents or for agricultural use. Test compounds may also be biological in nature, such as hormones or antibodies. As used herein the term antibody includes both monoclonal, polyclonal, humanized and chimeric antibodies and is to be understood to mean a whole antibody or a fragment thereof, a single chain antibody, a multimeric monospecific antibody or fragment thereof, or a bi- or multi-specific antibody or fragment thereof. Each of these types of antibody and derivative are well known to the person skilled in the art. Methods of making and detecting antibodies are well known (Campbell; Monoclonal Antibody Technology, in: Laboratory Techniques in Biochemistry and Molecular Biology, Volume 13. Eds: Burdon R et al. Elsevier, Amsterdam (1984)).

Any compound identified by any screening method of the invention is selected by the screen as a compound capable of modulating the hydrolase activity of a CLCA protein. Such a compound may prove useful, for example, in treating any disease or condition in which the increased or decreased hydrolase activity or unregulated hydrolase activity of a CLCA protein is involved (for example through its effect on the chloride channel activity). In particular, any compound identified by the screening methods of the invention may prove useful in treating gastrointestinal disorders (for example inflammatory bowel syndrome, ulcerative colitis, Crohn syndrome) or in the treatment of nasal, sinus, and other respiratory diseases or disorders including cystic fibrosis, chronic bronchitis, allergic rhinitis, asthma, chronic sinusitis, and COPD (chronic obstructive pulmonary disease) or in the treatment of cancer. Compounds identified by the screening methods of the invention may be particularly useful in treating respiratory diseases or disorders, particularly asthma or COPD. The invention thus extends to a compound identified by a screening method of the invention as defined herein.

In a further aspect of the invention we provide a compound capable of modulating the hydrolase activity of a CLCA protein, or a pharmaceutically acceptable derivative of the compound, wherein said compound is identified by a screening method of the invention.

The compound may modulate CLCA hydrolase activity by activation or by inhibition. A pharmaceutically acceptable derivative includes a pharmaceutically acceptable salt or ester of the compound.

In a further aspect, we provide use of the compound according to the invention as a therapeutic agent. Such a therapeutic agent may be useful for the treatment of any one of the diseases or disorders discussed above. In a preferred embodiment, the compound is suitable for use in the treatment of respiratory diseases or disorders, particularly asthma or COPD.

In a further aspect of the invention, we provide use of a compound capable of modulating the hydrolase activity of CLCA, or a pharmaceutically acceptable derivative of the compound, in the preparation of a medicament for the treatment of a disease or disorder, wherein said compound is identified by a screening method of the invention.

In a further aspect of the invention we provide a pharmaceutical composition comprising a pharmaceutically acceptable carrier and a compound capable of modulating the hydrolase activity of CLCA, or a pharmaceutically acceptable derivative of the compound, wherein said compound is identified by a screening method of the invention.

A pharmaceutically acceptable carrier may be an excipient or a diluent.

We also provide a method of preparing a pharmaceutical composition which comprises:
i) identifying a compound capable of modulating the hydrolase activity of a CLCA protein, wherein said compound is identified by a screening method of the invention;
ii) mixing the compound or a pharmaceutically acceptable derivative thereof with a pharmaceutically acceptable carrier.

We provide use of any composition according to the invention as a therapeutic agent. Such a therapeutic agent may be useful for the treatment of any one of the diseases or disorders discussed above. In a preferred embodiment, the composition is suitable for use in the treatment of respiratory diseases or disorders, particularly asthma or COPD.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate), anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about S to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

In a further aspect of the invention we provide a method for the treatment of a disease or disorder which comprises administering a therapeutically effective amount of a compound or a pharmaceutically acceptable derivative thereof to a human or other animal, wherein the compound has the capability to modulate the hydrolase activity of a CLCA protein and said compound is identified by a screening method of the invention.

In a further aspect of the invention we provide a method for the treatment of a disease or disorder which comprises administering a therapeutically effective amount of a pharmaceutical composition to a human or other animal, in which the pharmaceutical composition comprises a pharmaceutically acceptable carrier and a compound capable of modulating the hydrolase activity of CLCA, or a pharmaceutically acceptable derivative of the compound, wherein said compound is identified by a screening method of the invention.

According to a further aspect of the invention, we provide methods to design or select chemical modulators of a CLCA protein by using a model of the hydrolase domain structure of a CLCA protein or a homologue thereof or a fragment of either. Small-molecule modulators of a CLCA protein may be designed or selected to fit into the shape of the hydrolase domain region, particularly into the shape of the active site (substrate binding site or cleft).

A modulator of CLCA hydrolase activity may be designed by rational design methods based on interaction of a potential modulator with a CLCA hydrolase domain structure. A modulator of CLCA hydrolase activity may be selected by searching a structural database of compounds using parameters derived from the structure of the CLCA hydrolase domain, and selecting a compound structure that may interact with these parameters. It is then possible to synthesise the designed or selected compound and test its ability to modulate CLCA hydrolase activity.

We provide methods to design or select small molecule compounds that may interact with the hydrolase domain of a CLCA protein and thus may modulate the hydrolase activity of the CLCA protein. The small molecules are evaluated and optimized by computer modelling of covalent or non-covalent interactions between the small molecules and the CLCA hydrolase domain model. Interactions that may be evaluated include bond stretching, angle bending, rotational and torsional strain, van der Waals forces, solvation energies, electrostatic and dipole-dipole, charge-dipole, hydrogen bond, and other relevant interactions. Preferred interactions between the small molecules and enzyme models include a functionality capable of coordinating metal ions such as the catalytic metal ion in CLCA proteins. Suitable modelling methods are known to those skilled in the art. For example, for a review of coordinators used for MMP inhibitors, see Inflammation Research (2003), 52(3), 95-100 and Expert Opinion on Therapeutic Patents (2002), 12(5), 665-707.

A full-atom three-dimensional model of the hydrolase domain of a CLCA protein is defined by the set of atomic coordinates shown in Table 1. To obtain these coordinates, the protein fragment encoded by residues 40 to 201 of the hClCA1 sequence (SEQ ID NO:37) was manually aligned on top of the hMMP-11 structure (PDB code 1hv5) and optimised using standard modules of the Insight II software package (Accelerys Inc.). The resulting model contained the predicted two metal coordinating sequences: 115-133 (‘structural Zn-site’) and 156-168 (‘catalytic Zn-site’). The active site is believed to comprise the amino acid residues within 15 Å of atom Zn-1300 in the set of atomic coordinates shown in Table 1 (found after the Examples).

In Table 1, the amino acid sequence of residues 40 to 201 of hCLCA1 (SEQ ID NO:37) is shown in the lines that begin with the code SEQRES followed by the line number (162 amino acid residues in total). In Table 1 the atomic coordinates are listed in those lines that begin with the code ATOM or HETATM, one atom per line. Following the code are: the unique atom number; the atom name; the amino acid residue name; the amino acid residue number; the atomic coordinates x, y, and z in orthogonal Angstrom space; the atomic occupancy factor (default value for in silico molecular model); the calculated electrostatic charge. Amino acids are abbreviated by three letter codes:
A=ALA=alanine
C=CYS=cysteine
D=ASP=aspartate
E=GLU=glutamate
F=PHE=phenylalanine
G=GLY=glycine
H=HIS=histidine
I=ILE=isoleucine
K=LYS=lysine
L=LEU=leucine
M=MET=methionine
N=ASN=asparagine
P=PRO=proline
Q=GLN=glutamine
R=ARG=arginine
S=SER=serine
T=THR=threonine
V=VAL=valine
W=TRP=tryptophan
Y=TYR=tyrosine.

According to a further aspect of the invention, we provide a method to design a compound capable of modulating CLCA hydrolase activity which comprises molecular modelling based on the interaction of a potential modulator with a hydrolase domain of a CLCA protein or homologue or fragment of either, wherein the three-dimensional structure of the hydrolase domain is defined by the set of atomic coordinates shown in Table 1.

We further provide a method to design a compound capable of modulating CLCA hydrolase activity which comprises molecular modelling based on the interaction of a potential modulator with the active site of a hydrolase domain of a CLCA protein or homologue or fragment of either, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1.

According to a further aspect of the invention, we provide a method for in silico screening for a compound capable of modulating CLCA hydrolase activity which comprises
a) searching a structural database of compounds; and
b) selecting a compound structure that may interact with a hydrolase domain of a CLCA protein or homologue or fragment of either, wherein the three-dimensional structure of the hydrolase domain is defined by the set of atomic coordinates shown in Table 1.

We further provide a method for in silico screening for a compound capable of modulating CLCA hydrolase activity which comprises
a) searching a structural database of compounds; and
b) selecting a compound structure that may interact with the active site of a hydrolase domain of a CLCA protein or homologue or fragment of either, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1.

We further provide uses of therapeutic agents wherein each therapeutic agent is capable of binding to the hydrolase domain of a CLCA protein or homologue thereof or a fragment of either. Preferably the therapeutic agent is selected from the group consisting of: monoclonal antibodies, polyclonal antibodies, humanized antibodies, phage display antibodies, aptamers, constrained peptides, therapeutic peptides, tagged peptides.

Antibodies specifically binding to the hydrolase domain can be designed using the predicted hydrolase domain structure and produced as described below. The predicted three-dimensional structure of the CLCA hydrolase domain can be used to select surface peptide sequences suitable as epitopes for antibody production to enhance the probability of obtaining desired properties of such antibodies. For example, a sequence close to the catalytic cleft (for example hClCA1 sequences Pro117-Gly129, Trp163-Glu173 and Leu177-Arg186) should inhibit the hydrolase activity for therapeutic use. Another example is identification of surface sequences with maximal and inter-species homology (human vs rodents, dog) as diagnostic tools or tools useful in the development of modulators to the hydrolase domain. Yet another example is to select surface sequences which include potential glycosylation sites in order to probe the glycosylation state of the full protein, useful for diagnostic purposes and for development of expression methods for protein production. Such sequences are 5 to 25 amino acids in length, preferably 10 to 20, and located in non-helical regions. The most preferred sequences are soluble and slightly hydrophobic, with calculated logP at −2 to 4, preferably 0 to 2. The sequences can preferably attain the same conformation in solution as they present on the protein surface. The conformational preferences of such peptides can be assessed by computational simulation methods such as molecular dynamics. Such simulations are also useful in distinguishing whether the potential epitope peptide should have free charged N,C-termini or be capped. For a review on structure-guided epitope selection, see Protein Science (1994 October), 3(10), 1670-86.

According to a further aspect of the invention, we provide a method for designing an antibody capable of modulating the hydrolase activity of a CLCA protein which method comprises using the three-dimensional structure of a CLCA hydrolase domain to identify suitable epitopes in the vicinity of the active site, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1. In a particular embodiment of this method, the epitopes include only surface residues within 15 Å of atom Zn-1300 in the set of atomic coordinates shown in Table 1.

Antibodies specifically binding to the hydrolase domain can be raised by introducing the protein domain itself, peptides thereof or genetic material coding for the hydrolase domain or parts thereof into animals or plants. These organisms can be natural breeds or transgenic. Using known antibody generating techniques, antibodies specific towards the hydrolase domain can be raised. Polyclonal and utilising hybridoma technology also monoclonal antibodies can be produced. Antibodies can also be produced by phage display or ribosomal display technologies. These methods can also be combined with affinity maturation techniques and techniques for producing recombinant or engineered antibodies. Covalent display is yet another technology which can be used for antibody production. Production of the antibodies will employ, unless otherwise indicated, conventional methods within the skill of the art. Such techniques are explained fully in the literature. See for example: Handbook of Experimental Immunology. Volume 1: Immunochemistry, Ed by D. M. Weir, Blackwell Scientific Publications, 1986, page 8.1-8.21; Immunotechnology. Ed by J. P. Gosling and D. J. Reen. Portland Press 1993, page 1-11; J. Lipid Research. S.-C. J. Yeung, J. Anderson, K. Kobayashi, K. Oka and L. Chan (1997), 38: 2627-2632; J. Immunol. Meth. S. Nagata, G. Salvatore and I. Pastan (2003), 280: 59-72; Expert Opin. Biol. Ther. G. Nölke, R. Fisher and S. Schillberg (2003), 3(7): 1153-1162; Drug Discovery Today. J. Osburn, L. Jermutus and A. Duncan (2003), 8(18): 845-851; Placenta U. Schmitz, A. Versmold, P. Kaufmann and H.-G. Frank (2000), 21 (suppl. A): S106-S112; J. Immunol. Meth. R. A. Irving, G. Coia, A. Roberts, S. D. Huttall and P. J. Hudson (2001) 248: 31-45; Ann. Rev. Biomed. Eng. J. Maynard and G. Georgiou (2000) 02: 339-376; BioTechniques J. V. Gavilondo and J. W. Larrick (2000), 29(1):128-145.

The present invention will now be described with reference to the following non-limiting Examples.

### EXAMPLE 1

### Expression and Characterisation of an hCLCA1 Hydrolase Domain Protein

The predicted 3-dimensional structure of the hCLCA1 hydrolase domain was used to determine suitable start and end residues of protein fragments that would be expressed as soluble and stable proteins. The sequence close to the N-terminus (Gln52-Met56) threads under a loop (Lys86-Leu105) where a free amino terminus is likely to induce instability. Since the preceding seq. Glu45-Gln51 is predicted to comprise a β-sheet starting with a Pro-x-x-Pro turn, a position preceding the first proline was judged to be a suitable N-terminus for expression. Close to the C-terminus, there is a hydrophobic surface patch that could potentially affect solubility and aggregation. It is therefore advantageous to include an additional 60-100 residues of unpredicted structure, denoted ‘the Cys-rich region’ in the bioinformatics analysis, to occlude the predicted hydrophobic surface. Also, the sequence of the ‘Cys-rich region’ is highly conserved in CLCA variants from different species, which indicates it being part of the hydrolase domain.

Five constructs were made, encoding the following residues of full-length hCLCA1 protein: 50 to 199, 23 to 199, 23 to 63, 45 to 199 and 45 to 263.

The hCLCA1 sequence encoding residues 50-199, 23-199, 23-263, 45-199 and 45-263 was PCR amplified.

Primers for the 50-199 construct were as follows:

| | |
|---|---|
| ATGTCGACCATATGATTCAACAAATAAAGGA | (SEQ ID NO:38) |
| and | |
| ATGCGGCCGCTCACTTCTTTACTACATTTGTAC. | (SEQ ID NO:39) |

Primers for the other constructs were

| | |
|---|---|
| 5′ primer for start at residue 23: | |
| CATATGTCACTCATTCAGCTGAACAAC, | (SEQ ID NO:40) |
| 5′ primer for start at residue 45: | |
| CATATGGAAGATGAAACACTCATTC, | (SEQ ID NO:41) |
| 3′ primer for stop at residue 199: | |
| GCGGCCGCTCACTTCTTTACTACATTTGTACC, | (SEQ ID NO:42) |
| 3′ primer for stop at residue 263: | |
| GCGGCCGCTCACTTGTTTGGAGCTTCTTTG. | (SEQ ID NO:43) |

The sequences of the above primers are included in the Sequence Listing provided herein.

A plasmid containing the full length hCLCA1 sequence was used as template. The PCR fragments were cloned into TA vectors, the correct sequences were verified and the fragments were subcloned into an E. coli expression vector, and inserted into an expression host strain. The proteins were expressed as insoluble inclusion bodies by growing the E. coli expression strain to an OD₆₀₀ of 3-4 and inducing with IPTG for 4-5 h. The cells were harvested, lysed, and the insoluble part of the lysate was separated by centrifugation. The pellets containing the inclusion bodies were solubilised in urea and refolded by a gradual lowering of urea concentration using dialysis. SDS-PAGE of the refolded protein comprising residues 50-199 confirmed the presence of soluble protein of the expected molecular weight 17 kDa. The identity and correct N-terminus of the protein was verified by N-terminal sequencing.

Each of the five hCLCA1 constructs expressed a protein that refolded which indicated that each construct encoded a structural domain of the hCLCA1 protein.

### EXAMPLE 2

### Assaying Hydrolase Activity of hCLCA1 Protein and Screening for Modulators

An in vitro hydrolase assay is used to measure the activity of the refolded hCLCA1 protein fragment produced by the method described in Example 1.

The hydrolase assay measures the hydrolytic cleavage of fluorogenic peptide substrates. Suitable peptide substrates are first identified by design and screening of peptide libraries.

The hydrolase assay is performed in white 384-well plates with each well containing 100 mM Tris-HCl (pH 7.5), 100 mM NaCl, 20 mM CaCl₂, 20 μM ZnCl₂, 0.05% Brij 35, 50 μM fluorogenic substrate and 100 ng of hCLCA1 in a total volume of 80 μl. The assay plates are incubated at room temperature followed by reading in a Tecan Safire at the required time intervals to obtain rates of reaction.

When screening for modulators of hCLCA1 hydrolase activity, the potential modulators are added to dry wells in 1 μl of DMSO giving a final DMSO concentration of 1.25% in the hydrolase assay.

### EXAMPLE 3

### Assaying Hydrolase Activity of hCLCA1 Protein and Screening for Modulators

The purified hClCA1 hydrolase domain (50 ng/ml final concentration) is incubated for 30 minutes at RT in assay buffer (0.1M Tris-HCl, pH 7.3 containing 0.1M NaCl, 20 mM CaCl₂, 0.040 mM ZnCl and 0.05% (w/v) Brij 35) in the presence or absence of inhibitors using the synthetic substrate Mca-Lys-Ala-Met-His-Dpa-OH (SEQ ID NO:44 in the Sequence Listing provided herein). The synthetic substrate contains a modified amino acid (Dpa, (2,4-dinitrophenyl)-L-2,3-diaminopropionyl) and a fluorophore (Mca, (7-methoxy-coumarin-4-yl)acetyl).

Activity is determined by measuring the fluorescence at λex 328 nm and λem 393 nm. Percent inhibition is calculated as follows: % Inhibition is equal to the [Fluorescence_{plus inhibitor}−Fluorescence_{backgrund}] divided by the [Fluorescence_{minus inhibitor}−Fluorescence_{background}].

A similar protocol is used for other expressed and purified CLCA hydrolase domains using substrates and buffers conditions optimal for the particular CLCA, for instance as described for MMPs in C. Graham Knight et al., (1992) FEBS Lett. 296(3):263-266.

TABLE 1

| | | |
|---|---|---|
| SEQRES | 1 | ASP PRO ASN VAL PRO GLU ASP GLU THR LEU ILE GLN GLN |
| SEQRES | 2 | ILE LYS ASP MET VAL THR GLN ALA SER LEU TYR LEU PHE |
| SEQRES | 3 | GLU ALA THR GLY LYS ARG PHE TYR PHE LYS ASN VAL ALA |
| SEQRES | 4 | ILE LEU ILE PRO GLU THR TRP LYS THR LYS ALA ASP TYR |
| SEQRES | 5 | VAL ARG PRO LYS LEU GLU THR TYR LYS ASN ALA ASP VAL |
| SEQRES | 6 | LEU VAL ALA GLU SER THR PRO PRO GLY ASN ASP GLU PRO |
| SEQRES | 7 | TYR THR GLU GLN MET GLY ASN CYS GLY GLU LYS GLY GLU |
| SEQRES | 8 | ARG ILE HIS LEU THR PRO ASP PHE ILE ALA GLY LYS LYS |
| SEQRES | 9 | LEU ALA GLU TYR GLY PRO GLN GLY LYS ALA PHE VAL HIS |
| SEQRES | 10 | GLU TRP ALA HIS LEU ARG TRP GLY VAL PHE ASP GLU TYR |
| SEQRES | 11 | ASN ASN ASP GLU LYS PHE TYR LEU SER ASN GLY ARG ILE |
| SEQRES | 12 | GLN ALA VAL ARG CYS SER ALA GLY ILE THR GLY THR ASN |
| SEQRES | 13 | VAL VAL LYS LYS CYS GLN |

| | | | | | | | | | |
|---|---|---|---|---|---|---|---|---|---|
| ATOM | 1 | N | ASP | 40 | 4.369 | −19.407 | 16.905 | 1.00 | −0.99 |
| ATOM | 2 | CA | ASP | 40 | 4.984 | −18.183 | 17.527 | 1.00 | 0.33 |
| ATOM | 3 | C | ASP | 40 | 3.866 | −17.128 | 17.724 | 1.00 | 0.57 |
| ATOM | 4 | O | ASP | 40 | 3.494 | −16.828 | 18.869 | 1.00 | −0.57 |
| ATOM | 5 | CB | ASP | 40 | 6.271 | −17.685 | 16.869 | 1.00 | −0.11 |
| ATOM | 6 | CG | ASP | 40 | 7.362 | −18.755 | 16.736 | 1.00 | 0.91 |
| ATOM | 7 | OD1 | ASP | 40 | 6.971 | −19.962 | 16.831 | 1.00 | −0.90 |
| ATOM | 8 | OD2 | ASP | 40 | 8.533 | −18.346 | 16.505 | 1.00 | −0.90 |
| ATOM | 9 | N | PRO | 41 | 3.108 | −16.686 | 16.657 | 1.00 | −0.66 |
| ATOM | 10 | CA | PRO | 41 | 2.050 | −15.682 | 16.840 | 1.00 | 0.36 |
| ATOM | 11 | C | PRO | 41 | 0.714 | −16.285 | 17.369 | 1.00 | 0.57 |
| ATOM | 12 | O | PRO | 41 | −0.360 | −15.688 | 17.332 | 1.00 | −0.57 |
| ATOM | 13 | GB | PRO | 41 | 1.859 | −15.087 | 15.446 | 1.00 | 0.00 |
| ATOM | 14 | CG | PRO | 41 | 2.199 | −16.245 | 14.515 | 1.00 | 0.00 |
| ATOM | 15 | CD | PRO | 41 | 3.287 | −17.017 | 15.255 | 1.00 | 0.30 |
| ATOM | 16 | N | ASN | 42 | 0.837 | −17.531 | 17.949 | 1.00 | −0.73 |
| ATOM | 17 | CA | ASN | 42 | −0.230 | −18.189 | 18.691 | 1.00 | 0.36 |
| ATOM | 18 | C | ASN | 42 | −0.121 | −17.919 | 20.213 | 1.00 | 0.57 |
| ATOM | 19 | O | ASN | 42 | −0.985 | −18.305 | 21.003 | 1.00 | −0.57 |
| ATOM | 20 | CB | ASN | 42 | −0.144 | −19.703 | 18.507 | 1.00 | 0.06 |
| ATOM | 21 | CG | ASN | 42 | −0.362 | −20.112 | 17.072 | 1.00 | 0.57 |
| ATOM | 22 | OD1 | ASN | 42 | −1.415 | −19.951 | 16.465 | 1.00 | −0.57 |
| ATOM | 23 | ND2 | ASN | 42 | 0.695 | −20.754 | 16.486 | 1.00 | −0.80 |
| ATOM | 24 | N | VAL | 43 | 1.070 | −17.371 | 20.637 | 1.00 | −0.73 |
| ATOM | 25 | CA | VAL | 43 | 1.358 | −17.124 | 22.051 | 1.00 | 0.36 |
| ATOM | 26 | C | VAL | 43 | 0.696 | −15.775 | 22.438 | 1.00 | 0.57 |
| ATOM | 27 | O | VAL | 43 | 0.810 | −14.772 | 21.728 | 1.00 | −0.57 |
| ATOM | 28 | CB | VAL | 43 | 2.888 | −17.069 | 22.293 | 1.00 | 0.00 |
| ATOM | 29 | CG1 | VAL | 43 | 3.242 | −16.895 | 23.773 | 1.00 | 0.00 |
| ATOM | 30 | CG2 | VAL | 43 | 3.586 | −18.340 | 21.790 | 1.00 | 0.00 |
| ATOM | 31 | N | PRO | 44 | 0.031 | −15.695 | 23.647 | 1.00 | −0.66 |
| ATOM | 32 | CA | PRO | 44 | −0.680 | −14.469 | 24.048 | 1.00 | 0.36 |
| ATOM | 33 | C | PRO | 44 | 0.202 | −13.408 | 24.759 | 1.00 | 0.57 |
| ATOM | 34 | O | PRO | 44 | −0.291 | −12.537 | 25.475 | 1.00 | −0.57 |
| ATOM | 35 | CB | PRO | 44 | −1.770 | −14.981 | 24.999 | 1.00 | 0.00 |
| ATOM | 36 | CG | PRO | 44 | −1.131 | −16.210 | 25.637 | 1.00 | 0.00 |
| ATOM | 37 | CD | PRO | 44 | −0.321 | −16.814 | 24.502 | 1.00 | 0.30 |
| ATOM | 38 | N | GLU | 45 | 1.542 | −13.487 | 24.479 | 1.00 | −0.73 |
| ATOM | 39 | CA | GLU | 45 | 2.554 | −12.572 | 25.027 | 1.00 | 0.36 |
| ATOM | 40 | C | GLU | 45 | 2.867 | −11.393 | 24.065 | 1.00 | 0.57 |
| ATOM | 41 | O | GLU | 45 | 3.481 | −10.405 | 24.466 | 1.00 | −0.57 |
| ATOM | 42 | CB | GLU | 45 | 3.796 | −13.401 | 25.397 | 1.00 | 0.00 |
| ATOM | 43 | CG | GLU | 45 | 4.893 | −12.627 | 26.131 | 1.00 | −0.11 |
| ATOM | 44 | CD | GLU | 45 | 5.904 | −13.480 | 26.910 | 1.00 | 0.91 |
| ATOM | 45 | OE1 | GLU | 45 | 5.709 | −14.728 | 26.925 | 1.00 | −0.90 |
| ATOM | 46 | OE2 | GLU | 45 | 6.811 | −12.821 | 27.500 | 1.00 | −0.90 |
| ATOM | 47 | N | ASP | 46 | 2.437 | −11.550 | 22.758 | 1.00 | −0.73 |
| ATOM | 48 | CA | ASP | 46 | 2.879 | −10.670 | 21.649 | 1.00 | 0.36 |
| ATOM | 49 | C | ASP | 46 | 4.311 | −11.126 | 21.235 | 1.00 | 0.57 |
| ATOM | 50 | O | ASP | 46 | 4.827 | −12.151 | 21.690 | 1.00 | −0.57 |
| ATOM | 51 | CB | ASP | 46 | 2.726 | −9.185 | 21.971 | 1.00 | −0.11 |
| ATOM | 52 | CG | ASP | 46 | 2.615 | −8.268 | 20.761 | 1.00 | 0.91 |
| ATOM | 53 | OD1 | ASP | 46 | 2.845 | −8.794 | 19.636 | 1.00 | −0.90 |
| ATOM | 54 | OD2 | ASP | 46 | 2.293 | −7.071 | 21.018 | 1.00 | −0.90 |
| ATOM | 55 | N | GLU | 47 | 4.936 | −10.362 | 20.282 | 1.00 | −0.73 |
| ATOM | 56 | CA | GLU | 47 | 6.253 | −10.677 | 19.736 | 1.00 | 0.36 |
| ATOM | 57 | C | GLU | 47 | 6.940 | −9.327 | 19.392 | 1.00 | 0.57 |
| ATOM | 58 | O | GLU | 47 | 6.351 | −8.330 | 18.971 | 1.00 | −0.57 |
| ATOM | 59 | CB | GLU | 47 | 6.124 | −11.632 | 18.540 | 1.00 | 0.00 |
| ATOM | 60 | CG | GLU | 47 | 7.447 | −12.045 | 17.893 | 1.00 | −0.11 |
| ATOM | 61 | CD | GLU | 47 | 7.177 | −12.833 | 16.604 | 1.00 | 0.91 |
| ATOM | 62 | OE1 | GLU | 47 | 6.454 | −13.859 | 16.720 | 1.00 | −0.90 |
| ATOM | 63 | OE2 | GLU | 47 | 7.717 | −12.345 | 15.568 | 1.00 | −0.90 |
| ATOM | 64 | N | THR | 48 | 8.312 | −9.299 | 19.574 | 1.00 | −0.73 |
| ATOM | 65 | CA | THR | 48 | 9.076 | −8.068 | 19.335 | 1.00 | 0.36 |
| ATOM | 66 | C | THR | 48 | 9.395 | −7.933 | 17.832 | 1.00 | 0.57 |
| ATOM | 67 | O | THR | 48 | 10.451 | −8.322 | 17.332 | 1.00 | −0.57 |
| ATOM | 68 | CB | THR | 48 | 10.370 | −8.030 | 20.183 | 1.00 | 0.28 |
| ATOM | 69 | OG1 | THR | 48 | 10.026 | −7.847 | 21.567 | 1.00 | −0.68 |
| ATOM | 70 | OG2 | THR | 48 | 11.296 | −6.866 | 19.832 | 1.00 | 0.00 |
| ATOM | 71 | N | LEU | 49 | 8.393 | −7.340 | 17.090 | 1.00 | −0.73 |
| ATOM | 72 | CA | LEU | 49 | 8.541 | −7.129 | 15.654 | 1.00 | 0.36 |
| ATOM | 73 | C | LEU | 49 | 9.316 | −5.800 | 15.378 | 1.00 | 0.57 |
| ATOM | 74 | O | LEU | 49 | 9.561 | −4.938 | 16.224 | 1.00 | −0.57 |
| ATOM | 75 | CB | LEU | 49 | 7.198 | −7.145 | 14.904 | 1.00 | 0.00 |
| ATOM | 76 | CG | LEU | 49 | 6.592 | −8.539 | 14.626 | 1.00 | 0.00 |
| ATOM | 77 | CD1 | LEU | 49 | 7.556 | −9.480 | 13.901 | 1.00 | 0.00 |
| ATOM | 78 | CD2 | LEU | 49 | 6.064 | −9.203 | 15.888 | 1.00 | 0.00 |
| ATOM | 79 | N | ILE | 50 | 9.768 | −5.672 | 14.076 | 1.00 | −0.73 |
| ATOM | 80 | CA | ILE | 50 | 10.645 | −4.595 | 13.644 | 1.00 | 0.36 |
| ATOM | 81 | C | ILE | 50 | 9.788 | −3.571 | 12.859 | 1.00 | 0.57 |
| ATOM | 82 | O | ILE | 50 | 8.989 | −3.916 | 11.992 | 1.00 | −0.57 |
| ATOM | 83 | CB | ILE | 50 | 11.769 | −5.134 | 12.710 | 1.00 | 0.00 |
| ATOM | 84 | CG1 | ILE | 50 | 12.672 | −6.217 | 13.341 | 1.00 | 0.00 |
| ATOM | 85 | CG2 | ILE | 50 | 12.684 | −3.995 | 12.237 | 1.00 | 0.00 |
| ATOM | 86 | CD1 | ILE | 50 | 12.006 | −7.563 | 13.584 | 1.00 | 0.00 |
| ATOM | 87 | N | GLN | 51 | 10.070 | −2.232 | 13.120 | 1.00 | −0.73 |
| ATOM | 88 | CA | GLN | 51 | 9.280 | −1.203 | 12.420 | 1.00 | 0.36 |
| ATOM | 89 | C | GLN | 51 | 9.828 | −0.957 | 10.987 | 1.00 | 0.57 |
| ATOM | 90 | O | GLN | 51 | 9.124 | −0.551 | 10.064 | 1.00 | −0.57 |
| ATOM | 91 | CB | GLN | 51 | 9.182 | 0.109 | 13.222 | 1.00 | 0.00 |
| ATOM | 92 | CG | GLN | 51 | 10.509 | 0.828 | 13.457 | 1.00 | 0.06 |
| ATOM | 93 | CD | GLN | 51 | 10.479 | 2.075 | 14.321 | 1.00 | 0.57 |
| ATOM | 94 | OE1 | GLN | 51 | 11.513 | 2.687 | 14.580 | 1.00 | −0.57 |
| ATOM | 95 | NE2 | GLN | 51 | 9.293 | 2.493 | 14.828 | 1.00 | −0.80 |
| ATOM | 96 | N | GLN | 52 | 11.198 | −1.052 | 10.844 | 1.00 | −0.73 |
| ATOM | 97 | CA | GLN | 52 | 11.877 | −0.689 | 9.607 | 1.00 | 0.36 |
| ATOM | 98 | C | GLN | 52 | 11.779 | −1.788 | 8.521 | 1.00 | 0.57 |
| ATOM | 99 | O | GLN | 52 | 12.767 | −2.429 | 8.151 | 1.00 | −0.57 |
| ATOM | 100 | CB | GLN | 52 | 13.376 | −0.419 | 9.819 | 1.00 | 0.00 |
| ATOM | 101 | CG | GLN | 52 | 13.673 | 0.864 | 10.569 | 1.00 | 0.06 |
| ATOM | 102 | CD | GLN | 52 | 14.016 | 0.774 | 12.035 | 1.00 | 0.57 |
| ATOM | 103 | OE1 | GLN | 52 | 14.624 | 1.695 | 12.574 | 1.00 | −0.57 |
| ATOM | 104 | NE2 | GLN | 52 | 13.658 | −0.334 | 12.732 | 1.00 | −0.80 |
| ATOM | 105 | N | ILE | 53 | 10.546 | −1.932 | 7.943 | 1.00 | −0.73 |
| ATOM | 106 | CA | ILE | 53 | 10.362 | −2.770 | 6.757 | 1.00 | 0.36 |
| ATOM | 107 | C | ILE | 53 | 10.907 | −1.982 | 5.523 | 1.00 | 0.57 |
| ATOM | 108 | O | ILE | 53 | 10.936 | −0.748 | 5.480 | 1.00 | −0.57 |
| ATOM | 109 | CB | ILE | 53 | 8.894 | −3.234 | 6.641 | 1.00 | 0.00 |
| ATOM | 110 | CG1 | ILE | 53 | 8.692 | −4.461 | 5.736 | 1.00 | 0.00 |
| ATOM | 111 | CG2 | ILE | 53 | 7.951 | −2.112 | 6.185 | 1.00 | 0.00 |
| ATOM | 112 | CD1 | ILE | 53 | 9.474 | −5.693 | 6.165 | 1.00 | 0.00 |
| ATOM | 113 | N | LYS | 54 | 11.409 | −2.750 | 4.482 | 1.00 | −0.73 |
| ATOM | 114 | CA | LYS | 54 | 11.666 | −2.144 | 3.172 | 1.00 | 0.36 |
| ATOM | 115 | C | LYS | 54 | 10.412 | −2.358 | 2.293 | 1.00 | 0.57 |
| ATOM | 116 | O | LYS | 54 | 9.660 | −3.280 | 2.484 | 1.00 | −0.57 |
| ATOM | 117 | CB | LYS | 54 | 12.924 | −2.713 | 2.480 | 1.00 | 0.00 |
| ATOM | 118 | CG | LYS | 54 | 12.872 | −4.215 | 2.164 | 1.00 | 0.00 |
| ATOM | 119 | CD | LYS | 54 | 14.009 | −4.674 | 1.228 | 1.00 | 0.00 |
| ATOM | 120 | CE | LYS | 54 | 13.901 | −6.178 | 1.000 | 1.00 | 0.50 |
| ATOM | 121 | NZ | LYS | 54 | 14.859 | −6.667 | 0.002 | 1.00 | −0.85 |
| ATOM | 122 | N | ASP | 55 | 10.274 | −1.460 | 1.243 | 1.00 | −0.73 |
| ATOM | 123 | CA | ASP | 55 | 9.338 | −1.783 | 0.163 | 1.00 | 0.36 |
| ATOM | 124 | C | ASP | 55 | 10.145 | −2.474 | −0.961 | 1.00 | 0.57 |
| ATOM | 125 | O | ASP | 55 | 11.375 | −2.416 | −1.024 | 1.00 | −0.57 |
| ATOM | 126 | CB | ASP | 55 | 8.564 | −0.560 | −0.314 | 1.00 | −0.11 |
| ATOM | 127 | CG | ASP | 55 | 9.271 | 0.163 | −1.431 | 1.00 | 0.91 |
| ATOM | 128 | OD1 | ASP | 55 | 8.923 | −0.100 | −2.614 | 1.00 | −0.90 |
| ATOM | 129 | OD2 | ASP | 55 | 10.147 | 1.011 | −1.090 | 1.00 | −0.90 |
| ATOM | 130 | N | MET | 56 | 9.366 | −3.104 | −1.906 | 1.00 | −0.73 |
| ATOM | 131 | CA | MET | 56 | 9.882 | −3.302 | −3.251 | 1.00 | 0.36 |
| ATOM | 132 | C | MET | 56 | 8.663 | −3.400 | −4.193 | 1.00 | 0.57 |
| ATOM | 133 | O | MET | 56 | 7.659 | −4.057 | −3.920 | 1.00 | −0.57 |
| ATOM | 134 | CB | MET | 56 | 10.738 | −4.566 | −3.393 | 1.00 | 0.00 |
| ATOM | 135 | CG | MET | 56 | 11.715 | −4.427 | −4.563 | 1.00 | 0.23 |
| ATOM | 136 | SD | MET | 56 | 12.417 | −6.029 | −5.053 | 1.00 | −0.46 |
| ATOM | 137 | CE | MET | 56 | 11.094 | −6.599 | −6.148 | 1.00 | 0.23 |
| ATOM | 138 | N | VAL | 57 | 8.812 | −2.684 | −5.353 | 1.00 | −0.73 |
| ATOM | 139 | CA | VAL | 57 | 7.838 | −2.708 | −6.444 | 1.00 | 0.36 |
| ATOM | 140 | C | VAL | 57 | 8.487 | −3.633 | −7.492 | 1.00 | 0.57 |
| ATOM | 141 | O | VAL | 57 | 9.660 | −3.469 | −7.836 | 1.00 | −0.57 |
| ATOM | 142 | CB | VAL | 57 | 7.651 | −1.265 | −6.965 | 1.00 | 0.00 |
| ATOM | 143 | CG1 | VAL | 57 | 6.739 | −1.216 | −8.182 | 1.00 | 0.00 |
| ATOM | 144 | CG2 | VAL | 57 | 7.080 | −0.342 | −5.881 | 1.00 | 0.00 |
| ATOM | 145 | N | THR | 58 | 7.713 | −4.667 | −7.968 | 1.00 | −0.73 |
| ATOM | 146 | CA | THR | 58 | 8.225 | −5.554 | −9.023 | 1.00 | 0.36 |
| ATOM | 147 | C | THR | 58 | 8.028 | −4.921 | −10.415 | 1.00 | 0.57 |
| ATOM | 148 | O | THR | 58 | 8.764 | −5.183 | −11.364 | 1.00 | −0.57 |
| ATOM | 149 | CB | THR | 58 | 7.563 | −6.945 | −9.029 | 1.00 | 0.28 |
| ATOM | 150 | OG1 | THR | 58 | 6.135 | −6.841 | −9.071 | 1.00 | −0.68 |
| ATOM | 151 | CG2 | THR | 58 | 7.939 | −7.767 | −7.801 | 1.00 | 0.00 |
| ATOM | 152 | N | GLN | 59 | 6.893 | −4.162 | −10.544 | 1.00 | −0.73 |
| ATOM | 153 | CA | GLN | 59 | 6.518 | −3.457 | −11.763 | 1.00 | 0.36 |
| ATOM | 154 | C | GLN | 59 | 5.703 | −2.243 | −11.287 | 1.00 | 0.57 |
| ATOM | 155 | O | GLN | 59 | 4.911 | −2.330 | −10.349 | 1.00 | −0.57 |
| ATOM | 156 | CB | GLN | 59 | 5.681 | −4.345 | −12.687 | 1.00 | 0.00 |
| ATOM | 157 | CG | GLN | 59 | 5.366 | −3.662 | −14.015 | 1.00 | 0.06 |
| ATOM | 158 | CD | GLN | 59 | 4.595 | −4.587 | −14.925 | 1.00 | 0.57 |
| ATOM | 159 | OE1 | GLN | 59 | 5.104 | −5.557 | −15.479 | 1.00 | −0.57 |
| ATOM | 160 | NE2 | GLN | 59 | 3.283 | −4.274 | −15.088 | 1.00 | −0.80 |
| ATOM | 161 | N | ALA | 60 | 5.973 | −1.070 | −11.955 | 1.00 | −0.73 |
| ATOM | 162 | CA | ALA | 60 | 5.017 | 0.035 | −11.935 | 1.00 | 0.36 |
| ATOM | 163 | C | ALA | 60 | 4.446 | 0.092 | −13.360 | 1.00 | 0.57 |
| ATOM | 164 | O | ALA | 60 | 5.135 | −0.225 | −14.335 | 1.00 | −0.57 |
| ATOM | 165 | CB | ALA | 60 | 5.714 | 1.352 | −11.627 | 1.00 | 0.00 |
| ATOM | 166 | N | SER | 61 | 3.154 | 0.567 | −13.464 | 1.00 | −0.73 |
| ATOM | 167 | CA | SER | 61 | 2.521 | 0.441 | −14.776 | 1.00 | 0.36 |
| ATOM | 168 | C | SER | 61 | 3.247 | 1.374 | −15.775 | 1.00 | 0.57 |
| ATOM | 169 | O | SER | 61 | 3.753 | 2.457 | −15.467 | 1.00 | −0.57 |
| ATOM | 170 | CB | SER | 61 | 1.038 | 0.808 | −14.743 | 1.00 | 0.28 |
| ATOM | 171 | OG | SER | 61 | 0.850 | 2.168 | −14.331 | 1.00 | −0.68 |
| ATOM | 172 | N | LEU | 62 | 3.183 | 0.962 | −17.098 | 1.00 | −0.73 |
| ATOM | 173 | CA | LEU | 62 | 4.075 | 1.563 | −18.098 | 1.00 | 0.36 |
| ATOM | 174 | C | LEU | 62 | 3.845 | 3.063 | −18.425 | 1.00 | 0.57 |
| ATOM | 175 | O | LEU | 62 | 4.534 | 3.672 | −19.243 | 1.00 | −0.57 |
| ATOM | 176 | CB | LEU | 62 | 4.040 | 0.763 | −19.419 | 1.00 | 0.00 |
| ATOM | 177 | CG | LEU | 62 | 2.796 | 0.973 | −20.317 | 1.00 | 0.00 |
| ATOM | 178 | CD1 | LEU | 62 | 2.990 | 0.237 | −21.645 | 1.00 | 0.00 |
| ATOM | 179 | CD2 | LEU | 62 | 1.492 | 0.515 | −19.666 | 1.00 | 0.00 |
| ATOM | 180 | N | TYR | 63 | 2.782 | 3.642 | −17.781 | 1.00 | −0.73 |
| ATOM | 181 | CA | TYR | 63 | 2.408 | 5.033 | −17.959 | 1.00 | 0.36 |
| ATOM | 182 | C | TYR | 63 | 3.295 | 5.989 | −17.124 | 1.00 | 0.57 |
| ATOM | 183 | O | TYR | 63 | 3.304 | 7.205 | −17.344 | 1.00 | −0.57 |
| ATOM | 184 | CB | TYR | 63 | 0.933 | 5.252 | −17.582 | 1.00 | 0.14 |
| ATOM | 185 | CG | TYR | 63 | −0.033 | 4.385 | −18.366 | 1.00 | −0.14 |
| ATOM | 186 | CD1 | TYR | 63 | −0.288 | 4.638 | −19.722 | 1.00 | −0.15 |
| ATOM | 187 | CD2 | TYR | 63 | −0.673 | 3.300 | −17.748 | 1.00 | −0.15 |
| ATOM | 188 | CE1 | TYR | 63 | −1.172 | 3.828 | −20.441 | 1.00 | −0.15 |
| ATOM | 189 | CE2 | TYR | 63 | −1.560 | 2.494 | −18.464 | 1.00 | −0.15 |
| ATOM | 190 | CZ | TYR | 63 | −1.806 | 2.767 | −19.803 | 1.00 | 0.08 |
| ATOM | 191 | OH | TYR | 63 | −2.686 | 1.967 | −20.468 | 1.00 | −0.53 |
| ATOM | 192 | N | LEU | 64 | 3.965 | 5.426 | −16.056 | 1.00 | −0.73 |
| ATOM | 193 | CA | LEU | 64 | 4.896 | 6.189 | −15.244 | 1.00 | 0.36 |
| ATOM | 194 | C | LEU | 64 | 6.359 | 5.745 | −15.508 | 1.00 | 0.57 |
| ATOM | 195 | O | LEU | 64 | 6.685 | 4.588 | −15.754 | 1.00 | −0.57 |
| ATOM | 196 | CB | LEU | 64 | 4.615 | 6.051 | −13.734 | 1.00 | 0.00 |
| ATOM | 197 | CG | LEU | 64 | 3.283 | 6.676 | −13.266 | 1.00 | 0.00 |
| ATOM | 198 | CD1 | LEU | 64 | 2.088 | 5.754 | −13.503 | 1.00 | 0.00 |
| ATOM | 199 | CD2 | LEU | 64 | 3.354 | 7.002 | −11.771 | 1.00 | 0.00 |
| ATOM | 200 | N | PHE | 65 | 7.293 | 6.757 | −15.335 | 1.00 | −0.73 |
| ATOM | 201 | CA | PHE | 65 | 8.706 | 6.427 | −15.074 | 1.00 | 0.36 |
| ATOM | 202 | C | PHE | 65 | 8.769 | 6.241 | −13.544 | 1.00 | 0.57 |
| ATOM | 203 | O | PHE | 65 | 8.110 | 6.964 | −12.785 | 1.00 | −0.57 |
| ATOM | 204 | CB | PHE | 65 | 9.612 | 7.587 | −15.508 | 1.00 | 0.14 |
| ATOM | 205 | CG | PHE | 65 | 11.082 | 7.314 | −15.314 | 1.00 | −0.14 |
| ATOM | 206 | CD1 | PHE | 65 | 11.803 | 6.579 | −16.263 | 1.00 | −0.15 |
| ATOM | 207 | CD2 | PHE | 65 | 11.739 | 7.764 | −14.160 | 1.00 | −0.15 |
| ATOM | 208 | GE1 | PHE | 65 | 13.154 | 6.294 | −16.056 | 1.00 | −0.15 |
| ATOM | 209 | CE2 | PHE | 65 | 13.086 | 7.466 | −13.950 | 1.00 | −0.15 |
| ATOM | 210 | CZ | PHE | 65 | 13.793 | 6.732 | −14.898 | 1.00 | −0.15 |
| ATOM | 211 | N | GLU | 66 | 9.609 | 5.252 | −13.070 | 1.00 | −0.73 |
| ATOM | 212 | CA | GLU | 66 | 9.395 | 4.762 | −11.708 | 1.00 | 0.36 |
| ATOM | 213 | C | GLU | 66 | 9.608 | 5.874 | −10.654 | 1.00 | 0.57 |
| ATOM | 214 | O | GLU | 66 | 8.893 | 5.947 | −9.651 | 1.00 | −0.57 |
| ATOM | 215 | CB | GLU | 66 | 10.122 | 3.444 | −11.406 | 1.00 | 0.00 |
| ATOM | 216 | CG | GLU | 66 | 11.644 | 3.504 | −11.348 | 1.00 | −0.11 |
| ATOM | 217 | CD | GLU | 66 | 12.112 | 4.260 | −10.110 | 1.00 | 0.91 |
| ATOM | 218 | OE1 | GLU | 66 | 11.677 | 3.841 | −9.004 | 1.00 | −0.90 |
| ATOM | 219 | OE2 | GLU | 66 | 12.780 | 5.301 | −10.372 | 1.00 | −0.90 |
| ATOM | 220 | N | ALA | 67 | 10.592 | 6.811 | −10.923 | 1.00 | −0.73 |
| ATOM | 221 | CA | ALA | 67 | 10.971 | 7.758 | −9.873 | 1.00 | 0.36 |
| ATOM | 222 | C | ALA | 67 | 9.802 | 8.707 | −9.551 | 1.00 | 0.57 |
| ATOM | 223 | O | ALA | 67 | 9.616 | 9.168 | −8.423 | 1.00 | −0.57 |
| ATOM | 224 | CB | ALA | 67 | 12.163 | 8.606 | −10.297 | 1.00 | 0.00 |
| ATOM | 225 | N | THR | 68 | 9.016 | 9.078 | −10.637 | 1.00 | −0.73 |
| ATOM | 226 | CA | THR | 68 | 7.828 | 9.886 | −10.398 | 1.00 | 0.36 |
| ATOM | 227 | C | THR | 68 | 6.655 | 9.077 | −9.812 | 1.00 | 0.57 |
| ATOM | 228 | O | THR | 68 | 5.652 | 9.653 | −9.390 | 1.00 | −0.57 |
| ATOM | 229 | CB | THR | 68 | 7.347 | 10.722 | −11.605 | 1.00 | 0.28 |
| ATOM | 230 | OG1 | THR | 68 | 6.380 | 11.705 | −11.187 | 1.00 | −0.68 |
| ATOM | 231 | CG2 | THR | 68 | 6.745 | 9.921 | −12.750 | 1.00 | 0.00 |
| ATOM | 232 | N | GLY | 69 | 6.766 | 7.712 | −9.837 | 1.00 | −0.73 |
| ATOM | 233 | CA | GLY | 69 | 5.872 | 6.857 | −9.084 | 1.00 | 0.36 |
| ATOM | 234 | C | GLY | 69 | 6.308 | 6.799 | −7.615 | 1.00 | 0.57 |
| ATOM | 235 | O | GLY | 69 | 5.495 | 6.871 | −6.691 | 1.00 | −0.57 |
| ATOM | 236 | N | LYS | 70 | 7.660 | 6.644 | −7.394 | 1.00 | −0.73 |
| ATOM | 237 | CA | LYS | 70 | 8.221 | 6.548 | −6.047 | 1.00 | 0.36 |
| ATOM | 238 | C | LYS | 70 | 7.995 | 7.879 | −5.290 | 1.00 | 0.57 |
| ATOM | 239 | O | LYS | 70 | 7.894 | 7.929 | 4.063 | 1.00 | −0.57 |
| ATOM | 240 | CB | LYS | 70 | 9.716 | 6.217 | −6.052 | 1.00 | 0.00 |
| ATOM | 241 | CG | LYS | 70 | 10.019 | 4.734 | −6.296 | 1.00 | 0.00 |
| ATOM | 242 | CD | LYS | 70 | 9.740 | 3.843 | −5.080 | 1.00 | 0.00 |
| ATOM | 243 | CE | LYS | 70 | 10.084 | 2.389 | −5.381 | 1.00 | 0.50 |
| ATOM | 244 | NZ | LYS | 70 | 9.915 | 1.592 | −4.167 | 1.00 | −0.85 |
| ATOM | 245 | N | ARG | 71 | 7.936 | 8.997 | −6.104 | 1.00 | −0.73 |
| ATOM | 246 | CA | ARG | 71 | 7.758 | 10.342 | −5.559 | 1.00 | 0.36 |
| ATOM | 247 | C | ARG | 71 | 6.378 | 10.440 | −4.873 | 1.00 | 0.57 |
| ATOM | 248 | O | ARG | 71 | 6.172 | 11.198 | −3.926 | 1.00 | −0.57 |
| ATOM | 249 | CB | ARG | 71 | 7.808 | 11.368 | −6.704 | 1.00 | 0.00 |
| ATOM | 250 | CG | ARG | 71 | 7.520 | 12.804 | −6.269 | 1.00 | 0.00 |
| ATOM | 251 | CD | ARG | 71 | 7.417 | 13.744 | −7.464 | 1.00 | 0.33 |
| ATOM | 252 | NE | ARG | 71 | 6.928 | 15.071 | −7.057 | 1.00 | −0.84 |
| ATOM | 253 | CZ | ARG | 71 | 5.660 | 15.358 | −6.716 | 1.00 | 1.20 |
| ATOM | 254 | NH1 | ARG | 71 | 4.685 | 14.450 | −6.755 | 1.00 | −0.97 |
| ATOM | 255 | NH2 | ARG | 71 | 5.355 | 16.601 | −6.326 | 1.00 | −0.97 |
| ATOM | 256 | N | PHE | 72 | 5.351 | 9.777 | −5.516 | 1.00 | −0.73 |
| ATOM | 257 | CA | PHE | 72 | 4.043 | 9.687 | −4.873 | 1.00 | 0.36 |
| ATOM | 258 | C | PHE | 72 | 4.022 | 8.552 | −3.834 | 1.00 | 0.57 |
| ATOM | 259 | O | PHE | 72 | 3.291 | 8.613 | −2.845 | 1.00 | −0.57 |
| ATOM | 260 | CB | PHE | 72 | 2.908 | 9.469 | −5.878 | 1.00 | 0.14 |
| ATOM | 261 | CG | PHE | 72 | 2.618 | 10.701 | −6.709 | 1.00 | −0.14 |
| ATOM | 262 | CD1 | PHE | 72 | 2.978 | 10.749 | −8.058 | 1.00 | −0.15 |
| ATOM | 263 | CD2 | PHE | 72 | 1.960 | 11.803 | −6.149 | 1.00 | −0.15 |
| ATOM | 264 | CE1 | PHE | 72 | 2.695 | 11.875 | −8.833 | 1.00 | −0.15 |
| ATOM | 265 | CE2 | PHE | 72 | 1.648 | 12.918 | −6.930 | 1.00 | −0.15 |
| ATOM | 266 | CZ | PHE | 72 | 2.022 | 12.957 | −8.272 | 1.00 | −0.15 |
| ATOM | 267 | N | TYR | 73 | 4.793 | 7.438 | −4.116 | 1.00 | −0.73 |
| ATOM | 268 | CA | TYR | 73 | 4.615 | 6.214 | −3.329 | 1.00 | 0.36 |
| ATOM | 269 | C | TYR | 73 | 4.925 | 6.514 | −1.854 | 1.00 | 0.57 |
| ATOM | 270 | O | TYR | 73 | 4.280 | 6.029 | −0.924 | 1.00 | −0.57 |
| ATOM | 271 | CD | TYR | 73 | 5.542 | 5.086 | −3.816 | 1.00 | 0.14 |
| ATOM | 272 | CG | TYR | 73 | 5.276 | 3.747 | −3.169 | 1.00 | −0.14 |
| ATOM | 273 | CD1 | TYR | 73 | 5.790 | 3.463 | −1.893 | 1.00 | −0.15 |
| ATOM | 274 | CD2 | TYR | 73 | 4.467 | 2.797 | −3.805 | 1.00 | −0.15 |
| ATOM | 275 | CE1 | TYR | 73 | 5.443 | 2.289 | −1.231 | 1.00 | −0.15 |
| ATOM | 276 | CE2 | TYR | 73 | 4.144 | 1.607 | −3.154 | 1.00 | −0.15 |
| ATOM | 277 | CZ | TYR | 73 | 4.615 | 1.378 | −1.868 | 1.00 | 0.08 |
| ATOM | 278 | OH | TYR | 73 | 4.236 | 0.237 | −1.233 | 1.00 | −0.53 |
| ATOM | 279 | N | PHE | 74 | 6.037 | 7.301 | −1.597 | 1.00 | −0.73 |
| ATOM | 280 | CA | PHE | 74 | 6.487 | 7.388 | −0.207 | 1.00 | 0.36 |
| ATOM | 281 | C | PHE | 74 | 5.415 | 8.093 | 0.665 | 1.00 | 0.57 |
| ATOM | 282 | O | PHE | 74 | 5.366 | 7.954 | 1.886 | 1.00 | −0.57 |
| ATOM | 283 | CB | PHE | 74 | 7.881 | 8.008 | −0.032 | 1.00 | 0.14 |
| ATOM | 284 | CG | PHE | 74 | 7.955 | 9.516 | 0.016 | 1.00 | −0.14 |
| ATOM | 285 | CD1 | PHE | 74 | 8.322 | 10.171 | 1.203 | 1.00 | −0.15 |
| ATOM | 286 | CD2 | PHE | 74 | 7.675 | 10.285 | −1.116 | 1.00 | −0.15 |
| ATOM | 287 | CE1 | PHE | 74 | 8.404 | 11.564 | 1.252 | 1.00 | −0.15 |
| ATOM | 288 | CE2 | PHE | 74 | 7.742 | 11.678 | −1.062 | 1.0 | 0−0.15 |
| ATOM | 289 | CZ | PHE | 74 | 8.108 | 12.319 | 0.120 | 1.00 | −0.15 |
| ATOM | 290 | N | LYS | 75 | 4.579 | 8.951 | −0.019 | 1.00 | −0.73 |
| ATOM | 291 | CA | LYS | 75 | 3.446 | 9.633 | 0.585 | 1.00 | 0.36 |
| ATOM | 292 | C | LYS | 75 | 2.083 | 9.047 | 0.153 | 1.00 | 0.57 |
| ATOM | 293 | O | LYS | 75 | 1.054 | 9.725 | 0.143 | 1.00 | −0.57 |
| ATOM | 294 | CB | LYS | 75 | 3.527 | 11.146 | 0.307 | 1.00 | 0.00 |
| ATOM | 295 | CG | LYS | 75 | 4.546 | 11.853 | 1.210 | 1.00 | 0.00 |
| ATOM | 296 | CD | LYS | 75 | 4.016 | 12.072 | 2.630 | 1.00 | 0.00 |
| ATOM | 297 | CE | LYS | 75 | 5.118 | 12.545 | 3.568 | 1.00 | 0.50 |
| ATOM | 298 | NZ | LYS | 75 | 4.568 | 12.679 | 4.933 | 1.00 | −0.85 |
| ATOM | 299 | N | ASN | 76 | 2.050 | 7.681 | −0.037 | 1.00 | −0.73 |
| ATOM | 300 | CA | ASN | 76 | 0.815 | 6.935 | 0.214 | 1.00 | 0.36 |
| ATOM | 301 | C | ASN | 76 | 0.854 | 6.435 | 1.675 | 1.00 | 0.57 |
| ATOM | 302 | O | ASN | 76 | −0.089 | 6.608 | 2.444 | 1.00 | −0.57 |
| ATOM | 303 | CB | ASN | 76 | 0.500 | 5.881 | −0.841 | 1.00 | 0.06 |
| ATOM | 304 | CG | ASN | 76 | 1.537 | 4.805 | −0.996 | 1.00 | 0.57 |
| ATOM | 305 | OD1 | ASN | 76 | 2.127 | 4.286 | −0.045 | 1.00 | −0.57 |
| ATOM | 306 | ND2 | ASN | 76 | 1.796 | 4.396 | −2.261 | 1.00 | −0.80 |
| ATOM | 307 | N | VAL | 77 | 2.019 | 5.789 | 2.059 | 1.00 | −0.73 |
| ATOM | 308 | CA | VAL | 77 | 2.080 | 5.190 | 3.390 | 1.00 | 0.36 |
| ATOM | 309 | C | VAL | 77 | 2.289 | 6.298 | 4.451 | 1.00 | 0.57 |
| ATOM | 310 | O | VAL | 77 | 1.748 | 6.276 | 5.559 | 1.00 | −0.57 |
| ATOM | 311 | CB | VAL | 77 | 3.177 | 4.108 | 3.518 | 1.00 | 0.00 |
| ATOM | 312 | CG1 | VAL | 77 | 2.751 | 2.788 | 2.873 | 1.00 | 0.00 |
| ATOM | 313 | CG2 | VAL | 77 | 4.554 | 4.519 | 2.989 | 1.00 | 0.00 |
| ATOM | 314 | N | ALA | 78 | 3.203 | 7.286 | 4.132 | 1.00 | −0.73 |
| ATOM | 315 | CA | ALA | 78 | 3.752 | 8.169 | 5.155 | 1.00 | 0.36 |
| ATOM | 316 | C | ALA | 78 | 2.846 | 9.385 | 5.470 | 1.00 | 0.57 |
| ATOM | 317 | O | ALA | 78 | 3.291 | 10.533 | 5.579 | 1.00 | −0.57 |
| ATOM | 318 | CB | ALA | 78 | 5.158 | 8.635 | 4.792 | 1.00 | 0.00 |
| ATOM | 319 | N | ILE | 79 | 1.539 | 9.066 | 5.735 | 1.00 | −0.73 |
| ATOM | 320 | CA | ILE | 79 | 0.480 | 10.036 | 6.010 | 1.00 | 0.36 |
| ATOM | 321 | C | ILE | 79 | −0.725 | 9.292 | 6.643 | 1.00 | 0.57 |
| ATOM | 322 | O | ILE | 79 | −1.894 | 9.497 | 6.326 | 1.00 | −0.57 |
| ATOM | 323 | CB | ILE | 79 | 0.143 | 10.898 | 4.761 | 1.00 | 0.00 |
| ATOM | 324 | CG1 | ILE | 79 | −0.811 | 12.058 | 5.120 | 1.00 | 0.00 |
| ATOM | 325 | CG2 | ILE | 79 | −0.367 | 10.061 | 3.582 | 1.00 | 0.00 |
| ATOM | 326 | CD1 | ILE | 79 | −0.938 | 13.108 | 4.025 | 1.00 | 0.00 |
| ATOM | 327 | N | LEU | 80 | −0.378 | 8.454 | 7.691 | 1.00 | −0.73 |
| ATOM | 328 | CA | LEU | 80 | −1.387 | 7.590 | 8.304 | 1.00 | 0.36 |
| ATOM | 329 | C | LEU | 80 | −1.177 | 7.542 | 9.834 | 1.00 | 0.57 |
| ATOM | 330 | O | LEU | 80 | −1.956 | 8.099 | 10.609 | 1.00 | −0.57 |
| ATOM | 331 | CB | LEU | 80 | −1.385 | 6.210 | 7.618 | 1.00 | 0.00 |
| ATOM | 332 | CG | LEU | 80 | −2.765 | 5.526 | 7.568 | 1.00 | 0.00 |
| ATOM | 333 | CD1 | LEU | 80 | −2.666 | 4.222 | 6.769 | 1.00 | 0.00 |
| ATOM | 334 | CD2 | LEU | 80 | −3.342 | 5.238 | 8.951 | 1.00 | 0.00 |
| ATOM | 335 | N | ILE | 81 | −0.074 | 6.855 | 10.285 | 1.00 | −0.73 |
| ATOM | 336 | CA | ILE | 81 | 0.124 | 6.532 | 11.715 | 1.00 | 0.36 |
| ATOM | 337 | C | ILE | 81 | 1.622 | 6.135 | 11.854 | 1.00 | 0.57 |
| ATOM | 338 | O | ILE | 81 | 2.238 | 5.734 | 10.856 | 1.00 | −0.57 |
| ATOM | 339 | CB | ILE | 81 | −0.854 | 5.396 | 12.131 | 1.00 | 0.00 |
| ATOM | 340 | CG1 | ILE | 81 | −1.044 | 5.206 | 13.647 | 1.00 | 0.00 |
| ATOM | 341 | CG2 | ILE | 81 | −0.468 | 4.050 | 11.500 | 1.00 | 0.00 |
| ATOM | 342 | CD1 | ILE | 81 | −1.682 | 6.396 | 14.350 | 1.00 | 0.00 |
| ATOM | 343 | N | PRO | 82 | 2.223 | 6.154 | 13.099 | 1.00 | −0.66 |
| ATOM | 344 | CA | PRO | 82 | 3.639 | 5.759 | 13.267 | 1.00 | 0.36 |
| ATOM | 345 | C | PRO | 82 | 3.988 | 4.251 | 13.139 | 1.00 | 0.57 |
| ATOM | 346 | O | PRO | 82 | 4.641 | 3.636 | 13.983 | 1.00 | −0.57 |
| ATOM | 347 | CB | PRO | 82 | 4.003 | 6.272 | 14.665 | 1.00 | 0.00 |
| ATOM | 348 | CG | PRO | 82 | 3.145 | 7.514 | 14.806 | 1.00 | 0.00 |
| ATOM | 349 | CD | PRO | 82 | 1.838 | 7.084 | 14.159 | 1.00 | 0.30 |
| ATOM | 350 | N | GLU | 83 | 3.644 | 3.677 | 11.930 | 1.00 | −0.73 |
| ATOM | 351 | CA | GLU | 83 | 4.482 | 2.596 | 11.391 | 1.00 | 0.36 |
| ATOM | 352 | C | GLU | 83 | 5.715 | 3.293 | 10.738 | 1.00 | 0.57 |
| ATOM | 353 | O | GLU | 83 | 5.890 | 4.513 | 10.820 | 1.00 | −0.57 |
| ATOM | 354 | CB | GLU | 83 | 3.699 | 1.772 | 10.316 | 1.00 | 0.00 |
| ATOM | 355 | CG | GLU | 83 | 2.492 | 1.036 | 10.950 | 1.00 | −0.11 |
| ATOM | 356 | CD | GLU | 83 | 1.629 | 0.466 | 9.830 | 1.00 | 0.91 |
| ATOM | 357 | OE1 | GLU | 83 | 2.065 | 0.533 | 8.654 | 1.00 | −0.90 |
| ATOM | 358 | OE2 | GLU | 83 | 0.503 | 0.008 | 10.218 | 1.00 | −0.90 |
| ATOM | 359 | N | THR | 84 | 6.649 | 2.483 | 10.120 | 1.00 | −0.73 |
| ATOM | 360 | CA | THR | 84 | 7.729 | 3.099 | 9.334 | 1.00 | 0.36 |
| ATOM | 361 | C | THR | 84 | 7.977 | 2.225 | 8.090 | 1.00 | 0.57 |
| ATOM | 362 | O | THR | 84 | 7.642 | 1.041 | 8.065 | 1.00 | −0.57 |
| ATOM | 363 | CB | THR | 84 | 9.048 | 3.280 | 10.120 | 1.00 | 0.28 |
| ATOM | 364 | OG1 | THR | 84 | 9.724 | 2.047 | 10.390 | 1.00 | −0.68 |
| ATOM | 365 | CG2 | THR | 84 | 8.874 | 3.987 | 11.458 | 1.00 | 0.00 |
| ATOM | 366 | N | TRP | 85 | 8.635 | 2.840 | 7.040 | 1.00 | −0.73 |
| ATOM | 367 | CA | TRP | 85 | 8.593 | 2.220 | 5.708 | 1.00 | 0.36 |
| ATOM | 368 | C | TRP | 85 | 9.879 | 2.686 | 5.002 | 1.00 | 0.57 |
| ATOM | 369 | O | TRP | 85 | 9.980 | 3.751 | 4.386 | 1.00 | −0.57 |
| ATOM | 370 | CB | TRP | 85 | 7.354 | 2.643 | 4.887 | 1.00 | 0.18 |
| ATOM | 371 | CG | TRP | 85 | 6.080 | 2.714 | 5.675 | 1.00 | −0.18 |
| ATOM | 372 | CD1 | TRP | 85 | 5.159 | 1.706 | 5.884 | 1.00 | −0.30 |
| ATOM | 373 | CD2 | TRP | 85 | 5.617 | 3.856 | 6.406 | 1.00 | 0.00 |
| ATOM | 374 | NE1 | TRP | 85 | 4.177 | 2.173 | 6.725 | 1.00 | 0.03 |
| ATOM | 375 | CE2 | TRP | 85 | 4.426 | 3.496 | 7.033 | 1.00 | −0.15 |
| ATOM | 376 | CE3 | TRP | 85 | 6.125 | 5.157 | 6.613 | 1.00 | −0.15 |
| ATOM | 377 | CZ2 | TRP | 85 | 3.696 | 4.392 | 7.821 | 1.00 | −0.15 |
| ATOM | 378 | CZ3 | TRP | 85 | 5.455 | 6.027 | 7.476 | 1.00 | −0.15 |
| ATOM | 379 | CH2 | TRP | 85 | 4.249 | 5.651 | 8.059 | 1.00 | −0.15 |
| ATOM | 380 | N | LYS | 86 | 10.987 | 1.900 | 5.213 | 1.00 | −0.73 |
| ATOM | 381 | CA | LYS | 86 | 12.335 | 2.318 | 4.855 | 1.00 | 0.36 |
| ATOM | 382 | C | LYS | 86 | 12.607 | 2.096 | 3.333 | 1.00 | 0.57 |
| ATOM | 383 | O | LYS | 86 | 13.477 | 1.346 | 2.927 | 1.00 | −0.57 |
| ATOM | 384 | CB | LYS | 86 | 13.367 | 1.571 | 5.735 | 1.00 | 0.00 |
| ATOM | 385 | CG | LYS | 86 | 14.575 | 2.457 | 6.079 | 1.00 | 0.00 |
| ATOM | 386 | CD | LYS | 86 | 15.681 | 1.684 | 6.812 | 1.00 | 0.00 |
| ATOM | 387 | CE | LYS | 86 | 16.720 | 2.598 | 7.466 | 1.00 | 0.50 |
| ATOM | 388 | NZ | LYS | 86 | 16.238 | 3.108 | 8.770 | 1.00 | −0.85 |
| ATOM | 389 | N | THR | 87 | 11.881 | 2.930 | 2.496 | 1.00 | −0.73 |
| ATOM | 390 | CA | THR | 87 | 11.636 | 2.635 | 1.065 | 1.00 | 0.36 |
| ATOM | 391 | C | THR | 87 | 12.915 | 2.326 | 0.240 | 1.00 | 0.57 |
| ATOM | 392 | O | THR | 87 | 14.009 | 2.846 | 0.478 | 1.00 | −0.57 |
| ATOM | 393 | CB | THR | 87 | 10.860 | 3.804 | 0.391 | 1.00 | 0.28 |
| ATOM | 394 | OG1 | THR | 87 | 10.485 | 3.535 | −0.962 | 1.00 | −0.68 |
| ATOM | 395 | CG2 | THR | 87 | 11.632 | 5.123 | 0.399 | 1.00 | 0.00 |
| ATOM | 396 | N | LYS | 88 | 12.764 | 1.392 | −0.759 | 1.00 | −0.73 |
| ATOM | 397 | CA | LYS | 88 | 13.855 | 0.883 | −1.594 | 1.00 | 0.36 |
| ATOM | 398 | C | LYS | 88 | 13.399 | 0.695 | −3.058 | 1.00 | 0.57 |
| ATOM | 399 | O | LYS | 88 | 12.230 | 0.822 | −3.411 | 1.00 | −0.57 |
| ATOM | 400 | CB | LYS | 88 | 14.345 | −0.458 | −1.011 | 1.00 | 0.00 |
| ATOM | 401 | CG | LYS | 88 | 15.869 | −0.608 | −0.880 | 1.00 | 0.00 |
| ATOM | 402 | CD | LYS | 88 | 16.582 | 0.454 | −0.032 | 1.00 | 0.00 |
| ATOM | 403 | CE | LYS | 88 | 16.100 | 0.504 | 1.411 | 1.00 | 0.50 |
| ATOM | 404 | NZ | LYS | 88 | 15.856 | 1.895 | 1.813 | 1.00 | −.0.85 |
| ATOM | 405 | N | ALA | 89 | 14.406 | 0.337 | −3.931 | 1.00 | −0.73 |
| ATOM | 406 | CA | ALA | 89 | 14.168 | 0.209 | −5.376 | 1.00 | 0.36 |
| ATOM | 407 | C | ALA | 89 | 15.119 | −0.847 | −5.973 | 1.00 | 0.57 |
| ATOM | 408 | O | ALA | 89 | 15.558 | −0.790 | −7.116 | 1.00 | −0.57 |
| ATOM | 409 | CB | ALA | 89 | 14.346 | 1.557 | −6.066 | 1.00 | 0.00 |
| ATOM | 410 | N | ASP | 90 | 15.367 | −1.929 | −5.149 | 1.00 | −0.73 |
| ATOM | 411 | CA | ASP | 90 | 16.340 | −2.952 | −5535 | 1.00 | 0.36 |
| ATOM | 412 | C | ASP | 90 | 16.135 | −4.205 | −4.653 | 1.00 | 0.57 |
| ATOM | 413 | O | ASP | 90 | 15.834 | 4.124 | −3.452 | 1.00 | −0.57 |
| ATOM | 414 | CB | ASP | 90 | 17.753 | −2.421 | −5.338 | 1.00 | −0.11 |
| ATOM | 415 | CG | ASP | 90 | 18.838 | 3.272 | −5.933 | 1.00 | 0.91 |
| ATOM | 416 | OD1 | ASP | 90 | 18.559 | −4.385 | −6.468 | 1.00 | −0.90 |
| ATOM | 417 | OD2 | ASP | 90 | 20.024 | −2.835 | −5.817 | 1.00 | −0.90 |
| ATOM | 418 | N | TYR | 91 | 16.346 | −5.389 | −5.319 | 1.00 | −0.73 |
| ATOM | 419 | CA | TYR | 91 | 16.595 | −6.671 | −4.675 | 1.00 | 0.36 |
| ATOM | 420 | C | TYR | 91 | 15.548 | −7.154 | −3.642 | 1.00 | 0.57 |
| ATOM | 421 | O | TYR | 91 | 15.416 | −6.708 | −2.501 | 1.00 | −0.57 |
| ATOM | 422 | CB | TYR | 91 | 18.002 | −6.763 | 4.058 | 1.00 | 0.14 |
| ATOM | 423 | CG | TYR | 91 | 18.845 | −7.870 | −4.671 | 1.00 | −0.14 |
| ATOM | 424 | CD1 | TYR | 91 | 19.204 | −7.847 | −6.027 | 1.00 | −0.15 |
| ATOM | 425 | CD2 | TYR | 91 | 19.310 | −8.924 | −3.877 | 1.00 | −0.15 |
| ATOM | 426 | CE1 | TYR | 91 | 20.005 | −8.857 | −6.573 | 1.00 | −0.15 |
| ATOM | 427 | CE2 | TYR | 91 | 20.117 | −9.927 | 4.414 | 1.00 | −0.15 |
| ATOM | 428 | CZ | TYR | 91 | 20.456 | −9.890 | −5.758 | 1.00 | 0.08 |
| ATOM | 429 | OH | TYR | 91 | 21.241 | −10.895 | −6.237 | 1.00 | −0.53 |
| ATOM | 430 | N | VAL | 92 | 14.798 | −8.235 | 4.094 | 1.00 | −0.73 |
| ATOM | 431 | CA | VAL | 92 | 13.786 | −8.821 | −3.206 | 1.00 | 0.36 |
| ATOM | 432 | C | VAL | 92 | 14.527 | −9.419 | −1.983 | 1.00 | 0.57 |
| ATOM | 433 | O | VAL | 92 | 14.196 | −9.189 | −0.820 | 1.00 | −0.57 |
| ATOM | 434 | CD | VAL | 92 | 12.912 | −9.849 | −3.966 | 1.00 | 0.00 |
| ATOM | 435 | CG1 | VAL | 92 | 13.689 | −10.944 | 4.703 | 1.00 | 0.00 |
| ATOM | 436 | CG2 | VAL | 92 | 11.866 | −10.486 | −3.052 | 1.00 | 0.00 |
| ATOM | 437 | N | ARG | 93 | 15.588 | −10.233 | −2.300 | 1.00 | −0.73 |
| ATOM | 438 | CA | ARG | 93 | 16.404 | −10.870 | −1.269 | 1.00 | 0.36 |
| ATOM | 439 | C | ARG | 93 | 17.412 | −9.847 | −0.675 | 1.00 | 0.57 |
| ATOM | 440 | O | ARG | 93 | 17.419 | −8.658 | −1.015 | 1.00 | −0.57 |
| ATOM | 441 | CD | ARG | 93 | 17.079 | −12.099 | −1.908 | 1.00 | 0.00 |
| ATOM | 442 | CG | ARG | 93 | 16.131 | −13.306 | −1.891 | 1.00 | 0.00 |
| ATOM | 443 | CD | ARG | 93 | 16.696 | −14.534 | −2.595 | 1.00 | 0.33 |
| ATOM | 444 | NE | ARG | 93 | 17.794 | −15.153 | −1.835 | 1.00 | −0.84 |
| ATOM | 445 | CZ | ARG | 93 | 19.108 | −15.004 | −2.057 | 1.00 | 1.20 |
| ATOM | 446 | NH1 | ARG | 93 | 19.588 | −14.193 | −3.002 | 1.00 | −0.97 |
| ATOM | 447 | NH2 | ARG | 93 | 19.977 | −15.678 | −1.296 | 1.00 | −0.97 |
| ATOM | 448 | N | PRO | 94 | 18.313 | −10.296 | 0.264 | 1.00 | −0.66 |
| ATOM | 449 | CA | PRO | 94 | 19.409 | −9.432 | 0.722 | 1.00 | 0.36 |
| ATOM | 450 | C | PRO | 94 | 20.461 | −9.291 | −0.397 | 1.00 | 0.57 |
| ATOM | 451 | O | PRO | 94 | 20.852 | −10.264 | −1.042 | 1.00 | −0.57 |
| ATOM | 452 | CB | PRO | 94 | 20.014 | −10.180 | 1.912 | 1.00 | 0.00 |
| ATOM | 453 | CG | PRO | 94 | 18.882 | −11.082 | 2.389 | 1.00 | 0.00 |
| ATOM | 454 | CD | PRO | 94 | 18.193 | −1L480 | 1.098 | 1.00 | 0.30 |
| ATOM | 455 | N | LYS | 95 | 20.896 | −8.002 | −0.625 | 1.00 | −0.73 |
| ATOM | 456 | CA | LYS | 95 | 21.964 | −7.687 | −1593 | 1.00 | 0.36 |
| ATOM | 457 | C | LYS | 95 | 23.301 | −7.411 | −0.875 | 1.00 | 0.57 |
| ATOM | 458 | O | LYS | 95 | 24.351 | −7.293 | −1.497 | 1.00 | −0.57 |
| ATOM | 459 | CB | LYS | 95 | 21.578 | −6.446 | −2.420 | 1.00 | 0.00 |
| ATOM | 460 | CG | LYS | 95 | 22.251 | −6.426 | −3.804 | 1.00 | 0.00 |
| ATOM | 461 | CD | LYS | 95 | 21.687 | −5.307 | −4.681 | 1.00 | 0.00 |
| ATOM | 462 | CE | LYS | 95 | 22.148 | −5.404 | −6.131 | 1.00 | 0.50 |
| ATOM | 463 | NZ | LYS | 95 | 21.304 | −4.541 | −6.964 | 1.00 | −0.85 |
| ATOM | 464 | N | LEU | 96 | 23.176 | −7.164 | 0.474 | 1.00 | −0.73 |
| ATOM | 465 | CA | LEU | 96 | 24.326 | −6.832 | 1.323 | 1.00 | 0.36 |
| ATOM | 466 | C | LEU | 96 | 24.282 | −7.821 | 2.514 | 1.00 | 0.57 |
| ATOM | 467 | O | LEU | 96 | 23.303 | −8.546 | 2.724 | 1.00 | −0.57 |
| ATOM | 468 | CE | LEU | 96 | 24.215 | −5.384 | 1.824 | 1.00 | 0.00 |
| ATOM | 469 | CG | LEU | 96 | 24.185 | −4.316 | 0.712 | 1.00 | 0.00 |
| ATOM | 470 | CD1 | LEU | 96 | 23.885 | −2.943 | 1.319 | 1.00 | 0.00 |
| ATOM | 471 | CD2 | LEU | 96 | 25.498 | −4.255 | −0.069 | 1.00 | 0.00 |
| ATOM | 472 | N | GLU | 97 | 25.385 | −7.778 | 3.321 | 1.00 | −0.73 |
| ATOM | 473 | CA | GLU | 97 | 25.617 | −8.659 | 4.460 | 1.00 | 0.36 |
| ATOM | 474 | C | GLU | 97 | 24.578 | −8.460 | 5.586 | 1.00 | 0.57 |
| ATOM | 475 | O | GLU | 97 | 24.173 | −9.390 | 6.288 | 1.00 | −0.57 |
| ATOM | 476 | CD | GLU | 97 | 27.054 | −8.501 | 5.009 | 1.00 | 0.00 |
| ATOM | 477 | CG | GLU | 97 | 27.402 | −7.153 | 5.664 | 1.00 | −0.11 |
| ATOM | 478 | CD | GLU | 97 | 26.955 | −5.973 | 4.809 | 1.00 | 0.91 |
| ATOM | 479 | OE1 | GLU | 97 | 27.353 | −5.986 | 3.612 | 1.00 | −0.90 |
| ATOM | 480 | OE2 | GLU | 97 | 26.066 | −5.239 | 5.334 | 1.00 | −0.90 |
| ATOM | 481 | N | THR | 98 | 24.180 | −7.171 | 5.828 | 1.00 | −0.73 |
| ATOM | 482 | CA | THR | 98 | 23.380 | −6.765 | 6.977 | 1.00 | 0.36 |
| ATOM | 483 | C | THR | 98 | 21.888 | −7.046 | 6.680 | 1.00 | 0.57 |
| ATOM | 484 | O | THR | 98 | 20.989 | −6.210 | 6.793 | 1.00 | −0.57 |
| ATOM | 485 | CB | THR | 98 | 23.596 | −5.286 | 7.386 | 1.00 | 0.28 |
| ATOM | 486 | OG1 | THR | 98 | 23.758 | 4.431 | 6.254 | 1.00 | −0.68 |
| ATOM | 487 | CG2 | THR | 98 | 24.776 | −5.114 | 8.338 | 1.00 | 0.00 |
| ATOM | 488 | N | TYR | 99 | 21.576 | −8.373 | 6.478 | 1.00 | −0.73 |
| ATOM | 489 | CA | TYR | 99 | 20.382 | −8.803 | 5.759 | 1.00 | 0.36 |
| ATOM | 490 | C | TYR | 99 | 19.027 | −8.331 | 6.357 | 1.00 | 0.57 |
| ATOM | 491 | O | TYR | 99 | 18.820 | −8.168 | 7.560 | 1.00 | −0.57 |
| ATOM | 492 | CB | TYR | 99 | 20.340 | −10.344 | 5.625 | 1.00 | 0.14 |
| ATOM | 493 | CG | TYR | 99 | 20.412 | −11.080 | 6.947 | 1.00 | −0.14 |
| ATOM | 494 | CD1 | TYR | 99 | 21.644 | −11.545 | 7.432 | 1.00 | −0.15 |
| ATOM | 495 | CD2 | TYR | 99 | 19.266 | −11.251 | 7.737 | 1.00 | −0.15 |
| ATOM | 496 | CE1 | TYR | 99 | 21.736 | −12.129 | 8.697 | 1.00 | −0.15 |
| ATOM | 497 | CE2 | TYR | 99 | 19.360 | −11.823 | 9.005 | 1.00 | −0.15 |
| ATOM | 498 | CZ | TYR | 99 | 20.594 | −12.250 | 9.479 | 1.00 | 0.08 |
| ATOM | 499 | OH | TYR | 99 | 20.638 | −12.778 | 10.733 | 1.00 | −0.53 |
| ATOM | 500 | N | LYS | 100 | 18.021 | −8.145 | 5.425 | 1.00 | −0.73 |
| ATOM | 501 | CA | LYS | 100 | 16.600 | −8.070 | 5.792 | 1.00 | 0.36 |
| ATOM | 502 | C | LYS | 100 | 15.821 | −8.710 | 4.614 | 1.00 | 0.57 |
| ATOM | 503 | O | LYS | 100 | 16.280 | −8.708 | 3.468 | 1.00 | −0.57 |
| ATOM | 504 | CB | LYS | 100 | 16.096 | −6.635 | 6.032 | 1.00 | 0.00 |
| ATOM | 505 | CG | LYS | 100 | 16.607 | −5.989 | 7.329 | 1.00 | 0.00 |
| ATOM | 506 | CD | LYS | 100 | 17.871 | −5.142 | 7.134 | 1.00 | 0.00 |
| ATOM | 507 | CE | LYS | 100 | 18.626 | −4.893 | 8.440 | 1.00 | 0.50 |
| ATOM | 508 | NZ | LYS | 100 | 19.383 | −6.085 | 8.834 | 1.00 | −0.85 |
| ATOM | 509 | N | ASN | 101 | 14.615 | −9.271 | 4.953 | 1.00 | −0.73 |
| ATOM | 510 | CA | ASN | 101 | 13.745 | −9.928 | 3.963 | 1.00 | 0.36 |
| ATOM | 511 | C | ASN | 101 | 12.840 | −8.865 | 3.279 | 1.00 | 0.57 |
| ATOM | 512 | O | ASN | 101 | 12.745 | −7.706 | 3.682 | 1.00 | −0.57 |
| ATOM | 513 | CB | ASN | 101 | 12.932 | −11.019 | 4.661 | 1.00 | 0.06 |
| ATOM | 514 | CG | ASN | 101 | 12.176 | −11.883 | 3.685 | 1.00 | 0.57 |
| ATOM | 515 | OD1 | ASN | 101 | 12.490 | −11.983 | 2.499 | 1.00 | −0.57 |
| ATOM | 516 | ND2 | ASN | 101 | 11.140 | −12.587 | 4.208 | 1.00 | −0.80 |
| ATOM | 517 | N | ALA | 102 | 12.212 | −9.287 | 2.124 | 1.00 | −0.73 |
| ATOM | 518 | CA | ALA | 102 | 11.059 | −8.589 | 1.555 | 1.00 | 0.36 |
| ATOM | 519 | C | ALA | 102 | 9.797 | −9.337 | 2.007 | 1.00 | 0.57 |
| ATOM | 520 | O | ALA | 102 | 9.292 | −10.260 | 1.365 | 1.00 | −0.57 |
| ATOM | 521 | CB | ALA | 102 | 11.112 | −8.619 | 0.036 | 1.00 | 0.00 |
| ATOM | 522 | N | ASP | 103 | 9.320 | −8.940 | 3.235 | 1.00 | −0.73 |
| ATOM | 523 | CA | ASP | 103 | 8.204 | −9.683 | 3.836 | 1.00 | 0.36 |
| ATOM | 524 | C | ASP | 103 | 6.918 | −9.203 | 3.123 | 1.00 | 0.57 |
| ATOM | 525 | O | ASP | 103 | 5.948 | −9.946 | 2.944 | 1.00 | −0.57 |
| ATOM | 526 | CB | ASP | 103 | 8.113 | −9.478 | 5.338 | 1.00 | −0.11 |
| ATOM | 527 | CG | ASP | 103 | 9.441 | −10.045 | 5.789 | 1.00 | 0.91 |
| ATOM | 528 | OD1 | ASP | 103 | 10.384 | −9.205 | 5.864 | 1.00 | −0.90 |
| ATOM | 529 | OD2 | ASP | 103 | 9.513 | −11.305 | 5.873 | 1.00 | −0.90 |
| ATOM | 530 | N | VAL | 104 | 6.951 | −7.865 | 2.786 | 1.00 | −0.73 |
| ATOM | 531 | CA | VAL | 104 | 5.938 | −7.177 | 1.995 | 1.00 | 0.36 |
| ATOM | 532 | C | VAL | 104 | 6.495 | −6.965 | 0.560 | 1.00 | 0.57 |
| ATOM | 533 | O | VAL | 104 | 7.691 | −6.755 | 0.342 | 1.00 | −0.57 |
| ATOM | 534 | CB | VAL | 104 | 5.508 | −5.847 | 2.653 | 1.00 | 0.00 |
| ATOM | 535 | CG1 | VAL | 104 | 5.127 | −6.067 | 4.123 | 1.00 | 0.00 |
| ATOM | 536 | CG2 | VAL | 104 | 6.564 | 4.742 | 2.563 | 1.00 | 0.00 |
| ATOM | 537 | N | LEU | 105 | 5.556 | −7.029 | −0.449 | 1.00 | −0.73 |
| ATOM | 538 | CA | LEU | 105 | 5.865 | −6.735 | −1.854 | 1.00 | 0.36 |
| ATOM | 539 | C | LEU | 105 | 4.676 | −5.946 | −2.454 | 1.00 | 0.57 |
| ATOM | 540 | O | LEU | 105 | 3.525 | −6.101 | −2.047 | 1.00 | −0.57 |
| ATOM | 541 | CB | LEU | 105 | 5.992 | −8.015 | −2.715 | 1.00 | 0.00 |
| ATOM | 542 | CG | LEU | 105 | 7.333 | −8.772 | −2.748 | 1.00 | 0.00 |
| ATOM | 543 | CD1 | LEU | 105 | 8.507 | −7.866 | −3.103 | 1.00 | 0.00 |
| ATOM | 544 | CD2 | LEU | 105 | 7.617 | −9.584 | −1.490 | 1.00 | 0.00 |
| ATOM | 545 | N | VAL | 106 | 4.987 | −5.161 | −3.537 | 1.00 | −0.73 |
| ATOM | 546 | CA | VAL | 106 | 3.968 | −4.575 | −4.414 | 1.00 | 0.36 |
| ATOM | 547 | C | VAL | 106 | 4.187 | −5.219 | −5.797 | 1.00 | 0.57 |
| ATOM | 548 | O | VAL | 106 | 5.312 | −5.314 | −6.300 | 1.00 | −0.57 |
| ATOM | 549 | CB | VAL | 106 | 4.148 | −3.049 | −4.505 | 1.00 | 0.00 |
| ATOM | 550 | CG1 | VAL | 106 | 3.194 | −2.393 | −5.507 | 1.00 | 0.00 |
| ATOM | 551 | CG2 | VAL | 106 | 3.978 | −2.391 | −3.139 | 1.00 | 0.00 |
| ATOM | 552 | N | ALA | 107 | 3.042 | −5.637 | −6.430 | 1.00 | −0.73 |
| ATOM | 553 | CA | ALA | 107 | 3.072 | −6.236 | −7.753 | 1.00 | 0.36 |
| ATOM | 554 | C | ALA | 107 | 1.774 | −5.927 | −8.507 | 1.00 | 0.57 |
| ATOM | 555 | O | ALA | 107 | 0.702 | −5.717 | −7.943 | 1.00 | −0.57 |
| ATOM | 556 | CB | ALA | 107 | 3.257 | −7.745 | −7.658 | 1.00 | 0.00 |
| ATOM | 557 | N | GLU | 108 | 1.909 | −5.946 | −9.874 | 1.00 | −0.73 |
| ATOM | 558 | CA | GLU | 108 | 0.787 | −5.679 | −10.757 | 1.00 | 0.36 |
| ATOM | 559 | C | GLU | 108 | 0.416 | −7.015 | −11.447 | 1.00 | 0.57 |
| ATOM | 560 | O | GLU | 108 | 1.255 | −7.730 | −11.997 | 1.00 | −0.57 |
| ATOM | 561 | CB | GLU | 108 | 1.155 | −4.613 | −11.788 | 1.00 | 0.00 |
| ATOM | 562 | CG | GLU | 108 | 1.600 | −3.283 | −11.179 | 1.00 | −0.11 |
| ATOM | 563 | CD | GLU | 108 | 1.850 | −2.210 | −12.230 | 1.00 | 0.91 |
| ATOM | 564 | OE1 | GLU | 108 | 2.073 | −2.605 | −13.412 | 1.00 | −0.90 |
| ATOM | 565 | OE2 | GLU | 108 | 1.853 | −1.011 | −11.821 | 1.00 | −0.90 |
| ATOM | 566 | N | SER | 109 | −0.915 | −7.343 | −11.405 | 1.00 | −0.73 |
| ATOM | 567 | CA | SER | 109 | −1.476 | −8.564 | −11.970 | 1.00 | 0.36 |
| ATOM | 568 | C | SER | 109 | −2.793 | −8.227 | −12.698 | 1.00 | 0.57 |
| ATOM | 569 | O | SER | 109 | −3.836 | −7.978 | −12.093 | 1.00 | −0.57 |
| ATOM | 570 | CB | SER | 109 | −1.758 | −9.617 | −10.883 | 1.00 | 0.28 |
| ATOM | 571 | OG | SER | 109 | −2.479 | −9.097 | −9.758 | 1.00 | −0.68 |
| ATOM | 572 | N | THR | 110 | −2.684 | −8.191 | −14.076 | 1.00 | −0.73 |
| ATOM | 573 | GA | THR | 110 | −3.883 | −8.190 | −14.960 | 1.00 | 0.36 |
| ATOM | 574 | C | THR | 110 | −4.633 | −9.503 | −14.625 | 1.00 | 0.57 |
| ATOM | 575 | O | THR | 110 | −4.018 | −10.449 | −14.114 | 1.00 | −0.57 |
| ATOM | 576 | CB | THR | 110 | −3.399 | −8.155 | −16.436 | 1.00 | 0.28 |
| ATOM | 577 | OG1 | THR | 110 | −2.685 | −6.937 | −16.696 | 1.00 | −0.68 |
| ATOM | 578 | CG2 | THR | 110 | −4.443 | −8.284 | −17.532 | 1.00 | 0.00 |
| ATOM | 579 | N | PRO | 111 | −5.970 | −9.639 | −14.955 | 1.00 | −0.66 |
| ATOM | 580 | CA | PRO | 111 | −6.751 | −10.697 | −14.295 | 1.00 | 0.36 |
| ATOM | 581 | C | PRO | 111 | −6.345 | −12.183 | −14.352 | 1.00 | 0.57 |
| ATOM | 582 | O | PRO | 111 | −6.798 | −12.979 | −13.517 | 1.00 | −0.57 |
| ATOM | 583 | CB | PRO | 111 | −8.154 | −10.485 | −14.853 | 1.00 | 0.00 |
| ATOM | 584 | GG | PRO | 111 | −8.242 | −8.965 | −14.896 | 1.00 | 0.00 |
| ATOM | 585 | GD | PRO | 111 | −6.859 | −8.559 | −15.383 | 1.00 | 0.30 |
| ATOM | 586 | N | PRO | 112 | −5.505 | −12.636 | −15.338 | 1.00 | −0.66 |
| ATOM | 587 | GA | PRO | 112 | −4.779 | −13.906 | −15.198 | 1.00 | 0.36 |
| ATOM | 588 | C | PRO | 112 | −3.696 | −13.813 | −14.084 | 1.00 | 0.57 |
| ATOM | 589 | O | PRO | 112 | −2.488 | −13.855 | −14.311 | 1.00 | −0.57 |
| ATOM | 590 | CB | PRO | 112 | −4.159 | −14.131 | −16.587 | 1.00 | 0.00 |
| ATOM | 591 | CG | PRO | 112 | −4.999 | −13.260 | −17.514 | 1.00 | 0.00 |
| ATOM | 592 | GD | PRO | 112 | −5.303 | −12.054 | −16.645 | 1.00 | 0.30 |
| ATOM | 593 | N | GLY | 113 | −4.219 | −13.706 | −12.813 | 1.00 | −0.73 |
| ATOM | 594 | GA | GLY | 113 | −3.403 | −13.552 | −11.630 | 1.00 | 0.36 |
| ATOM | 595 | C | GLY | 113 | −2.926 | −14.885 | −11.040 | 1.00 | 0.57 |
| ATOM | 596 | O | GLY | 113 | −2.846 | −15.931 | −11.678 | 1.00 | −0.57 |
| ATOM | 597 | N | ASN | 114 | −2.501 | −14.782 | −9.729 | 1.00 | −0.73 |
| ATOM | 598 | CA | ASN | 114 | −1.859 | −15.910 | −9.041 | 1.00 | 0.36 |
| ATOM | 599 | C | ASN | 114 | −2.301 | −15.928 | −7.569 | 1.00 | 0.57 |
| ATOM | 600 | O | ASN | 114 | −1.576 | −16.274 | −6.637 | 1.00 | −0.57 |
| ATOM | 601 | CB | ASN | 114 | −0.343 | −15.817 | −9.172 | 1.00 | 0.06 |
| ATOM | 602 | CG | ASN | 114 | 0.332 | −17.158 | −8.998 | 1.00 | 0.57 |
| ATOM | 603 | OD1 | ASN | 114 | 0.759 | −17.832 | −9.930 | 1.00 | −0.57 |
| ATOM | 604 | ND2 | ASN | 114 | 0.493 | −17.583 | −7.713 | 1.00 | −0.80 |
| ATOM | 605 | N | ASP | 115 | −3.628 | −15.684 | −7.407 | 1.00 | −0.73 |
| ATOM | 606 | CA | ASP | 115 | −4.344 | −15.855 | −6.162 | 1.00 | 0.36 |
| ATOM | 607 | C | ASP | 115 | −5.518 | −16.865 | −6.403 | 1.00 | 0.57 |
| ATOM | 608 | O | ASP | 115 | −5.845 | −17.226 | −7.532 | 1.00 | −0.57 |
| ATOM | 609 | CB | ASP | 115 | −4.676 | −14.485 | −5.609 | 1.00 | −0.11 |
| ATOM | 610 | CG | ASP | 115 | −5.173 | −13.310 | −6.446 | 1.00 | 0.91 |
| ATOM | 611 | OD1 | ASP | 115 | −5.010 | −13.373 | −7.688 | 1.00 | −0.90 |
| ATOM | 612 | OD2 | ASP | 115 | −5.552 | −12.341 | −5.682 | 1.00 | −0.90 |
| ATOM | 613 | N | GLU | 116 | −6.178 | −17.303 | −5.264 | 1.00 | −0.73 |
| ATOM | 614 | CA | GLU | 116 | −7.407 | −18.113 | −5.319 | 1.00 | 0.36 |
| ATOM | 615 | C | GLU | 116 | −8.623 | −17.365 | −6.021 | 1.00 | 0.57 |
| ATOM | 616 | O | GLU | 116 | −9.458 | −18.013 | −6.667 | 1.00 | −0.57 |
| ATOM | 617 | CB | GLU | 116 | −7.917 | −18.515 | −3.907 | 1.00 | 0.00 |
| ATOM | 618 | CG | GLU | 116 | −7.023 | −19.412 | −3.053 | 1.00 | −0.11 |
| ATOM | 619 | CD | GLU | 116 | −5.913 | −18.713 | −2.296 | 1.00 | 0.91 |
| ATOM | 620 | OE1 | GLU | 116 | −5.794 | −18.988 | −1.061 | 1.00 | −0.90 |
| ATOM | 621 | OE2 | GLU | 116 | −5.123 | −17.979 | −2.948 | 1.00 | −0.90 |
| ATOM | 622 | N | PRO | 117 | −8.827 | −16.013 | −5.736 | 1.00 | −0.66 |
| ATOM | 623 | CA | PRO | 117 | −9.770 | −15.160 | −6.468 | 1.00 | 0.36 |
| ATOM | 624 | C | PRO | 117 | −9.256 | −14.844 | −7.907 | 1.00 | 0.57 |
| ATOM | 625 | O | PRO | 117 | −8.296 | −15.399 | −8.434 | 1.00 | −0.57 |
| ATOM | 626 | CB | PRO | 117 | −9.813 | −13.858 | −5.615 | 1.00 | 0.00 |
| ATOM | 627 | CG | PRO | 117 | −8.423 | −13.761 | −5.009 | 1.00 | 0.00 |
| ATOM | 628 | CD | PRO | 117 | −8.031 | −15.213 | −4.835 | 1.00 | 0.30 |
| ATOM | 629 | N | TYR | 118 | −10.000 | −13.892 | −8.575 | 1.00 | −0.73 |
| ATOM | 630 | CA | TYR | 118 | −9.483 | −13.201 | −9.759 | 1.00 | 0.36 |
| ATOM | 631 | C | TYR | 118 | −9.546 | −11.689 | −9.440 | 1.00 | 0.57 |
| ATOM | 632 | O | TYR | 118 | −10.397 | −11.223 | −8.679 | 1.00 | −0.57 |
| ATOM | 633 | CB | TYR | 118 | −10.257 | −13.586 | −11.036 | 1.00 | 0.14 |
| ATOM | 634 | CG | TYR | 118 | −11.702 | −13.126 | −11.105 | 1.00 | −0.14 |
| ATOM | 635 | CD1 | TYR | 118 | −12.077 | −12.147 | −12.035 | 1.00 | −0.15 |
| ATOM | 636 | CD2 | TYR | 118 | −12.684 | −13.633 | −10.240 | 1.00 | −0.15 |
| ATOM | 637 | CE1 | TYR | 118 | −13.373 | −11.630 | −12.043 | 1.00 | −0.15 |
| ATOM | 638 | CE2 | TYR | 118 | −13.978 | −13.099 | −10.236 | 1.00 | −0.15 |
| ATOM | 639 | CZ | TYR | 118 | −14.301 | −12.071 | −11.112 | 1.00 | 0.08 |
| ATOM | 640 | OH | TYR | 118 | −15.536 | −11.493 | −11.033 | 1.00 | −0.53 |
| ATOM | 641 | N | THR | 119 | −8.596 | −10.898 | −10.047 | 1.00 | −0.73 |
| ATOM | 642 | CA | THR | 119 | −8.747 | −9.440 | −10.073 | 1.00 | 0.36 |
| ATOM | 643 | C | THR | 119 | −9.629 | −9.098 | −11.293 | 1.00 | 0.57 |
| ATOM | 644 | O | THR | 119 | −9.782 | −9.868 | −12.242 | 1.00 | −0.57 |
| ATOM | 645 | CB | THR | 119 | −7.396 | −8.689 | −10.164 | 1.00 | 0.28 |
| ATOM | 646 | OG1 | THR | 119 | −6.566 | −9.255 | −11.185 | 1.00 | −0.68 |
| ATOM | 647 | CG2 | THR | 119 | −6.634 | −8.732 | −8.843 | 1.00 | 0.00 |
| ATOM | 648 | N | GLU | 120 | −10.229 | −7.861 | −11.256 | 1.00 | −0.73 |
| ATOM | 649 | CA | GLU | 120 | −10.865 | −7.276 | −12.439 | 1.00 | 0.36 |
| ATOM | 650 | C | GLU | 120 | −10.011 | −6.048 | −12.785 | 1.00 | 0.57 |
| ATOM | 651 | O | GLU | 120 | −9.128 | −5.645 | −12.026 | 1.00 | −0.57 |
| ATOM | 652 | CB | GLU | 120 | −12.318 | −6.878 | −12.162 | 1.00 | 0.00 |
| ATOM | 653 | CG | GLU | 120 | −13.199 | −8.114 | −12.026 | 1.00 | −0.11 |
| ATOM | 654 | CD | GLU | 120 | −14.694 | −7.888 | −11.819 | 1.00 | 0.91 |
| ATOM | 655 | OE1 | GLU | 120 | −15.086 | −6.689 | −11.807 | 1.00 | −0.90 |
| ATOM | 656 | OE2 | GLU | 120 | −15.367 | −8.960 | −11.702 | 1.00 | −0.90 |
| ATOM | 657 | N | GLN | 121 | −10.348 | −5.372 | −13.939 | 1.00 | −0.73 |
| ATOM | 658 | CA | GLN | 121 | −9.462 | −4.317 | −14.461 | 1.00 | 0.36 |
| ATOM | 659 | C | GLN | 121 | −9.203 | −3.246 | −13.374 | 1.00 | 0.57 |
| ATOM | 660 | O | GLN | 121 | −8.145 | −2.625 | −13.300 | 1.00 | −0.57 |
| ATOM | 661 | CB | GLN | 121 | −10.123 | −3.626 | −15.670 | 1.00 | 0.00 |
| ATOM | 662 | CG | GLN | 121 | −9.755 | −4.239 | −17.020 | 1.00 | 0.06 |
| ATOM | 663 | CD | GLN | 121 | −9.889 | −5.743 | −17.055 | 1.00 | 0.57 |
| ATOM | 664 | OE1 | GLN | 121 | −10.800 | −6.358 | −16.508 | 1.00 | −0.57 |
| ATOM | 665 | NE2 | GEN | 121 | −8.920 | −6.384 | −17.765 | 1.00 | −0.80 |
| ATOM | 666 | N | MET | 122 | −10.301 | −2.980 | −12.589 | 1.00 | −0.73 |
| ATOM | 667 | CA | MET | 122 | −10.319 | −2.004 | −11.519 | 1.00 | 0.36 |
| ATOM | 668 | C | MET | 122 | −10.509 | −2.603 | −10.101 | 1.00 | 0.57 |
| ATOM | 669 | O | MET | 122 | −11.023 | −1.941 | −9.199 | 1.00 | −0.57 |
| ATOM | 670 | CB | MET | 122 | −11.365 | −0.907 | −11.800 | 1.00 | 0.00 |
| ATOM | 671 | CG | MET | 122 | −12.821 | −1.396 | −11.790 | 1.00 | 0.23 |
| ATOM | 672 | SD | MET | 122 | −13.306 | −2.185 | −13.367 | 1.00 | −0.46 |
| ATOM | 673 | CE | MET | 122 | −14.492 | −3.401 | −12.722 | 1.00 | 0.23 |
| ATOM | 674 | N | GLY | 123 | −9.992 | −3.858 | −9.880 | 1.00 | −0.73 |
| ATOM | 675 | CA | GLY | 123 | −10.013 | −4.479 | −8.555 | 1.00 | 0.36 |
| ATOM | 676 | C | GLY | 123 | −8.630 | −4.975 | −8.114 | 1.00 | 0.57 |
| ATOM | 677 | O | GLY | 123 | −7.885 | −5.549 | −8.906 | 1.00 | −0.57 |
| ATOM | 678 | N | ASN | 124 | −8.335 | −4.749 | −6.787 | 1.00 | −0.73 |
| ATOM | 679 | CA | ASN | 124 | −7.002 | −4.958 | −6.198 | 1.00 | 0.36 |
| ATOM | 680 | C | ASN | 124 | −7.169 | −5.714 | −4.863 | 1.00 | 0.57 |
| ATOM | 681 | O | ASN | 124 | −8.268 | −5.844 | −4.317 | 1.00 | −0.57 |
| ATOM | 682 | CB | ASN | 124 | −6.342 | −3.630 | −5.853 | 1.00 | 0.06 |
| ATOM | 683 | CG | ASN | 124 | −6.192 | −2.760 | −7.060 | 1.00 | 0.57 |
| ATOM | 684 | OD1 | ASN | 124 | −5.779 | −3.175 | −8.142 | 1.00 | −0.57 |
| ATOM | 685 | ND2 | ASN | 124 | −6.522 | −1.456 | −6.863 | 1.00 | −0.80 |
| ATOM | 686 | N | CYS | 125 | −5.997 | −6.173 | −4.287 | 1.00 | −0.73 |
| ATOM | 687 | CA | GYS | 125 | −6.060 | −7.176 | −3.221 | 1.00 | 0.36 |
| ATOM | 688 | C | CYS | 125 | −4.725 | −7.301 | −2.434 | 1.00 | 0.57 |
| ATOM | 689 | O | CYS | 125 | −3.643 | −6.910 | −2.866 | 1.00 | −0.57 |
| ATOM | 690 | CB | CYS | 125 | −6.520 | −8.509 | −3.857 | 1.00 | 0.05 |
| ATOM | 691 | SG | CYS | 125 | −6.379 | −10.039 | −2.886 | 1.00 | −1.05 |
| ATOM | 692 | N | GLY | 126 | −4.891 | −7.951 | −1.216 | 1.00 | −0.73 |
| ATOM | 693 | CA | GLY | 126 | −3.791 | −8.567 | −0.486 | 1.00 | 0.36 |
| ATOM | 694 | C | GLY | 126 | −4.325 | −9.614 | 0.5 18 | 1.00 | 0.57 |
| ATOM | 695 | O | GLY | 126 | −4.442 | −9.388 | 1.721 | 1.00 | −0.57 |
| ATOM | 696 | N | GLU | 127 | −4.730 | −10.819 | −0.059 | 1.00 | −0.73 |
| ATOM | 697 | CA | GLU | 127 | −5.575 | −11.735 | 0.731 | 1.00 | 0.36 |
| ATOM | 698 | C | GLU | 127 | −4.873 | −12.419 | 1.930 | 1.00 | 0.57 |
| ATOM | 699 | O | GLU | 127 | −5.506 | −12.798 | 2.918 | 1.00 | −0.57 |
| ATOM | 700 | CB | GLU | 127 | −6.287 | −12.801 | −0.136 | 1.00 | 0.00 |
| ATOM | 701 | CG | GLU | 127 | −5.444 | −13.968 | −0.653 | 1.00 | −0.11 |
| ATOM | 702 | CD | GLU | 127 | −4.371 | −13.542 | −1.628 | 1.00 | 0.91 |
| ATOM | 703 | OE1 | GLU | 127 | −4.781 | −12.919 | −2.663 | 1.00 | −0.90 |
| ATOM | 704 | OE2 | GLU | 127 | −3.175 | −13.199 | −1.349 | 1.00 | −0.90 |
| ATOM | 705 | N | LYS | 128 | −3.542 | −12.726 | 1.765 | 1.00 | −0.73 |
| ATOM | 706 | CA | LYS | 128 | −2.769 | −13.379 | 2.813 | 1.00 | 0.36 |
| ATOM | 707 | C | LYS | 128 | −1.276 | −13.033 | 2.644 | 1.00 | 0.57 |
| ATOM | 708 | O | LYS | 128 | −0.645 | −13.221 | 1.605 | 1.00 | −0.57 |
| ATOM | 709 | CB | LYS | 128 | −2.989 | −14.903 | 2.86 | 61.00 | 0.00 |
| ATOM | 710 | CG | LYS | 128 | −2.527 | −15.611 | 1.593 | 1.00 | 0.00 |
| ATOM | 711 | CD | LYS | 128 | −3.183 | −16.971 | 1.371 | 1.00 | 0.00 |
| ATOM | 712 | CE | LYS | 128 | −2.913 | −17.410 | −0.062 | 1.00 | 0.50 |
| ATOM | 713 | NZ | LYS | 128 | −3.535 | −18.706 | −0.313 | 1.00 | −0.85 |
| ATOM | 714 | N | GLY | 129 | −0.695 | −12.487 | 3.774 | 1.00 | −0.73 |
| ATOM | 715 | CA | GLY | 129 | 0.554 | −11.760 | 3.644 | 1.00 | 0.36 |
| ATOM | 716 | C | GLY | 129 | 0.237 | −10.326 | 3.204 | 1.00 | 0.57 |
| ATOM | 717 | O | GLY | 129 | −0.606 | −10.059 | 2.349 | 1.00 | −0.57 |
| ATOM | 718 | N | GLU | 130 | 0.980 | −9.356 | 3.829 | 1.00 | −0.73 |
| ATOM | 719 | CA | GLU | 130 | 0.730 | −7.928 | 3.583 | 1.00 | 0.36 |
| ATOM | 720 | C | GLU | 130 | 1.478 | −7.563 | 2.290 | 1.00 | 0.57 |
| ATOM | 721 | O | GLU | 130 | 2.626 | −7.083 | 2.274 | 1.00 | −0.57 |
| ATOM | 722 | CB | GLU | 130 | 1.195 | −7.106 | 4.791 | 1.00 | 0.00 |
| ATOM | 723 | CG | GLU | 130 | 0.306 | −7.372 | 6.005 | 1.00 | −0.11 |
| ATOM | 724 | CD | GLU | 130 | 0.786 | −6.744 | 7.302 | 1.00 | 0.91 |
| ATOM | 725 | OE1 | GLU | 130 | 1.332 | −5.605 | 7.228 | 1.00 | −0.90 |
| ATOM | 726 | OE2 | GLU | 130 | 0.516 | −7.400 | 8.346 | 1.00 | −0.90 |
| ATOM | 727 | N | ARG | 131 | 0.852 | −7.963 | 1.133 | 1.00 | −0.73 |
| ATOM | 728 | CA | ARG | 131 | 1.433 | −7.779 | −0.187 | 1.00 | 0.36 |
| ATOM | 729 | C | ARG | 131 | 0.301 | −7.389 | −1.138 | 1.00 | 0.57 |
| ATOM | 730 | O | ARG | 131 | −0.835 | −7.840 | −1.014 | 1.00 | −0.57 |
| ATOM | 731 | CB | ARG | 131 | 2.137 | −9.040 | −0.719 | 1.00 | 0.00 |
| ATOM | 732 | CG | ARG | 131 | 2.905 | −9.781 | 0.369 | 1.00 | 0.00 |
| ATOM | 733 | CD | ARG | 131 | 3.859 | −10.843 | −0.146 | 1.00 | 0.33 |
| ATOM | 734 | NE | ARG | 131 | 4.712 | −11.291 | 0.963 | 1.00 | −0.84 |
| ATOM | 735 | CZ | ARG | 131 | 5.675 | −12.208 | 0.912 | 1.00 | 1.20 |
| ATOM | 736 | NH1 | ARG | 131 | 6.007 | −12.814 | −0.230 | 1.00 | −0.97 |
| ATOM | 737 | NH2 | ARG | 131 | 6.312 | −12.545 | 2.032 | 1.00 | −0.97 |
| ATOM | 738 | N | ILE | 132 | 0.667 | −6.524 | −2.141 | 1.00 | −0.73 |
| ATOM | 739 | CA | ILE | 132 | −0.347 | −5.811 | −2.909 | 1.00 | 0.36 |
| ATOM | 740 | C | ILE | 132 | −0.409 | −6.466 | −4.311 | 1.00 | 0.57 |
| ATOM | 741 | O | ILE | 132 | 0.548 | −6.472 | −5.087 | 1.00 | −0.57 |
| ATOM | 742 | CB | ILE | 132 | −0.035 | −4.299 | −3.015 | 1.00 | 0.00 |
| ATOM | 743 | CG1 | ILE | 132 | 0.471 | −3.667 | −1.697 | 1.00 | 0.00 |
| ATOM | 744 | CG2 | ILE | 132 | −1.261 | −3.538 | −3.537 | 1.00 | 0.00 |
| ATOM | 745 | CD1 | ILE | 132 | −0.457 | −3.800 | −0.500 | 1.00 | 0.00 |
| ATOM | 746 | N | HIS | 133 | −1.602 | −7.097 | 4.585 | 1.00 | −0.73 |
| ATOM | 747 | CA | HIS | 133 | −2.019 | −7.529 | −5.926 | 1.00 | 0.36 |
| ATOM | 748 | C | HIS | 133 | −2.793 | −6.322 | −6.538 | 1.00 | 0.57 |
| ATOM | 749 | O | HIS | 133 | −3.993 | −6.148 | −6.320 | 1.00 | −0.57 |
| ATOM | 750 | CB | HIS | 133 | −2.951 | −8.769 | −5.851 | 1.00 | 0.18 |
| ATOM | 751 | C | HIS | 133 | −2.301 | −10.043 | −5.384 | 1.0 | 0.05 |
| ATOM | 752 | N1 | HIS | 133 | −2.984 | −10.960 | −4.577 | 1.00 | −0.57 |
| ATOM | 753 | C1 | HIS | 133 | −2.103 | −11.918 | −4.362 | 1.00 | 0.04 |
| ATOM | 754 | N2 | HIS | 133 | −0.927 | −11.704 | −5.025 | 1.00 | 0.03 |
| ATOM | 755 | C2 | HIS | 133 | −1.031 | −10.510 | −5.680 | 1.00 | −0.30 |
| ATOM | 756 | N | LEU | 134 | −2.011 | −5.395 | −7.201 | 1.00 | −0.73 |
| ATOM | 757 | CA | LEU | 134 | −2.556 | −4.260 | −8.007 | 1.00 | 0.36 |
| ATOM | 758 | C | LEU | 134 | −2.785 | −4.787 | −9.453 | 1.00 | 0.57 |
| ATOM | 759 | O | LEU | 134 | −2.410 | −5.918 | −9.783 | 1.00 | −0.57 |
| ATOM | 760 | CB | LEU | 134 | −1.507 | −3.125 | −7.963 | 1.00 | 0.00 |
| ATOM | 761 | CG | LEU | 134 | −1.838 | −1.759 | −8.601 | 1.00 | 0.00 |
| ATOM | 762 | CD1 | LEU | 134 | −3.087 | −1.109 | −8.021 | 1.00 | 0.00 |
| ATOM | 763 | CD2 | LEU | 134 | −0.658 | −0.802 | −8.410 | 1.00 | 0.00 |
| ATOM | 764 | N | THR | 135 | −3.357 | −3.938 | −10.371 | 1.00 | −0.73 |
| ATOM | 765 | CA | THR | 135 | −3.445 | −4.268 | −11.803 | 1.00 | 0.36 |
| ATOM | 766 | C | THR | 135 | −2.584 | −3.275 | −12.638 | 1.00 | 0.57 |
| ATOM | 767 | O | THR | 135 | −2.481 | −2.082 | −12.332 | 1.00 | −0.57 |
| ATOM | 768 | CB | THR | 135 | −4.890 | −4.275 | −12.358 | 1.00 | 0.28 |
| ATOM | 769 | OG1 | THR | 135 | −5.416 | −2.949 | −12.435 | 1.00 | −0.68 |
| ATOM | 770 | CG2 | THR | 135 | −5.825 | −5.135 | −11.525 | 1.00 | 0.00 |
| ATOM | 771 | N | PRO | 136 | −2.006 | −3.747 | −13.806 | 1.00 | −0.66 |
| ATOM | 772 | GA | PRO | 136 | −1.388 | −2.840 | −14.787 | 1.00 | 0.36 |
| ATOM | 773 | C | PRO | 136 | −2.444 | −2.031 | −15.570 | 1.00 | 0.57 |
| ATOM | 774 | O | PRO | 136 | −2.149 | −1.075 | −16.285 | 1.00 | −0.57 |
| ATOM | 775 | CB | PRO | 136 | −0.690 | −3.766 | −15.795 | 1.00 | 0.00 |
| ATOM | 776 | CG | PRO | 136 | −0.535 | −5.086 | −15.066 | 1.00 | 0.00 |
| ATOM | 777 | CD | PRO | 136 | −1.735 | −5.127 | −14.138 | 1.00 | 0.30 |
| ATOM | 778 | N | ASP | 137 | −3.714 | −2.578 | −15.512 | 1.00 | −0.73 |
| ATOM | 779 | CA | ASP | 137 | −4.766 | −2.217 | −16.455 | 1.00 | 0.36 |
| ATOM | 780 | C | ASP | 137 | −5.275 | −0.776 | −16.199 | 1.00 | 0.57 |
| ATOM | 781 | O | ASP | 137 | −5.968 | −0.154 | −17.010 | 1.00 | −0.57 |
| ATOM | 782 | CB | ASP | 137 | −5.951 | −3.170 | −16.324 | 1.00 | −0.11 |
| ATOM | 783 | CG | ASP | 137 | −5.648 | −4.626 | −16.635 | 1.00 | 0.91 |
| ATOM | 784 | OD1 | ASP | 137 | −6.609 | −5.293 | −17.121 | 1.00 | −0.90 |
| ATOM | 785 | OD2 | ASP | 137 | −4.497 | −5.056 | −16.333 | 1.00 | −0.90 |
| ATOM | 786 | N | PHE | 138 | −4.977 | −0.259 | −14.956 | 1.00 | −0.73 |
| ATOM | 787 | CA | PHE | 138 | −5.417 | 1.062 | −14.556 | 1.00 | 0.36 |
| ATOM | 788 | C | PHE | 138 | −4.710 | 2.090 | −15.454 | 1.00 | 0.57 |
| ATOM | 789 | O | PHE | 138 | −3.488 | 2.231 | −15.504 | 1.00 | −0.57 |
| ATOM | 790 | CB | PHE | 138 | −5.013 | 1.451 | −13.127 | 1.00 | 0.14 |
| ATOM | 791 | CG | PHE | 138 | −5.926 | 0.912 | −12.064 | 1.00 | −0.14 |
| ATOM | 792 | CD1 | PHE | 138 | −7.200 | 1.457 | −11.851 | 1.00 | −0.15 |
| ATOM | 793 | CD2 | PHE | 138 | −5.503 | −0.146 | −11.264 | 1.00 | −0.15 |
| ATOM | 794 | GE1 | PHE | 138 | −8.015 | 0.981 | −10.824 | 1.00 | −0.15 |
| ATOM | 795 | GE2 | PHE | 138 | −6.340 | −0.650 | −10.276 | 1.00 | −0.15 |
| ATOM | 796 | GZ | PHE | 138 | −7.580 | −0.071 | −10.028 | 1.00 | −0.15 |
| ATOM | 797 | N | ILE | 139 | −5.572 | 2.898 | −16.182 | 1.00 | −0.73 |
| ATOM | 798 | CA | ILE | 139 | −5.001 | 3.912 | −17.071 | 1.00 | 0.36 |
| ATOM | 799 | C | ILE | 139 | −4.399 | 5.054 | −16.194 | 1.00 | 0.57 |
| ATOM | 800 | O | ILE | 139 | −5.016 | 6.070 | −15.865 | 1.00 | −0.57 |
| ATOM | 801 | CB | ILE | 139 | −6.019 | 4.471 | −18.098 | 1.00 | 0.00 |
| ATOM | 802 | CG1 | ILE | 139 | −7.368 | 4.903 | −17.484 | 1.00 | 0.00 |
| ATOM | 803 | CG2 | ILE | 139 | −6.237 | 3.446 | −19.220 | 1.00 | 0.00 |
| ATOM | 804 | CD1 | ILE | 139 | −8.191 | 5.768 | −18.432 | 1.00 | 0.00 |
| ATOM | 805 | N | ALA | 140 | −3.119 | 4.796 | −15.740 | 1.00 | −0.73 |
| ATOM | 806 | CA | ALA | 140 | −2.532 | 5.486 | −14.595 | 1.00 | 0.36 |
| ATOM | 807 | C | ALA | 140 | −2.036 | 6.897 | −14.981 | 1.00 | 0.57 |
| ATOM | 808 | O | ALA | 140 | −0.862 | 7.261 | −14.976 | 1.00 | −0.57 |
| ATOM | 809 | CB | ALA | 140 | −1.416 | 4.663 | −13.970 | 1.00 | 0.00 |
| ATOM | 810 | N | GLY | 141 | −3.061 | 7.767 | −15.292 | 1.00 | −0.73 |
| ATOM | 811 | CA | GLY | 141 | −2.816 | 9.129 | −15.663 | 1.00 | 0.36 |
| ATOM | 812 | C | GLY | 141 | −2.513 | 10.000 | −14.438 | 1.00 | 0.57 |
| ATOM | 813 | O | GLY | 141 | −2.660 | 9.662 | −13.268 | 1.00 | −0.57 |
| ATOM | 814 | N | LYS | 142 | −2.077 | 11.270 | −14.760 | 1.00 | −0.73 |
| ATOM | 815 | CA | LYS | 142 | −1.541 | 12.158 | −13.730 | 1.00 | 0.36 |
| ATOM | 816 | C | LYS | 142 | −2.579 | 13.134 | −13.115 | 1.00 | 0.57 |
| ATOM | 817 | O | LYS | 142 | −2.216 | 14.027 | −12.351 | 1.00 | −0.57 |
| ATOM | 818 | CB | LYS | 142 | −0.362 | 12.962 | −14.311 | 1.00 | 0.00 |
| ATOM | 819 | CG | LYS | 142 | 0.801 | 12.065 | −14.759 | 1.00 | 0.00 |
| ATOM | 820 | CD | LYS | 142 | 1.971 | 12.847 | −15.367 | 1.00 | 0.00 |
| ATOM | 821 | CE | LYS | 142 | 1.624 | 13.472 | −16.713 | 1.00 | 0.50 |
| ATOM | 822 | NZ | LYS | 142 | 2.840 | 14.082 | −17.302 | 1.00 | −0.85 |
| ATOM | 823 | N | LYS | 143 | −3.882 | 12.936 | −13.507 | 1.00 | −0.73 |
| ATOM | 824 | CA | LYS | 143 | −5.078 | 13.611 | −12.966 | 1.00 | 0.36 |
| ATOM | 825 | C | LYS | 143 | −6.143 | 13.675 | −14.081 | 1.00 | 0.57 |
| ATOM | 826 | O | LYS | 143 | −7.345 | 13.707 | −13.837 | 1.00 | −0.57 |
| ATOM | 827 | CB | LYS | 143 | −4.881 | 15.047 | −12.446 | 1.00 | 0.00 |
| ATOM | 828 | CG | LYS | 143 | −4.670 | 15.117 | −10.925 | 1.00 | 0.00 |
| ATOM | 829 | CD | LYS | 143 | −5.992 | 15.034 | −10.149 | 1.00 | 0.00 |
| ATOM | 830 | CE | LYS | 143 | −5.813 | 15.020 | −8.636 | 1.00 | 0.50 |
| ATOM | 831 | NZ | LYS | 143 | −5.190 | 16.263 | −8.160 | 1.00 | −0.85 |
| ATOM | 832 | N | LEU | 144 | −5.629 | 13.839 | −15.363 | 1.00 | −0.73 |
| ATOM | 833 | CA | LEU | 144 | −6.539 | 14.162 | −16.472 | 1.00 | 0.36 |
| ATOM | 834 | C | LEU | 144 | −7.448 | 12.958 | −16.807 | 1.00 | 0.57 |
| ATOM | 835 | O | LEU | 144 | −8.552 | 13.082 | −17.325 | 1.00 | −0.57 |
| ATOM | 836 | CE | LEU | 144 | −5.799 | 14.658 | −17.728 | 1.00 | 0.00 |
| ATOM | 837 | CG | LEU | 144 | −4.949 | 13.637 | −18.521 | 1.00 | 0.00 |
| ATOM | 838 | CD1 | LEU | 144 | −4.529 | 14.254 | −19.861 | 1.00 | 0.00 |
| ATOM | 839 | CD2 | LEU | 144 | −3.702 | 13.174 | −17.769 | 1.00 | 0.00 |
| ATOM | 840 | N | ALA | 145 | −6.847 | 11.729 | −16.594 | 1.00 | −0.73 |
| ATOM | 841 | CA | ALA | 145 | −7.676 | 10.544 | −16.453 | 1.00 | 0.36 |
| ATOM | 842 | C | ALA | 145 | −7.809 | 10.313 | −14.936 | 1.00 | 0.57 |
| ATOM | 843 | O | ALA | 145 | −6.914 | 10.623 | −14.146 | 1.00 | −0.57 |
| ATOM | 844 | CB | ALA | 145 | −7.024 | 9.332 | −17.095 | 1.00 | 0.00 |
| ATOM | 845 | N | GLU | 146 | −8.958 | 9.648 | −14.554 | 1.00 | −0.73 |
| ATOM | 846 | CA | GLU | 146 | −9.336 | 9.618 | −13.136 | 1.00 | 0.36 |
| ATOM | 847 | C | GLU | 146 | −8.305 | 8.849 | −12.280 | 1.00 | 0.57 |
| ATOM | 848 | O | GLU | 146 | −8.081 | 9.127 | −11.100 | 1.00 | −0.57 |
| ATOM | 849 | CB | GLU | 146 | −10.701 | 8.913 | −12.993 | 1.00 | 0.00 |
| ATOM | 850 | CG | GLU | 146 | −11.859 | 9.908 | −12.927 | 1.00 | −0.11 |
| ATOM | 851 | CD | GLU | 146 | −11.968 | 10.634 | −11.596 | 1.00 | 0.91 |
| ATOM | 852 | OE1 | GLU | 146 | −11.378 | 10.115 | −10.607 | 1.00 | −0.90 |
| ATOM | 853 | OE2 | GLU | 146 | −12.680 | 11.682 | −11.599 | 1.00 | −0.90 |
| ATOM | 854 | N | TYR | 147 | −7.799 | 7.724 | −12.878 | 1.00 | −0.73 |
| ATOM | 855 | CA | TYR | 147 | −7.143 | 6.634 | −12.158 | 1.00 | 0.36 |
| ATOM | 856 | C | TYR | 147 | −5.641 | 6.861 | −11.930 | 1.00 | 0.57 |
| ATOM | 857 | O | TYR | 147 | 4.813 | 5.961 | −12.032 | 1.00 | −0.57 |
| ATOM | 858 | CB | TYR | 147 | −7.391 | 5.283 | −12.845 | 1.00 | 0.14 |
| ATOM | 859 | CG | TYR | 147 | −8.855 | 4.907 | −12.779 | 1.00 | −0.14 |
| ATOM | 860 | CD1 | TYR | 147 | −9.686 | 5.067 | −13.896 | 1.00 | −0.15 |
| ATOM | 861 | CD2 | TYR | 147 | −9.402 | 4.428 | −11.579 | 1.00 | −0.15 |
| ATOM | 862 | CE1 | TYR | 147 | −11.047 | 4.769 | −13.808 | 1.00 | −0.15 |
| ATOM | 863 | CE2 | TYR | 147 | −10.760 | 4.132 | −11.493 | 1.00 | −0.15 |
| ATOM | 864 | CZ | TYR | 147 | −11.572 | 4.310 | −12.605 | 1.00 | 0.08 |
| ATOM | 865 | OH | TYR | 147 | −12.896 | 4.019 | −12.474 | 1.00 | −0.53 |
| ATOM | 866 | N | GLY | 148 | −5.314 | 8.092 | −11.396 | 1.00 | −0.73 |
| ATOM | 867 | CA | GLY | 148 | −3.987 | 8.309 | −10.845 | 1.00 | 0.36 |
| ATOM | 868 | C | GLY | 148 | −3.921 | 7.601 | −9.486 | 1.00 | 0.57 |
| ATOM | 869 | O | GLY | 148 | −4.676 | 7.921 | −8.558 | 1.00 | −0.57 |
| ATOM | 870 | N | PRO | 149 | −3.050 | 6.546 | −9.340 | 1.00 | −0.66 |
| ATOM | 871 | CA | PRO | 149 | −3.351 | 5.465 | −8.396 | 1.00 | 0.36 |
| ATOM | 872 | C | PRO | 149 | −2.915 | 5.739 | −6.952 | 1.00 | 0.57 |
| ATOM | 873 | O | PRO | 149 | −2.793 | 4.826 | −6.143 | 1.00 | −0.57 |
| ATOM | 874 | CB | PRO | 149 | −2.580 | 4.254 | −8.945 | 1.00 | 0.00 |
| ATOM | 815 | CG | PRO | 149 | −1.388 | 4.908 | −9.633 | 1.00 | 0.00 |
| ATOM | 876 | CD | PRO | 149 | −2.029 | 6.119 | −10.288 | 1.00 | 0.30 |
| ATOM | 877 | N | GLN | 150 | −2.755 | 7.055 | −6.580 | 1.00 | −0.73 |
| ATOM | 878 | CA | GLN | 150 | −2.458 | 7.373 | −5.186 | 1.00 | 0.36 |
| ATOM | 879 | C | GLN | 150 | −3.759 | 7.502 | −4.371 | 1.00 | 0.57 |
| ATOM | 880 | O | GLN | 150 | −3.936 | 6.901 | −3.317 | 1.00 | −0.57 |
| ATOM | 881 | CB | GLN | 150 | −1.636 | 8.664 | −5.103 | 1.00 | 0.00 |
| ATOM | 882 | CG | GLN | 150 | −1.084 | 8.911 | −3.696 | 1.00 | 0.06 |
| ATOM | 883 | CD | GLN | 150 | −0.525 | 10.309 | −3.543 | 1.00 | 0.57 |
| ATOM | 884 | OE1 | GLN | 150 | −0.724 | 11.234 | −4.323 | 1.00 | −0.57 |
| ATOM | 885 | NE2 | GLN | 150 | 0.233 | 10.500 | −2.430 | 1.00 | −0.80 |
| ATOM | 886 | N | GLY | 151 | −4.647 | 8.450 | −4.825 | 1.00 | −0.73 |
| ATOM | 887 | CA | GLY | 151 | −5.822 | 8.799 | 4.048 | 1.00 | 0.36 |
| ATOM | 888 | C | GLY | 151 | −7.071 | 8.044 | −4.488 | 1.00 | 0.57 |
| ATOM | 889 | O | GLY | 151 | −8.133 | 8.644 | 4.658 | 1.00 | −0.57 |
| ATOM | 890 | N | LYS | 152 | −6.911 | 6.681 | −4.619 | 1.00 | −0.73 |
| ATOM | 891 | CA | LYS | 152 | −8.050 | 5.807 | −4.923 | 1.00 | 0.36 |
| ATOM | 892 | C | LYS | 152 | −7.655 | 4.335 | −4.981 | 1.00 | 0.57 |
| ATOM | 893 | O | LYS | 152 | −8.458 | 3.459 | 4.687 | 1.00 | −0.57 |
| ATOM | 894 | CB | LYS | 152 | −8.750 | 6.146 | −6.244 | 1.00 | 0.00 |
| ATOM | 895 | CG | LYS | 152 | −7.810 | 6.348 | −7.442 | 1.00 | 0.00 |
| ATOM | 896 | CD | LYS | 152 | −8.124 | 1.649 | −8.168 | 1.00 | 0.00 |
| ATOM | 897 | CE | LYS | 152 | −9.515 | 7.683 | −8.791 | 1.00 | 0.50 |
| ATOM | 898 | NZ | LYS | 152 | −9.807 | 9.082 | −9.095 | 1.00 | −0.85 |
| ATOM | 899 | N | ALA | 153 | −6.418 | 4.079 | −5.554 | 1.00 | −0.73 |
| ATOM | 900 | CA | ALA | 153 | −5.776 | 2.826 | −5.155 | 1.00 | 0.36 |
| ATOM | 901 | C | ALA | 153 | 4.914 | 3.261 | −3.950 | 1.00 | 0.57 |
| ATOM | 902 | O | ALA | 153 | −5.435 | 3.788 | −2.956 | 1.00 | −0.57 |
| ATOM | 903 | CB | ALA | 153 | −5.063 | 2.171 | −6.325 | 1.00 | 0.00 |
| ATOM | 904 | N | PHE | 154 | −3.551 | 3.374 | −4.098 | 1.00 | −0.73 |
| ATOM | 905 | CA | PHE | 154 | −2.641 | 2.863 | −3.070 | 1.00 | 0.36 |
| ATOM | 906 | C | PHE | 154 | −2.926 | 3.215 | −1.595 | 1.00 | 0.57 |
| ATOM | 907 | O | PHE | 154 | −2.465 | 2.537 | −0.674 | 1.00 | −0.57 |
| ATOM | 908 | CB | PHE | 154 | −1.220 | 3.410 | −3.315 | 1.00 | 0.14 |
| ATOM | 909 | CG | PHE | 154 | −0.412 | 2.748 | −4.406 | 1.00 | −0.14 |
| ATOM | 910 | CD1 | PHE | 154 | 0.032 | 1.430 | −4.252 | 1.00 | −0.15 |
| ATOM | 911 | GD2 | PHE | 154 | −0.042 | 3.455 | −5.558 | 1.00 | −0.15 |
| ATOM | 912 | CE1 | PHE | 154 | 0.828 | 0.835 | −5.231 | 1.00 | −0.15 |
| ATOM | 913 | CE2 | PHE | 154 | 0.740 | 2.853 | −6.545 | 1.00 | −0.15 |
| ATOM | 914 | CZ | PHE | 154 | 1.182 | 1.545 | −6.376 | 1.00 | −0.15 |
| ATOM | 915 | N | VAL | 155 | −3.543 | 4.420 | −1.343 | 1.00 | −0.73 |
| ATOM | 916 | CA | VAL | 155 | −3.933 | 4.730 | 0.030 | 1.00 | 0.36 |
| ATOM | 917 | C | VAL | 155 | −5.041 | 3.765 | 0.518 | 1.00 | 0.57 |
| ATOM | 918 | O | VAL | 155 | −5.102 | 3.438 | 1.704 | 1.00 | −0.57 |
| ATOM | 919 | CB | VAL | 155 | −4.356 | 6.203 | 0.180 | 1.00 | 0.00 |
| ATOM | 920 | CG1 | VAL | 155 | −4.799 | 6.513 | 1.614 | 1.00 | 0.00 |
| ATOM | 921 | CG2 | VAL | 155 | −3.193 | 7.139 | −0.161 | 1.00 | 0.00 |
| ATOM | 922 | N | HIS | 156 | −5.965 | 3.371 | −0.416 | 1.00 | −0.73 |
| ATOM | 923 | CA | HIS | 156 | −7.024 | 2.377 | −0.148 | 1.00 | 0.36 |
| ATOM | 924 | C | HIS | 156 | −6.318 | 1.029 | 0.189 | 1.00 | 0.57 |
| ATOM | 925 | O | HIS | 156 | −6.637 | 0.333 | 1.163 | 1.00 | −0.57 |
| ATOM | 926 | CB | HIS | 156 | −7.957 | 2.312 | −1.356 | 1.00 | 0.17 |
| ATOM | 927 | C | HIS | 156 | −9.225 | 1.625 | −1.068 | 1.00 | −0.02 |
| ATOM | 928 | N1 | HIS | 156 | −10.157 | 1.265 | −2.008 | 1.00 | −1.30 |
| ATOM | 929 | C1 | HIS | 156 | −11.195 | 0.660 | −1.349 | 1.00 | 0.14 |
| ATOM | 930 | N2 | HIS | 156 | −11.053 | 0.545 | −0.028 | 1.00 | −0.28 |
| ATOM | 931 | C2 | HIS | 156 | −9.804 | 1.095 | 0.141 | 1.00 | −0.01 |
| ATOM | 932 | N | GLU | 157 | −5.281 | 0.648 | −0.635 | 1.00 | −0.73 |
| ATOM | 933 | CA | GLU | 157 | −4.667 | −0.669 | −0.489 | 1.00 | 0.36 |
| ATOM | 934 | C | GLU | 157 | −3.845 | −0.660 | 0.803 | 1.00 | 0.57 |
| ATOM | 935 | O | GLU | 157 | −3.903 | −1.579 | 1.617 | 1.00 | −0.57 |
| ATOM | 936 | CB | GLU | 157 | −3.740 | −1.053 | −1.652 | 1.00 | 0.00 |
| ATOM | 937 | CG | GLU | 157 | −4.514 | −1.536 | −2.880 | 1.00 | −0.11 |
| ATOM | 938 | CD | GLU | 157 | −5.273 | −0.419 | −3.565 | 1.00 | 0.91 |
| ATOM | 939 | OE1 | GLU | 157 | −6.075 | −0.764 | −4.463 | 1.00 | −0.90 |
| ATOM | 940 | OE2 | GLU | 157 | −4.996 | 0.753 | −3.178 | 1.00 | −0.90 |
| ATOM | 941 | N | TRP | 158 | −2.994 | 0.405 | 0.982 | 1.00 | −0.73 |
| ATOM | 942 | CA | TRP | 158 | −2.187 | 0.501 | 2.190 | 1.00 | 0.36 |
| ATOM | 943 | C | TRP | 158 | −3.008 | 0.928 | 3.427 | 1.00 | 0.57 |
| ATOM | 944 | O | TRP | 158 | −2.527 | 0.916 | 4.563 | 1.00 | −0.57 |
| ATOM | 945 | CB | TRP | 158 | −0.946 | 1.390 | 2.021 | 1.00 | 0.18 |
| ATOM | 946 | CG | TRP | 158 | 0.117 | 0.704 | 1.205 | 1.00 | −0.18 |
| ATOM | 947 | CD1 | TRP | 158 | 0.295 | 0.789 | −0.161 | 1.00 | −0.30 |
| ATOM | 948 | CD2 | TRP | 158 | 1.106 | −0.213 | 1.689 | 1.00 | 0.00 |
| ATOM | 949 | NE1 | TRP | 158 | 1.289 | −0.076 | −0.522 | 1.00 | 0.03 |
| ATOM | 950 | CE2 | TRP | 158 | 1.780 | −0.727 | 0.584 | 1.00 | −0.15 |
| ATOM | 951 | CE3 | TRP | 158 | 1.468 | −0.685 | 2.964 | 1.00 | −0.15 |
| ATOM | 952 | CZ2 | TRP | 158 | 2.754 | −1.729 | 0.688 | 1.00 | −0.15 |
| ATOM | 953 | CZ3 | TRP | 158 | 2.448 | −1.675 | 3.091 | 1.00 | −0.15 |
| ATOM | 954 | CH2 | TRP | 158 | 3.072 | −2.198 | 1.964 | 1.00 | −0.15 |
| ATOM | 955 | N | ALA | 159 | −4.324 | 1.266 | 3.234 | 1.00 | −0.73 |
| ATOM | 956 | CA | ALA | 159 | −5.227 | 1.389 | 4.360 | 1.00 | 0.36 |
| ATOM | 957 | C | ALA | 159 | −5.568 | −0.046 | 4.802 | 1.00 | 0.57 |
| ATOM | 958 | O | ALA | 159 | −5.510 | −0.399 | 5.984 | 1.00 | −0.57 |
| ATOM | 959 | CB | ALA | 159 | −6.490 | 2.176 | 4.064 | 1.00 | 0.00 |
| ATOM | 960 | N | HIS | 160 | −5.984 | −0.920 | 3.831 | 1.00 | −0.73 |
| ATOM | 961 | CA | HIS | 160 | −6.401 | −2.274 | 4.186 | 1.00 | 0.36 |
| ATOM | 962 | C | HIS | 160 | −5.236 | −3.221 | 4.480 | 1.00 | 0.57 |
| ATOM | 963 | O | HIS | 160 | −5.253 | −3.954 | 5.464 | 1.00 | −0.57 |
| ATOM | 964 | CB | HIS | 160 | −7.271 | −2.949 | 3.124 | 1.00 | 0.18 |
| ATOM | 965 | CG | HIS | 160 | −8.551 | −2.235 | 2.939 | 1.00 | −0.33 |
| ATOM | 966 | ND1 | HIS | 160 | −9.463 | −2.000 | 3.928 | 1.00 | 0.03 |
| ATOM | 967 | CD2 | HIS | 160 | −9.040 | −1.645 | 1.801 | 1.00 | 0.08 |
| ATOM | 968 | CE1 | HIS | 160 | −10.417 | −1.235 | 3.327 | 1.00 | 0.04 |
| ATOM | 969 | NE2 | HIS | 160 | −10.255 | −1.086 | 2.026 | 1.00 | −0.57 |
| ATOM | 970 | N | LEU | 161 | −4.284 | −3.314 | 3.514 | 1.00 | −0.73 |
| ATOM | 971 | CA | LEU | 161 | −3.443 | −4.490 | 3.302 | 1.00 | 0.36 |
| ATOM | 972 | C | LEU | 161 | −2.067 | −4.346 | 3.982 | 1.00 | 0.57 |
| ATOM | 973 | O | LEU | 161 | −1.006 | −4.576 | 3.404 | 1.00 | −0.57 |
| ATOM | 974 | CB | LEU | 161 | −3.274 | −4.718 | 1.796 | 1.00 | 0.00 |
| ATOM | 975 | CG | LEU | 161 | −4.589 | −4.713 | 0.994 | 1.00 | 0.00 |
| ATOM | 976 | CD1 | LEU | 161 | −4.267 | −4.768 | −0.490 | 1.00 | 0.00 |
| ATOM | 977 | CD2 | LEU | 161 | −5.536 | −5.829 | 1.426 | 1.00 | 0.00 |
| ATOM | 978 | N | ARG | 162 | −2.143 | −4.036 | 5.318 | 1.00 | −0.73 |
| ATOM | 979 | CA | ARG | 162 | −0.992 | −3.563 | 6.088 | 1.00 | 0.36 |
| ATOM | 980 | C | ARG | 162 | −1.357 | −3.788 | 7.576 | 1.00 | 0.57 |
| ATOM | 981 | O | ARG | 162 | −2.523 | −3.784 | 7.978 | 1.00 | −0.57 |
| ATOM | 982 | CB | ARG | 162 | −0.753 | −2.094 | 5.705 | 1.00 | 0.00 |
| ATOM | 983 | CG | ARG | 162 | 0.140 | −1.237 | 6.616 | 1.00 | 0.00 |
| ATOM | 984 | CD | ARG | 162 | −0.335 | 0.223 | 6.628 | 1.00 | 0.33 |
| ATOM | 985 | NE | ARG | 162 | −1.775 | 0.327 | 6.909 | 1.00 | −0.84 |
| ATOM | 986 | CZ | ARG | 162 | −2.386 | −0.262 | 7.938 | 1.00 | 1.20 |
| ATOM | 987 | NH1 | ARG | 162 | −1.733 | −0.598 | 9.039 | 1.00 | −0.97 |
| ATOM | 988 | NH2 | ARG | 162 | −3.682 | −0.53 | 87.879 | 1.00 | −0.97 |
| ATOM | 989 | N | TRP | 163 | −0.283 | −3.910 | 8.430 | 1.00 | −0.73 |
| ATOM | 990 | CA | TRP | 163 | −0.424 | −4.477 | 9.783 | 1.00 | 0.36 |
| ATOM | 991 | C | TRP | 163 | −1.468 | −3.695 | 10.606 | 1.00 | 0.57 |
| ATOM | 992 | O | TRP | 163 | −1.378 | −2.495 | 10.876 | 1.00 | −0.57 |
| ATOM | 993 | CB | TRP | 163 | 0.926 | −4.415 | 10.522 | 1.00 | 0.18 |
| ATOM | 994 | CG | TRP | 163 | 0.856 | −4.832 | 11.963 | 1.00 | −0.18 |
| ATOM | 995 | CD1 | TRP | 163 | 1.053 | −4.024 | 13.069 | 1.00 | −0.30 |
| ATOM | 996 | CD2 | TRP | 163 | 0.606 | −6.154 | 12.454 | 1.00 | 0.00 |
| ATOM | 997 | NE1 | TRP | 163 | 0.928 | −4.799 | 14.193 | 1.00 | 0.03 |
| ATOM | 998 | CE2 | TRP | 163 | 0.625 | −6.093 | 13.846 | 1.00 | −0.15 |
| ATOM | 999 | CE3 | TRP | 163 | 0.390 | −7.405 | 11.842 | 1.00 | −0.15 |
| ATOM | 1000 | CZ2 | TRP | 163 | 0.418 | −7.213 | 14.658 | 1.00 | −0.15 |
| ATOM | 1001 | CZ3 | TRP | 163 | 0.190 | −8.538 | 12.638 | 1.00 | −0.15 |
| ATOM | 1002 | CE2 | TRP | 163 | 0.199 | −8.440 | 14.026 | 1.00 | −0.15 |
| ATOM | 1003 | N | GLY | 164 | −2.583 | −4.417 | 10.980 | 1.00 | −0.73 |
| ATOM | 1004 | CA | GLY | 164 | −3.727 | −3.746 | 11.564 | 1.00 | 0.36 |
| ATOM | 1005 | C | GLY | 164 | −4.541 | −3.077 | 10.453 | 1.00 | 0.57 |
| ATOM | 1006 | O | GLY | 164 | −4.304 | −1.941 | 10.037 | 1.00 | −0.57 |
| ATOM | 1007 | N | VAL | 165 | −5.500 | −3.895 | 9.898 | 1.00 | −0.73 |
| ATOM | 1008 | CA | VAL | 165 | −6.197 | −3.515 | 8.667 | l.00 | 0.36 |
| ATOM | 1009 | C | VAL | 165 | −7.250 | −2.439 | 9.016 | 1.00 | 0.57 |
| ATOM | 1010 | O | VAL | 165 | −7.829 | −2.427 | 10.105 | 1.00 | −0.57 |
| ATOM | 1011 | CB | VAL | 165 | −6.872 | −4.724 | 7.973 | 1.00 | 0.00 |
| ATOM | 1012 | CG1 | VAL | 165 | −5.869 | −5.856 | 7.702 | 1.00 | 0.00 |
| ATOM | 1013 | CG2 | VAL | 165 | −8.066 | −5.295 | 8.747 | 1.00 | 0.00 |
| ATOM | 1014 | N | PHE | 166 | −7.532 | −1.536 | 8.018 | 1.00 | −0.73 |
| ATOM | 1015 | CA | PHE | 166 | −8.670 | −0.622 | 8.120 | 1.00 | 0.36 |
| ATOM | 1016 | C | PHE | 166 | −9.874 | −1.186 | 7.348 | 1.00 | 0.57 |
| ATOM | 1017 | O | PHE | 166 | −9.771 | −2.065 | 6.496 | 1.00 | −0.57 |
| ATOM | 1018 | CB | PHE | 166 | −8.348 | 0.788 | 7.616 | 1.00 | 0.14 |
| ATOM | 1019 | CG | PHE | 166 | −7.530 | 1.551 | 8.627 | 1.00 | −0.14 |
| ATOM | 1020 | CD1 | PHE | 166 | −8.150 | 2.089 | 9.757 | 1.00 | −0.15 |
| ATOM | 1021 | CD2 | PHE | 166 | −6.141 | 1.650 | 8.505 | 1.00 | −0.15 |
| ATOM | 1022 | CE1 | PHE | 166 | −1.386 | 2.671 | 10.762 | 1.00 | −0.15 |
| ATOM | 1023 | CE2 | PHE | 166 | −5.374 | 2.198 | 9.528 | 1.00 | −0.15 |
| ATOM | 1024 | CZ | PHE | 166 | −5.998 | 2.696 | 10.662 | 1.00 | −0.15 |
| ATOM | 1025 | N | ASP | 167 | −11.070 | −0.608 | 7.695 | 1.00 | −0.73 |
| ATOM | 1026 | CA | ASP | 167 | −12.379 | −1.138 | 7.309 | 1.00 | 0.36 |
| ATOM | 1027 | C | ASP | 167 | −12.842 | −0.443 | 6.010 | 1.00 | 0.57 |
| ATOM | 1028 | O | ASP | 167 | −12.260 | 0.534 | 5.529 | 1.00 | −0.57 |
| ATOM | 1029 | CB | ASP | 167 | −13.439 | −0.884 | 8.380 | 1.00 | −0.11 |
| ATOM | 1030 | CG | ASP | 167 | −12.867 | −1.127 | 9.739 | 1.00 | 0.91 |
| ATOM | 1031 | OD1 | ASP | 167 | −13.206 | −2.166 | 10.370 | 1.00 | −0.90 |
| ATOM | 1032 | OD2 | ASP | 167 | −12.021 | −0.283 | 10.181 | 1.00 | −0.90 |
| ATOM | 1033 | N | GLU | 168 | −13.975 | −0.990 | 5.424 | 1.00 | −0.73 |
| ATOM | 1034 | CA | GLU | 168 | −14.753 | −0.178 | 4.489 | 1.00 | 0.36 |
| ATOM | 1035 | C | GLU | 168 | −15.725 | 0.685 | 5.344 | 1.00 | 0.57 |
| ATOM | 1036 | O | GLU | 168 | −15.896 | 0.517 | 6.551 | 1.00 | −0.57 |
| ATOM | 1037 | CB | GLU | 168 | −15.594 | −1.024 | 3.502 | 1.00 | 0.00 |
| ATOM | 1038 | CG | GLU | 168 | −14.862 | −2.199 | 2.835 | 1.00 | −0.11 |
| ATOM | 1039 | CD | GLU | 168 | −13.747 | −1.826 | 1.903 | 1.00 | 0.91 |
| ATOM | 1040 | OE1 | GLU | 168 | −13.002 | −2.706 | 1.388 | 1.00 | −0.90 |
| ATOM | 1041 | OE2 | GLU | 168 | −13.419 | −0.647 | 1.614 | 1.00 | −0.90 |
| ATOM | 1042 | N | TYR | 169 | −16.431 | 1.621 | 4.636 | 1.00 | −0.73 |
| ATOM | 1043 | CA | TYR | 169 | −17.284 | 2.629 | 5.263 | 1.00 | 0.36 |
| ATOM | 1044 | C | TYR | 169 | −18.298 | 3.072 | 4.179 | 1.00 | 0.57 |
| ATOM | 1045 | O | TYR | 169 | −18.420 | 2.478 | 3.105 | 1.00 | −0.57 |
| ATOM | 1046 | CB | TYR | 169 | −16.422 | 3.788 | 5.796 | 1.00 | 0.14 |
| ATOM | 1047 | CG | TYR | 169 | −17.049 | 4.592 | 6.912 | 1.00 | −0.14 |
| ATOM | 1048 | CD1 | TYR | 169 | −17.330 | 3.988 | 8.147 | 1.00 | −0.15 |
| ATOM | 1049 | CD2 | TYR | 169 | −17.326 | 5.956 | 6.740 | 1.00 | −0.15 |
| ATOM | 1050 | CE1 | TYR | 169 | −17.894 | 4.728 | 9.188 | 1.00 | −0.15 |
| ATOM | 1051 | CE2 | TYR | 169 | −17.894 | 6.693 | 7.780 | 1.00 | −0.15 |
| ATOM | 1052 | CZ | TYR | 169 | −18.170 | 6.076 | 8.994 | 1.00 | 0.08 |
| ATOM | 1053 | OH | TYR | 169 | −18.713 | 6.842 | 9.982 | 1.00 | −0.53 |
| ATOM | 1054 | N | ASN | 170 | −19.131 | 4.121 | 4.496 | 1.00 | −0.73 |
| ATOM | 1055 | CA | ASN | 170 | −20.255 | 4.494 | 3.622 | 1.00 | 0.36 |
| ATOM | 1056 | C | ASN | 170 | −20.494 | 6.016 | 3.728 | 1.00 | 0.57 |
| ATOM | 1057 | O | ASN | 170 | −21.603 | 6.529 | 3.855 | 1.00 | −0.57 |
| ATOM | 1058 | CB | ASN | 170 | −21.500 | 3.663 | 3.949 | 1.00 | 0.06 |
| ATOM | 1059 | CG | ASN | 170 | −21.891 | 2.749 | 2.808 | 1.00 | 0.57 |
| ATOM | 1060 | OD1 | ASN | 170 | −22.969 | 2.831 | 2.226 | 1.00 | −0.57 |
| ATOM | 1061 | ND2 | ASN | 170 | −21.004 | 1.766 | 2.491 | 1.00 | −0.80 |
| ATOM | 1062 | N | ASN | 171 | −19.351 | 6.776 | 3.571 | 1.00 | −0.73 |
| ATOM | 1063 | CA | ASN | 171 | −19.393 | 8.238 | 3.396 | 1.00 | 0.36 |
| ATOM | 1064 | C | ASN | 171 | −18.011 | 8.671 | 2.832 | 1.00 | 0.57 |
| ATOM | 1065 | O | ASN | 171 | −17.013 | 7.962 | 2.966 | 1.00 | −0.57 |
| ATOM | 1066 | CB | ASN | 171 | −19.723 | 8.909 | 4.723 | 1.00 | 0.06 |
| ATOM | 1067 | CG | ASN | 171 | −19.753 | 10.413 | 4.700 | 1.00 | 0.57 |
| ATOM | 1068 | OD1 | ASN | 171 | −19.114 | 11.071 | 5.524 | 1.00 | −0.57 |
| ATOM | 1069 | ND2 | ASN | 171 | −20.561 | 11.010 | 3.787 | 1.00 | −0.80 |
| ATOM | 1070 | N | ASP | 172 | −17.978 | 9.897 | 2.197 | 1.00 | −0.73 |
| ATOM | 1071 | CA | ASP | 172 | −17.227 | 9.998 | 0.930 | 1.00 | 0.36 |
| ATOM | 1072 | C | ASP | 172 | −15.716 | 10.261 | 1.021 | 1.00 | 0.57 |
| ATOM | 1073 | O | ASP | 172 | −14.916 | 9.680 | 0.284 | 1.00 | −0.57 |
| ATOM | 1074 | CB | ASP | 172 | −17.814 | 11.112 | 0.057 | 1.00 | −0.11 |
| ATOM | 1075 | CG | ASP | 172 | −19.152 | 10.542 | −0.367 | 1.00 | 0.91 |
| ATOM | 1076 | OD1 | ASP | 172 | −19.209 | 10.139 | −1.560 | 1.00 | −0.90 |
| ATOM | 1077 | OD2 | ASP | 172 | −20.008 | 10.489 | 0.573 | 1.00 | −0.90 |
| ATOM | 1078 | N | GLU | 173 | −15.304 | 11.299 | 1.818 | 1.00 | −0.73 |
| ATOM | 1079 | CA | GLU | 173 | −13.973 | 11.902 | 1.689 | 1.00 | 0.36 |
| ATOM | 1080 | C | GLU | 173 | −12.855 | 11.173 | 2.482 | 1.00 | 0.57 |
| ATOM | 1081 | O | GLU | 173 | −11.811 | 11.721 | 2.833 | 1.00 | −0.57 |
| ATOM | 1082 | CB | GLU | 173 | −14.018 | 13.421 | 1.961 | 1.00 | 0.00 |
| ATOM | 1083 | CG | GLU | 173 | −13.990 | 13.874 | 3.424 | 1.00 | −0.11 |
| ATOM | 1084 | CD | GLU | 173 | −14.927 | 13.179 | 4.378 | 1.00 | 0.91 |
| ATOM | 1085 | OB1 | GLU | 173 | −15.994 | 12.675 | 3.927 | 1.00 | −0.90 |
| ATOM | 1086 | OE2 | GLU | 173 | −14.529 | 13.053 | 5.576 | 1.00 | −0.90 |
| ATOM | 1087 | N | LYS | 174 | −13.077 | 9.832 | 2.688 | 1.00 | −0.73 |
| ATOM | 1088 | CA | LYS | 174 | −12.115 | 8.932 | 3.302 | 1.00 | 0.36 |
| ATOM | 1089 | C | LYS | 174 | −11.877 | 7.776 | 2.316 | 1.00 | 0.57 |
| ATOM | 1090 | O | LYS | 174 | −12.727 | 7.422 | 1.503 | 1.00 | −0.57 |
| ATOM | 1091 | CB | LYS | 174 | −12.524 | 8.415 | 4.696 | 1.00 | 0.00 |
| ATOM | 1092 | CG | LYS | 174 | −14.025 | 8.393 | 5.033 | 1.00 | 0.00 |
| ATOM | 1093 | CD | LYS | 174 | −14.559 | 9.778 | 5.412 | 1.00 | 0.00 |
| ATOM | 1094 | CE | LYS | 174 | −16.019 | 9.767 | 5:830 | 1.00 | 0.50 |
| ATOM | 1095 | NZ | LYS | 174 | −16.538 | 11.139 | 5.826 | 1.00 | −0.85 |
| ATOM | 1096 | N | PHE | 175 | −10.650 | 7.168 | 2.425 | 1.00 | −0.73 |
| ATOM | 1097 | CA | PHE | 175 | −10.249 | 6.072 | 1.524 | 1.00 | 0.36 |
| ATOM | 1098 | C | PHE | 175 | −10.647 | 4.748 | 2.190 | 1.00 | 0.57 |
| ATOM | 1099 | O | PHE | 175 | −9.828 | 3.923 | 2.572 | 1.00 | −0.57 |
| ATOM | 1100 | CB | PHE | 175 | −8.744 | 6.100 | 1.235 | 1.00 | 0.14 |
| ATOM | 1101 | CG | PHE | 175 | −8.297 | 7.439 | 0.695 | 1.00 | −0.14 |
| ATOM | 1102 | CD1 | PHE | 175 | −8.509 | 7.776 | −0.646 | 1.00 | −0.15 |
| ATOM | 1103 | CD2 | PHE | 175 | −7.736 | 8.389 | 1.561 | 1.00 | −0.15 |
| ATOM | 1104 | CE1 | PHE | 175 | −8.174 | 9.049 | −1.104 | 1.00 | −0.15 |
| ATOM | 1105 | CE2 | PHE | 175 | −7.394 | 9.656 | 1.099 | 1.00 | −0.15 |
| ATOM | 1106 | CZ | PHE | 175 | −7.612 | 9.985 | −0.235 | 1.00 | −0.15 |
| ATOM | 1107 | N | TYR | 176 | −12.011 | 4.643 | 2.374 | 1.00 | −0.73 |
| ATOM | 1108 | CA | TYR | 176 | −12.637 | 3.503 | 3.029 | 1.00 | 0.36 |
| ATOM | 1109 | C | TYR | 176 | −13.936 | 3.142 | 2.274 | 1.00 | 0.57 |
| ATOM | 1110 | O | TYR | 176 | −14.875 | 2.580 | 2.830 | 1.00 | −0.57 |
| ATOM | 1111 | CB | TYR | 176 | −13.012 | 3.806 | 4.499 | 1.00 | 0.14 |
| ATOM | 1112 | CG | TYR | 176 | −11.939 | 4.134 | 5.515 | 1.00 | −0.14 |
| ATOM | 1113 | CD1 | TYR | 176 | −10.656 | 3.580 | 5.472 | 1.00 | −0.15 |
| ATOM | 1114 | CD2 | TYR | 176 | −12.274 | 4.942 | 6.612 | 1.00 | −0.15 |
| ATOM | 1115 | GE1 | TYR | 176 | −9.703 | 3.893 | 6.446 | 1.00 | −0.15 |
| ATOM | 1116 | CE2 | TYR | 176 | −11.338 | 5.214 | 7.610 | 1.00 | −0.15 |
| ATOM | 1117 | CZ | TYR | 176 | −10.059 | 4.689 | 7.523 | 1.00 | 0.08 |
| ATOM | 1118 | OH | TYR | 176 | −9.182 | 4.960 | 8.533 | 1.00 | −0.53 |
| ATOM | 1119 | N | LEU | 177 | −13.977 | 3.405 | 0.928 | 1.00 | −0.73 |
| ATOM | 1120 | GA | LEU | 177 | −15.112 | 2.984 | 0.117 | 1.00 | 0.36 |
| ATOM | 1121 | C | LEU | 177 | −14.725 | 3.058 | −1.365 | 1.00 | 0.57 |
| ATOM | 1122 | O | LEU | 177 | −13.706 | 3.625 | −1.756 | 1.00 | −0.57 |
| ATOM | 1123 | CB | LEU | 177 | −16.402 | 3.789 | 0.387 | 1.00 | 0.00 |
| ATOM | 1124 | CG | LEU | 177 | −16.481 | 5.203 | −0.232 | 1.00 | 0.00 |
| ATOM | 1125 | CD1 | LEU | 177 | −17.870 | 5.799 | 0.006 | 1.00 | 0.00 |
| ATOM | 1126 | CD2 | LEU | 177 | −15.413 | 6.148 | 0.309 | 1.00 | 0.00 |
| ATOM | 1127 | N | SER | 178 | −15.640 | 2.506 | −2.231 | 1.00 | −0.73 |
| ATOM | 1128 | CA | SER | 178 | −15.373 | 2.410 | −3.673 | 1.00 | 0.36 |
| ATOM | 1129 | C | SER | 178 | −15.571 | 3.780 | −4.369 | 1.00 | 0.57 |
| ATOM | 1130 | O | SER | 178 | −16.416 | 3.979 | −5.242 | 1.00 | −0.57 |
| ATOM | 1131 | CB | SER | 178 | −16.316 | 1.376 | −4.309 | 1.00 | 0.28 |
| ATOM | 1132 | OG | SER | 178 | −16.177 | 0.111 | −3.648 | 1.00 | −0.68 |
| ATOM | 1133 | N | ASN | 179 | −14.703 | 4.766 | −3.970 | 1.00 | −0.73 |
| ATOM | 1134 | CA | ASN | 179 | −14.758 | 6.154 | −4.442 | 1.00 | 0.36 |
| ATOM | 1135 | C | ASN | 179 | −13.323 | 6.723 | −4.336 | 1.00 | 0.57 |
| ATOM | 1136 | O | ASN | 179 | −12.447 | 6.207 | −3.643 | 1.00 | −0.57 |
| ATOM | 1137 | CB | ASN | 179 | −15.745 | 6.972 | −3.603 | 1.00 | 0.06 |
| ATOM | 1138 | CG | ASN | 179 | −16.098 | 8.308 | −4.223 | 1.00 | 0.57 |
| ATOM | 1139 | OD1 | ASN | 179 | −15.567 | 8.758 | −5.237 | 1.00 | −0.57 |
| ATOM | 1140 | ND2 | ASN | 179 | −17.038 | 9.022 | −3.546 | 1.00 | −0.80 |
| ATOM | 1141 | N | GLY | 180 | −13.075 | 7.860 | −5.078 | 1.00 | −0.73 |
| ATOM | 1142 | CA | GLY | 180 | −11.823 | 8.563 | 4.894 | 1.00 | 0.36 |
| ATOM | 1143 | C | GLY | 180 | −11.610 | 9.626 | −5.970 | 1.00 | 0.57 |
| ATOM | 1144 | O | GLY | 180 | −10.562 | 9.764 | −6.607 | 1.00 | −0.57 |
| ATOM | 1145 | N | ARG | 181 | −12.650 | 10.522 | −6.105 | 1.00 | −0.73 |
| ATOM | 1146 | CA | ARG | 181 | −12.522 | 11.723 | −6.935 | 1.00 | 0.36 |
| ATOM | 1147 | C | ARG | 181 | −11.928 | 12.880 | −6.088 | 1.00 | 0.57 |
| ATOM | 1148 | O | ARG | 181 | −12.347 | 14.034 | −6.119 | 1.00 | −0.57 |
| ATOM | 1149 | CB | ARG | 181 | −13.862 | 12.123 | −7.557 | 1.00 | 0.00 |
| ATOM | 1150 | CG | ARG | 181 | −14.565 | 10.953 | −8.255 | 1.00 | 0.00 |
| ATOM | 1151 | CD | ARG | 181 | −15.596 | 11.438 | −9.269 | 1.00 | 0.33 |
| ATOM | 1152 | NE | ARG | 181 | −14.923 | 11.965 | −10.454 | 1.00 | −0.84 |
| ATOM | 1153 | CZ | ARG | 181 | −15.458 | 12.630 | −11.472 | 1.00 | 1.20 |
| ATOM | 1154 | NH1 | ARG | 181 | −16.761 | 12.909 | −11.530 | 1.00 | −0.97 |
| ATOM | 1155 | NH2 | ARG | 181 | −14.664 | 13.021 | −12.465 | 1.00 | −0.97 |
| ATOM | 1156 | N | ILE | 182 | −10.817 | 12.525 | −5.349 | 1.00 | −0.73 |
| ATOM | 1157 | CA | ILE | 182 | −10.180 | 13.455 | −4.427 | 1.00 | 0.36 |
| ATOM | 1158 | C | ILE | 182 | −9.191 | 14.368 | −5.196 | 1.00 | 0.57 |
| ATOM | 1159 | O | ILE | 182 | −8.569 | 14.006 | −6.197 | 1.00 | −0.57 |
| ATOM | 1160 | CB | ILE | 182 | −9.496 | 12.654 | −3.286 | 1.00 | 0.00 |
| ATOM | 1161 | CG1 | ILE | 182 | −9.494 | 13.405 | −1.940 | 1.00 | 0.00 |
| ATOM | 1162 | CG2 | ILE | 182 | −8.073 | 12.211 | −3.654 | 1.00 | 0.00 |
| ATOM | 1163 | CD1 | ILE | 182 | −10.872 | 13.479 | −1.293 | 1.00 | 0.00 |
| ATOM | 1164 | N | GLN | 183 | −8.967 | 15.604 | 4.612 | 1.00 | −0.73 |
| ATOM | 1165 | CA | GLN | 183 | −8.019 | 16.538 | −5.218 | 1.00 | 0.36 |
| ATOM | 1166 | C | GLN | 183 | −6.582 | 16.113 | −4.837 | 1.00 | 0.57 |
| ATOM | 1167 | O | GLN | 183 | −5.657 | 16.142 | −5.654 | 1.00 | −0.57 |
| ATOM | 1168 | CB | GLN | 183 | −8.291 | 17.986 | −4.788 | 1.00 | 0.00 |
| ATOM | 1169 | CG | GLN | 183 | −7.346 | 19.002 | −5.441 | 1.00 | 0.06 |
| ATOM | 1170 | CD | GLN | 183 | −7.434 | 18.972 | −6.953 | 1.00 | 0.57 |
| ATOM | 1171 | OE1 | GLN | 183 | −6.677 | 18.307 | −7.659 | 1.00 | −0.57 |
| ATOM | 1172 | NE2 | GLN | 183 | −8.480 | 19.656 | −7.487 | 1.00 | −0.80 |
| ATOM | 1173 | N | ALA | 184 | −6.386 | 15.836 | −3.509 | 1.00 | −0.73 |
| ATOM | 1174 | CA | ALA | 184 | −5.100 | 15.393 | −2.977 | 1.00 | 0.36 |
| ATOM | 1175 | C | ALA | 184 | −5.383 | 14.480 | −1.772 | 1.00 | 0.57 |
| ATOM | 1176 | O | ALA | 184 | −6.519 | 14.344 | −1.316 | 1.00 | −0.57 |
| ATOM | 1177 | CD | ALA | 184 | −4.244 | 16.589 | −2.590 | 1.00 | 0.00 |
| ATOM | 1178 | N | VAL | 185 | −4.295 | 13.807 | −1.267 | 1.00 | −0.73 |
| ATOM | 1179 | CA | VAL | 185 | −4.445 | 12.956 | −0.087 | 1.00 | 0.36 |
| ATOM | 1180 | C | VAL | 185 | −4.353 | 13.875 | 1.149 | 1.00 | 0.57 |
| ATOM | 1181 | O | VAL | 185 | −3.504 | 14.758 | 1.258 | 1.00 | −0.57 |
| ATOM | 1182 | CB | VAL | 185 | −3.335 | 11.882 | −0.019 | 1.00 | 0.00 |
| ATOM | 1183 | CG1 | VAL | 185 | −3.497 | 10.956 | 1.192 | 1.00 | 0.00 |
| ATOM | 1184 | CG2 | VAL | 185 | −3.306 | 11.030 | −1.291 | 1.00 | 0.00 |
| ATOM | 1185 | N | ARG | 186 | −5.251 | 13.576 | 2.142 | 1.00 | −0.73 |
| ATOM | 1186 | CA | ARG | 186 | −5.152 | 14.135 | 3.475 | 1.00 | 0.36 |
| ATOM | 1187 | C | ARG | 186 | −5.910 | 13.161 | 4.391 | 1.00 | 0.57 |
| ATOM | 1188 | O | ARG | 186 | −6.786 | 12.404 | 3.975 | 1.00 | −0.57 |
| ATOM | 1189 | CB | ARG | 186 | −5.793 | 15.529 | 3.590 | 1.00 | 0.00 |
| ATOM | 1190 | CG | ARG | 186 | −5.076 | 16.403 | 4.631 | 1.00 | 0.00 |
| ATOM | 1191 | CD | ARG | 186 | −5.996 | 17.424 | 5.300 | 1.00 | 0.33 |
| ATOM | 1192 | NE | ARG | 186 | −6.855 | 16.787 | 6.309 | 1.00 | −0.84 |
| ATOM | 1193 | CZ | ARG | 186 | −7.425 | 17.400 | 7.357 | 1.00 | 1.20 |
| ATOM | 1194 | NH1 | ARG | 186 | −7.338 | 18.717 | 7.545 | 1.00 | −0.97 |
| ATOM | 1195 | NH2 | ARG | 186 | −8.096 | 16.671 | 8.248 | 1.00 | −0.97 |
| ATOM | 1196 | N | CYS | 187 | −5.610 | 13.275 | 5.733 | 1.00 | −0.73 |
| ATOM | 1197 | CA | CYS | 187 | −6.373 | 12.467 | 6.682 | 1.00 | 0.36 |
| ATOM | 1198 | C | CYS | 187 | −7.739 | 13.172 | 6.785 | 1.00 | 0.57 |
| ATOM | 1199 | O | CYS | 187 | −7.830 | 14.376 | 7.059 | 1.00 | −0.57 |
| ATOM | 1200 | CE | CYS | 187 | −5.761 | 12.453 | 8.087 | 1.00 | 0.23 |
| ATOM | 1201 | SG | CYS | 187 | −4.017 | 11.935 | 8.077 | 1.00 | −0.41 |
| ATOM | 1202 | N | SER | 188 | −8.848 | 12.397 | 6.555 | 1.00 | −0.73 |
| ATOM | 1203 | CA | SER | 188 | −10.179 | 12.898 | 6.894 | 1.00 | 0.36 |
| ATOM | 1204 | C | SER | 188 | −10.282 | 12.926 | 8.438 | 1.00 | 0.57 |
| ATOM | 1205 | O | SER | 188 | −9.469 | 12.374 | 9.180 | 1.00 | −0.57 |
| ATOM | 1206 | CB | SER | 188 | −11.312 | 12.034 | 6.317 | 1.00 | 0.28 |
| ATOM | 1207 | OG | SER | 188 | −12.557 | 12.336 | 6.950 | 1.00 | −0.68 |
| ATOM | 1208 | N | ALA | 189 | −11.403 | 13.580 | 8.918 | 1.00 | −0.73 |
| ATOM | 1209 | CA | ALA | 189 | −11.798 | 13.392 | 10.311 | 1.00 | 0.36 |
| ATOM | 1210 | C | ALA | 189 | −12.176 | 11.921 | 10.572 | 1.00 | 0.57 |
| ATOM | 1211 | O | ALA | 189 | −12.065 | 11.403 | 11.679 | 1.00 | −0.57 |
| ATOM | 1212 | CB | ALA | 189 | −12.983 | 14.283 | 10.648 | 1.00 | 0.00 |
| ATOM | 1213 | N | GLY | 190 | −12.679 | 11.228 | 9.496 | 1.00 | −0.73 |
| ATOM | 1214 | CA | GLY | 190 | −13.096 | 9.846 | 9.619 | 1.00 | 0.36 |
| ATOM | 1215 | C | GLY | 190 | −11.948 | 8.831 | 9.659 | 1.00 | 0.57 |
| ATOM | 1216 | O | GLY | 190 | −12.181 | 7.638 | 9.822 | 1.00 | −0.57 |
| ATOM | 1217 | N | ILE | 191 | −10.675 | 9.328 | 9.444 | 1.00 | −0.73 |
| ATOM | 1218 | CA | ILE | 191 | −9.491 | 8.475 | 9.614 | 1.00 | 0.36 |
| ATOM | 1219 | C | ILE | 191 | −8.973 | 8.525 | 11.078 | 1.00 | 0.57 |
| ATOM | 1220 | O | ILE | 191 | −8.190 | 7.676 | 11.515 | 1.00 | −0.57 |
| ATOM | 1221 | CB | ILE | 191 | −8.410 | 8.811 | 8.542 | 1.00 | 0.00 |
| ATOM | 1222 | CG1 | ILE | 191 | −8.950 | 8.417 | 7.142 | 1.00 | 0.00 |
| ATOM | 1223 | CG2 | ILE | 191 | −7.063 | 8.133 | 8.824 | 1.00 | 0.00 |
| ATOM | 1224 | CD1 | ILE | 191 | −7.955 | 8.493 | 5.993 | 1.00 | 0.00 |
| ATOM | 1225 | N | THR | 192 | −9.358 | 9.604 | 11.863 | 1.00 | −0.73 |
| ATOM | 1226 | CA | THR | 192 | −9.242 | 9.422 | 13.317 | 1.00 | 0.36 |
| ATOM | 1227 | C | THR | 192 | −10.476 | 8.605 | 13.757 | 1.00 | 0.57 |
| ATOM | 1228 | O | THR | 192 | −11.364 | 8.262 | 12.974 | 1.00 | −0.57 |
| ATOM | 1229 | CE | THR | 192 | −9.034 | 10.707 | 14.138 | 1.00 | 0.28 |
| ATOM | 1230 | OG1 | THR | 192 | −8.730 | 10.340 | 15.495 | 1.00 | −0.6S |
| ATOM | 1231 | CG2 | THR | 192 | −10.204 | 11.677 | 14.147 | 1.00 | 0.00 |
| ATOM | 1232 | N | GLY | 193 | −10.467 | 8.169 | 15.058 | 1.00 | −0.73 |
| ATOM | 1233 | CA | GLY | 193 | −11.538 | 7.332 | 15.566 | 1.00 | 0.36 |
| ATOM | 1234 | C | GLY | 193 | −11.291 | 5.874 | 15.171 | 1.00 | 0.57 |
| ATOM | 1235 | O | GLY | 193 | −11.033 | 5.005 | 16.005 | 1.00 | −0.57 |
| ATOM | 1236 | N | THR | 194 | −11.287 | 5.603 | 13.822 | 1.00 | −0.73 |
| ATOM | 1237 | CA | THR | 194 | −10.957 | 4.267 | 13.316 | 1.00 | 0.36 |
| ATOM | 1238 | C | THR | 194 | −9.541 | 3.884 | 13.791 | 1.00 | 0.57 |
| ATOM | 1239 | O | THR | 194 | −9.232 | 2.733 | 14.098 | 1.00 | −0.57 |
| ATOM | 1240 | CB | THR | 194 | −10.990 | 4.181 | 11.781 | 1.00 | 0.28 |
| ATOM | 1241 | OG1 | THR | 194 | −10.125 | 5.184 | 11.237 | 1.00 | −0.68 |
| ATOM | 1242 | CG2 | THR | 194 | −12.395 | 4.367 | 11.227 | 1.00 | 0.00 |
| ATOM | 1243 | N | ASN | 195 | −8.628 | 4.925 | 13.833 | 1.00 | −0.73 |
| ATOM | 1244 | CA | ASN | 195 | −7.247 | 4.667 | 14.237 | 1.00 | 0.36 |
| ATOM | 1245 | C | ASN | 195 | −7.150 | 4.286 | 15.725 | 1.00 | 0.57 |
| ATOM | 1246 | O | ASN | 195 | −6.147 | 3.731 | 16.163 | 1.00 | −0.57 |
| ATOM | 1247 | CB | ASN | 195 | −6.322 | 5.859 | 14.001 | 1.00 | 0.06 |
| ATOM | 1248 | CG | ASN | 195 | −5.518 | 5.616 | 12.751 | 1.00 | 0.57 |
| ATOM | 1249 | OD1 | ASN | 195 | −4.437 | 5.037 | 12.742 | 1.00 | −0.57 |
| ATOM | 1250 | ND2 | ASN | 195 | −6.116 | 6.000 | 11.593 | 1.00 | −0.80 |
| ATOM | 1251 | N | VAL | 196 | −8.173 | 4.675 | 16.553 | 1.00 | −0.73 |
| ATOM | 1252 | GA | VAL | 196 | −8.186 | 4.268 | 17.970 | 1.00 | 0.36 |
| ATOM | 1253 | C | VAL | 196 | −8.684 | 2.801 | 18.100 | 1.00 | 0.57 |
| ATOM | 1254 | O | VAL | 196 | −8.458 | 2.112 | 19.091 | 1.00 | −0.57 |
| ATOM | 1255 | CB | VAL | 196 | −9.041 | 5.252 | 18.803 | 1.00 | 0.00 |
| ATOM | 1256 | CG1 | VAL | 196 | −9.143 | 4.835 | 20.274 | 1.00 | 0.00 |
| ATOM | 1257 | CG2 | VAL | 196 | −8.461 | 6.672 | 18.734 | 1.00 | 0.00 |
| ATOM | 1258 | N | VAL | 197 | −9.499 | 2.343 | 17.084 | 1.00 | −0.73 |
| ATOM | 1259 | GA | VAL | 197 | −10.008 | 0.970 | 17.082 | 1.00 | 0.36 |
| ATOM | 1260 | C | VAL | 197 | −8.930 | −0.001 | 16.528 | 1.00 | 0.57 |
| ATOM | 1261 | O | VAL | 197 | −8.840 | −1.165 | 16.921 | 1.00 | −0.57 |
| ATOM | 1262 | CB | VAL | 197 | −11.318 | 0.865 | 16.258 | 1.00 | 0.00 |
| ATOM | 1263 | CG1 | VAL | 197 | −11.849 | −0.572 | 16.203 | 1.00 | 0.00 |
| ATOM | 1264 | CG2 | VAL | 197 | −12.416 | 1.764 | 16.840 | 1.00 | 0.00 |
| ATOM | 1265 | N | LYS | 198 | −8.224 | 0.443 | 15.430 | 1.00 | −0.73 |
| ATOM | 1266 | CA | LYS | 198 | −7.302 | −0.427 | 14.695 | 1.0 | 0.36 |
| ATOM | 1267 | C | LYS | 198 | −5.809 | −0.215 | 15.050 | 1.00 | 0.57 |
| ATOM | 1268 | O | LYS | 198 | −4.925 | −0.968 | 14.633 | 1.00 | −0.57 |
| ATOM | 1269 | CB | LYS | 198 | −7.475 | −0.232 | 13.181 | 1.00 | 0.00 |
| ATOM | 1270 | CG | LYS | 198 | −8.860 | −0.606 | 12.633 | 1.00 | 0.00 |
| ATOM | 1271 | CD | LYS | 198 | −9.229 | −2.071 | 12.890 | 1.00 | 0.00 |
| ATOM | 1272 | CE | LYS | 198 | −10.364 | −2.590 | 12.018 | 1.00 | 0.50 |
| ATOM | 1273 | NZ | LYS | 198 | −11.579 | −1.805 | 12.213 | 1.00 | −0.85 |
| ATOM | 1274 | N | LYS | 199 | −5.522 | 0.937 | 15.735 | 1.00 | −0.73 |
| ATOM | 1275 | CA | LYS | 199 | −4.171 | 1.339 | 16.142 | 1.00 | 0.36 |
| ATOM | 1276 | G | LYS | 199 | −4.274 | 1.966 | 17.558 | 1.00 | 0.57 |
| ATOM | 1277 | O | LYS | 199 | −5.291 | 1.874 | 18.244 | 1.00 | −0.57 |
| ATOM | 1278 | CB | LYS | 199 | −3.569 | 2.316 | 15.113 | 1.00 | 0.00 |
| ATOM | 1279 | CG | LYS | 199 | −3.328 | 1.723 | 13.724 | 1.00 | 0.00 |
| ATOM | 1280 | CD | LYS | 199 | −2.143 | 0.762 | 13.716 | 1.00 | 0.00 |
| ATOM | 1281 | CE | LYS | 199 | −1.898 | 0.233 | 12.318 | 1.00 | 0.50 |
| ATOM | 1282 | NZ | LYS | 199 | −0.654 | −0.537 | 12.306 | 1.00 | −0.85 |
| ATOM | 1283 | N | CYS | 200 | −3.141 | 2.584 | 18.032 | 1.00 | −0.73 |
| ATOM | 1284 | CA | CYS | 200 | −3.172 | 3.431 | 19.230 | 1.00 | 0.36 |
| ATOM | 1285 | C | CYS | 200 | −2.015 | 4.435 | 19.050 | 1.00 | 0.57 |
| ATOM | 1286 | O | CYS | 200 | −1.040 | 4.187 | 18.337 | 1.00 | −0.57 |
| ATOM | 1287 | CB | CYS | 200 | −3.003 | 2.596 | 20.503 | 1.00 | 0.23 |
| ATOM | 1288 | SG | CYS | 200 | −3.390 | 3.506 | 22.035 | 1.00 | −0.41 |
| ATOM | 1289 | N | GLN | 201 | −2.145 | 5.617 | 19.736 | 1.00 | −0.73 |
| ATOM | 1290 | CA | GLN | 201 | −1.084 | 6.607 | 19.779 | 1.00 | 0.36 |
| ATOM | 1291 | C | GLN | 201 | 0.022 | 6.167 | 20.734 | 1.00 | 0.45 |
| ATOM | 1292 | O | GLN | 201 | −0.160 | 5.523 | 21.764 | 1.00 | −0.57 |
| ATOM | 1293 | CB | GLN | 201 | −1.578 | 8.021 | 20.127 | 1.00 | 0.00 |
| ATOM | 1294 | CG | GLN | 201 | −2.038 | 8.246 | 21.575 | 1.00 | 0.06 |
| ATOM | 1295 | CD | GLN | 201 | −3.349 | 7.574 | 21.916 | 1.00 | 0.57 |
| ATOM | 1296 | OE1 | GLN | 201 | −4.059 | 7.000 | 21.098 | 1.00 | −0.57 |
| ATOM | 1297 | NE2 | GLN | 201 | −3.712 | 7.665 | 23.226 | 1.00 | −0.80 |
| TER | 1298 | | GLN | 201 | | | | | |
| HETATM | 1299 | ZN | ZN | 1 | −5.003 | −11.565 | −3.977 | 1.00 | 2.00 |
| HETATM | 1300 | ZN | ZN | 2 | −11.732 | −1.355 | 0.692 | 1.00 | 2.00 |
| END | | | | | | | | | |

What we claim is:

1. A method for identifying a compound capable of modulating the hydrolase activity of a CLCA protein which method comprises:
(a) subjecting one or more test compounds to a screen comprising at least one protein selected from the group consisting of: a CLCA protein or a fragment thereof; a homologue of a CLCA protein or a fragment thereof; and
(b) measuring the hydrolase activity of the CLCA protein or homologue or fragment; and
(c) comparing the measured hydrolase activity with the hydrolase activity of the CLCA protein or homologue or fragment in the absence of the test compound.

2. A method as claimed in claim 1 wherein at least one of the proteins is selected from the group consisting of: a mammalian CLCA protein or a fragment thereof; a homologue of a mammalian CLCA protein or a fragment thereof.

3. A method as claimed in claim 2 wherein at least one of the proteins is selected from the group consisting of: a human CLCA protein or a fragment thereof; a homologue of a human CLCA protein or a fragment thereof.

4. A method as claimed in claim 3 wherein at least one of the proteins is selected from the group consisting of: hCLCA1 or a fragment thereof; a homologue of hCLCA1 or a fragment thereof.

5. A method as claimed in claim 1 wherein the CLCA protein or fragment thereof or the homologue of a CLCA protein or fragment thereof is present as a fusion protein.

6. A method to design a compound capable of modulating CLCA hydrolase activity which comprises molecular modelling based on the interaction of a potential modulator with a hydrolase domain of a CLCA protein or homologue or fragment of either, wherein the three-dimensional structure of the hydrolase domain is defined by the set of atomic coordinates shown in Table 1.

7. A method to design a compound capable of modulating CLCA hydrolase activity which comprises molecular modelling based on the interaction of a potential modulator with the active site of a hydrolase domain of a CLCA protein or homologue or fragment of either, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1.

8. A method for in silico screening for a compound capable of modulating CLCA hydrolase activity which comprises
a) searching a structural database of compounds; and
b) selecting a compound structure that may interact with a hydrolase domain of a
CLCA protein or homologue or fragment of either, wherein the three-dimensional structure of the hydrolase domain is defined by the set of atomic coordinates shown in Table 1.

9. A method for in silico screening for a compound capable of modulating CLCA hydrolase activity which comprises
a) searching a structural database of compounds; and
b) selecting a compound structure that may interact with the active site of a hydrolase domain of a CLCA protein or homologue or fragment of either, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1.

10. A method for designing an antibody capable of modulating the hydrolase activity of a CLCA protein which method comprises using the three-dimensional structure of a CLCA hydrolase domain to identify suitable epitopes in the vicinity of the active site, wherein the three-dimensional structure of the hydrolase domain is defined by the set of atomic coordinates shown in Table 1 and the active site comprises the amino acid residues within 15 Å of atom Zn-1300 in the set of atomic coordinates shown in Table 1.

11. A method as claimed in claim 10 wherein the epitopes include only surface residues within 15 Å of atom Zn-1300 in the set of atomic coordinates shown in Table 1.


SEQUENCE LISTING (44 sequences)

SEQ ID NO: 1 - protein, 302 aa, Bos taurus
ILFLTLHLLPGMKSSMVNLINNGYDGIVIAINPSVPEDEKLIENIKEMVTEASTYLFHATKRRVYFRNVSILIPMTWKSKSEYFIPKQESYDQADVIVANPYLKYGDDPYTLQYGRCGEKGKYIHFTPNFLLTNNFHIYGSRGRVFVHEWAHLRWGIFDEYNVDQPFYISRKNTIEATRCSTHITGINVVFKKCPGGSCITSLCRRDSQTGLYEAKCTFLPKKSQTAKESIMFMPSLHSVTEFCTEKTHNTEAPNLQNKMCNGKSTWDVIMNSVDFQNTSPMTEMNPPTHPTFSLLKSKQRV

SEQ ID NO: 2 - protein, 306 aa, Homo sapiens
MGPFKSSVFILILHLLEGALSNSLIQLNNNGYEGIVVAIDPNVPEDETLIQQIKDMVTQASLYLFEATGKRFYFKNVAILIPETWKTKADYVRPKLETYKNADVLVAESTPPGNDEPYTEQMGNCGEKGERIHLTPDFIAGKKLAEYGPQGKAFVHEWAHLRWGVFDEYNNDEKFYLSNGRIQAVRCSAGITGTNVVKKCQGGSCYTKRCTFNKVTGLYEKGCEFVLQSRQTEKASIMFAQHVDSIVEFCTEQNHNKEAPNKQNQKCNLRSTWEVIRDSEDFKKTTPMTTQPPNPTFSLLQIGQRI

SEQ ID NO: 3 - protein, 306 aa, Homo sapiens
MGLFRGFVFLLVLCLLHQSNTSFIKLNNNGFEDIVIVIDPSVPEDEKIIEQIEDMVTTASTYLFEATEKRFFFKNVSILIPENWKENPQYKRPKHENHKHADVIVAPPTLPGRDEPYTKQFTECGEKGEYIHFTPDLLLGKKQNEYGPPGKLFVHEWAHLRWGVFDEYNEDQPFYRAKSKKIEATRCSAGISGRNRVYKCQGGSCLSRACRIDSTTKLYGKDCQFFPDKVQTEKASIMFMQSIDSVVEFCNEKTHNQEAPSLQNIKCNFRSTWEVISNSEDFKNTIPMVTPPPPPVFSLLKIRQRI

SEQ ID NO: 4 - protein, 304 aa, Homo sapiens
GPICNLKFVTLLVALSSELPFLGAGVQLQDNGYNGLLIAINPQVPENQNLISNIKEMITEASFYLFNATKRRVFFRNIKILIPATWKANNNSKIKQESYEKANVIVTDWYGAHGDDPYTLQYRGCGKEGKYIHFTPNFLLNDNLTAGYGSRGRVFVHEWAHLRWGVFDEYINDKPFYINGQNQIKVTRCSSDITGIFVCEKGPCPQENCIISKLFKEGCTFIYNSTQNATASIMFMQSLSSVVEFCNASTHNQEAPNLQNQMCSLRSAWDVITDSADFHHSFPMNGTELPPPPTFSLVQAGDKV

SEQ ID NO: 5 - protein, 259 aa, Homo sapiens
FSLKVILFLSLLLSPVLKSSLVTLNNNGYDGIVIAINPSVPEDEKLIQNIKEMVTEASTHLFHATKQRAYFRNVSILIPMTYKSKSEYLIPKQETYDQADVIVADLYLKYGDDPYTLQYGQCGDKGQYIHFTPNFLLTNNLATYGPRGKVFVHGWAHLRWGVFDEYNVDQPFYISRRNTTEATRCSTRITVYMVLNECKGASCIARPFRRDSQTGLYEAKCTFIPKRSQTAKESIVFMQNLDSVTEFCTEKTHNKEAPN

SEQ ID NO: 6 - protein, 279 aa, Mus musculus
LKLKENGYDGLLVAINPRVPEDLKLITNIKEMITEASFYLFNATKRRVFFRNVQILVPATWTDHNYSRVRQESYDKANVIVAEQSEEHGDDPYTLQHRGCGQEGRYIHFTPSFLLNDELAAGYGARGRVFVHEWAHLRWGVFDEYNNDKPFYVNGRNEIQVTRCSSDITGVFVCEKGLCPHEDCIISKIFREGCTFLYNSTQNATGSIMFMPSLPSVVEFCNESTHNQEAPNLQNQVCSLRSTWDVITASSDLNHSLPVHGVGLPAPPTFSLLQAGDRV

SEQ ID NO: 7 - protein, 308 aa, Mus musculus
MVPGLQVLLFLTLHLLQNTESSMVHLNSNGYEGVVIAINPSVPEDERLIPSIKEMVTQASTYLFEASQGRVYFRNISILVPMTWKSKPEYLMPKRESYDKADVIVADPHLQHGDDPYTLQYGQCGDRGQYIHFTPNFLLTDNLRIYGPRGRVFVHEWAHLRWGVFDEYNVDQPFYMSRKNTIEATRCSTRITGTNVVHNCERGNCVTRACRRDSKTRLYEPKCTFIPDKIQTAGASIMFMQNLNSVVEFCTEKNHNAEAPNLQNKMCNRRSTWDVIKTSADFQNAPPMRGTEAPPPPTFSLLKSRRRV

SEQ ID NO: 8 - protein, 308 aa, Mus musculus
MVPGLQVLLFLTLHLLQNTESSMVHLNSNGYEGVVIAINPSVPEDERLIPSIKEMVTQASTYLFEASQGRVYFRNISILVPMTWKSKSEYLMPKRESYDKADVIVADPHLQHGDDPYTLQYGQCGDRGQYIHFTPNFLLTDNLRIYGPRGRVFVHEWAHLRWGVFDEYNVDRPFYISRKNTIEATRCSASITGKKVVHECQRGSCVTRACRRDSKTRLYEPKCTFIPDKIQTAGASIMFMQNLNSVVEFCTENNHNAEAPNLQNKMCNRRSTWDVIKASADFQNSPPMRGTEAPPPPTFSLLKSRRRV

SEQ ID NO: 9 - protein, 307 aa, Mus musculus
MESLKSPVFLLILHLLEGVLSESLIQLNNNGYEGIVIAIDHDVPEDEALIQHIKDMVTQASPYLFEATGKRFYFKNVAILIPESWKAKPEYTRPKLETFKNADVLVSTTSPLGNDEPYTEHIGACGEKGIRIHLTPDFLAGKKLTQYGPQDRTFVHEWAHFRWGVFNEYNNDEKFYLSKGKPQAVRCSAAITGKNQVRRCQGGSCITNGKCVIDRVTGLYKDNCVFVPDPHQNEKASIMFNQNINSVVEFCTEKNHNQEAPNDQNQRCNLRSTWEVIQESEDFKQTTPMTAQPPAPTFSLLQIGQRI

SEQ ID NO: 10 - protein, 308 aa, Mus musculus
MVPGLQVLLFLTLHLLQNTESSMVHLNSNGYEGVVIAINPSVPEDERLIPSIKEMVTQASTYLFEATERRFYFRNVSILVPITWKSKTEYLTPKQESYDQADVIVADPHLQHGDDPYTLQYGQCGDRGQYIHFTPNFLLTDNLGIYGPRGRVFVHEWAHLRWGVFDEYNMDRPFYMSRKNTVEATRCSTDITGTSVVRECQGGSCVSRRCRRDAKTGMQEAKCTFIPNKSQTARGSIMFMQSLDSVVEFCTEKTHNVEAPNLQNKMCNLRSTWDVIKASADFQNASPMTGTEAPPLPTFSLLKSRQRV

SEQ ID NO: 11 - protein, 306 aa, Sus scrofa
MGSFRSSLFILVLHLLEGAQSNSLIQLNGNGYEGIVIAIDPNVPEDERLIQNIKDMVTKASPYLFEATEKRFYFKNVAILIPASWKAKPEYVKPKLETYKNADVVVTEPNPPENDGPYTEQMGNCGEKGEKIYFTPDFVAGKKVLQYGPQGRVFVHEWAHLRWGVFNEYNNEQKFYLSNKKEQPVICSAAIRGTNVLPQCQGGSCVTKPCRADRVTGLFQKECEFIPDPQQSEKASIMFAQSIDTVVEFCKEKNHNKEAPNDQNQKCNLRSTWEVIQDSEDFKKTTPMTTQPPAPTFSLLQIGQRI

SEQ ID NO: 12 - protein, 308 aa, Bos taurus
MVPRLTVILFLTLHLLPGMKSSMVNLINNGYDGIVIAINPSVPEDEKLIQNIKEMVTEASTYLFHATKRRVYFRNVSILIPMTWKSKSEYLMPKQESYDQAEVIVANPYLKHGDDPYTLQYGRCGEKGQYIHFTPNFLLTNNLPIYGSRGRAFVHEWAHLRWGIFDEYNGDQPFYISRRNTIEATRCSTHITGTNVIVKCQGGSCITRPCRRDSQTGLYEAKCTFIPEKSQTARESIMFMQSLHSVTEFCTEKTHNVEAPNLQNKMCNGKSTWDVIMNSTDFQNTSPMTEMNPPTQPTFSLLKSKQRV

SEQ ID NO: 13 - protein, 247 aa, Ciona intestinalis
ESTTLLNSIKAAWTEASAALYTATRKRAYFGNITILVPKSWNGTYKRAFDETYDAADVVVTNTNRVRGNIPYVLQPGGCGEPGTRIFTTRDYYTNDTYVESFGQRGKVMVHEWSHYRWGVFDEIASGDYAPFYISSTGTIEATRCSLGIQGENMIVQNNEIVQDVCNYDPQTLLPNSTDCKFILAWDQDLDLKASIMSYQYVNEINGFCDDNDNDPLNRHNREAPNEHNDKCNKRSVWDVITSSVDF

SEQ ID NO: 14 - protein, 274 aa, Ciona intestinalis
  MISC_FEATURE 49: any natural amino acid residue
NPAVPEDPNLVSAIQSSWIEASGDLYTATRQRSYFGEITILIPKTWSKXKLVINGSESYETADVLIAEANPVYQDTPYTLQYGNCGETASYIHLTPDYLTNQSFVEDFGPRGKAIVHEWAHLRWGVFDETYTTGYSPYYYDSHGTVQATRCPSTLDGKNKVVDYSTGNSRDCQRNLENGLMEDGCLFLPYAEQSADLTTSLMSHQYLSQVTMFCHNDETDSYNHHNREAPNEQNRLCDLKSAWEVIMESKDFLNNANPRNMVXNTNPIFRLVQI

SEQ ID NO: 15 - protein, 282 aa, Ciona intestinalis
VTLVNNGYDGIVVAINPAVAEDETLINKIRNMFTRASPTLFTATKKRAYFRNINILVPKTWTSGSYQTAVGLTYRKADVIIAPPNPVRGDNPYVLQTGACGEPGTHMHLTPEWVNDTRESVYGPSDKAIVHEWAHLRWGVFDEYATGDHKRHYLDSNNVLQGTRCPLSIRGVNREYVPPYQVLNQTCIINQTTLLPASDTCHFIPGIEQPRGLKTSMMFYSYLSSVIEFCHSDPSDPVNQHNTEADNEQNAKCNLRSTWDVITSTSDFSGGSNPPNPTLTNL

SEQ ID NO: 16 - protein, 286 aa, Ciona intestinalis
SEVNLVNNGYEGIVVAINPSIPEDASLVDNIKTLLNEASPILWSATKNRAYFGEVTILVPSTWTGSYTQATHGQVYNKADIIVADPNPQYMDTPYTIQYQQCGDPGEYIHLTPNFLSQAGYEQNYGNKGKALVHEWAHLRWGVYDEYASEGYAPFYYSNRGGGQPYMEATRCPLALGGVTRYPNPANGNQLEHCTSDPNNNFLPLEGCLFFPFSELGQPDDLSASLLSHQFVDQVVDFCHNDTNDPTNLHNKEAPNEHNRLCDQRSVWEIMMASRDFNAVNHPNPT

SEQ ID NO: 17 - protein, 273 aa, Ciona intestinalis
  MISC_FEATURE 267: any natural amino acid residue
VTLVGNKYKGIVVAINPSIPEDQDLINNIKALLNEASPILWSATKNRAYFGEVTILVPSTWTGSYTQATHGQVYNKADIIVADPNPQYMDTPYTIQYQQCGDPGEYIHLTPNFINEKNDFVENYGSKGKALVHEWAHLRWGIYDEYASEGYDPFYYSSTQYVQPTLEATRCPLSVAGMMLYLDPLSGKFEFCTSNPENNFLPEEGCIFFPRSKEGQPADLIYSFSLTQVVDFCHNDTNDPTNLHNKEAPNEHNRLCDQRSVWEVMNXSSDFKQ

SEQ ID NO: 18 - protein, 279 aa, Ciona intestinalis
VKLQSNGYDGVLVAINPAVPENETLIRNIRASIDLIGATSSHSLFILTKKRAYFRNINILVPKTWTGARYDTAIGLSYRKADVIVAPANSAKGNNPYTRQTGGCGDPGTYIHITPEYVYNPQEHLYGPRGKKAIVHEWSHLRWGVFDEYATGNHKRHYIDSNNILQATRCPLSLRGMNIEYAPPYNTRCAVNRSSLLPLTENCYFFPASRQPRGLNSSMMSFSYLHSVEAFCHNDPNEPINFHNSEADNEQNAKCNLKSLWEVIGASPDFREGANPPNP

SEQ ID NO: 19 - protein, 241 aa, Danio rerio
SVFVVLWMLLPYPFTGIKLDGGGYVDISIAIGAKVKQDDTLIDKIKEMVTDGSFYLYHALDKKVYLKDATILVPSQWSCKSCSIARTELFEKAQIKIDHAKLMEPRTKLYGECGVGGEYIHFTPDFLLNDSAIQMYGPRGKVFLHEWAHLRWGVYDEYNEEKPFYLSNGRVEYTRCTTNIEGQCFEINGGSLQSCRINPETFLPSSDCELSPNKDQNTDSSVMCSPSLQSLTTFCRETEHN

SEQ ID NO: 20 - protein, 268 aa, Gallus gallus
  MISC_FEATURE 39: any natural amino acid residue
MGVFRSLIFLLSFQLLHVAKGSMVKLNESGYEDLVVCNXSQRDRRCQHHPEHKGNDQRCFXLFVXSYKTSIFLQALXRIILPKTWKKNSTYSRLKTESYNKADVIIADPYLKYGDDPYTLQYGGCAMKGRYIHFTPNFLLDSSLIKVYGERGRVLVHEWAHTSVGCVXRIXXRRNLFDVSENARVEPTRCSAGVTWXTCIPKLQWKTVYDKRMPSMMVSYMKLGCGIGNGSSIKKRKNSIMYMQSLPSVVESVIKILINSEVQNMRNR

SEQ ID NO: 21 - protein, 192 aa, Gallus gallus
MGVFRSLIFLLSFQLLHVAKGSMVKLNESGYEGLVVAINPSVTEDANIILNTKAMIKDASNYLFEATKHRFFFKSVKIILPKTWKKNSTYSRLKTESYNKADVIIADPYLKYGDDPYTLQYGGCAMKGRYIHFTPNFLLDSSLIKVYGERGRVFVHEWAHLRWGVFDEYNNDAPFYVSENARVEPTRCSAGV

SEQ ID NO: 22 - protein, 202 aa, Salmo salar
VLLLVYLSGSTFGIKLTGNGYTDILIAINPVVPEDPVLITQIEEMIKEASRHLLNATKKHLYFKEVAILVPPNWNKGNYSKAKTEVYNKANIIIDEPNRLHGDQPYTLQYGECGSEGQYIHLTPDFMLNDDVSKYYGPRGKVFVHEWAHLRWGVFDEYNEEKPFYLSGSIIEATRCTINITGKYIHKRDQKDCTTDPVTGLY

SEQ ID NO: 23 - protein, 202 aa, Strongylocentrotus purpuratus
  MISC_FEATURE 186: any natural amino acid residue
DVPEDQTIIDNLIDIFSSGSGHLFTATRRRAYWRNITILIPKTWTPKPEYEPARTESFETANVIIDTANPEWEDNPYTLQLGGCGVHGEYIHLTPSYITDRANSEYIWGSMGKLLIHEWGHLRWGLFDEYHTDDDGVQKFYADSRGEIVATRCTDQLNGEALNINTFAPCQRDRDTGLYEDDCFYXPDLEGXTSPGSIMYAQ

SEQ ID NO: 24 - protein, 192 aa, Strongylocentrotus purpuratus
GRILMSVVVCCLVLFSGVSGSDLRNSITIQDGGYENVLIAINKDVPEDQTIIDNLIDIFSSGSGHLFTATRRRAYWRNITILIPKTWTPKPEYEPARTESFETANVIIDTANPEWEDNPYTLQLGGCGVHGEYIHLTPSYITDRANSEYIWGSMGKLLIHEWSHLRWGLFDEYHTDDDGVQKFYADSRGVRS

SEQ ID NO: 25 - protein, 131 aa, Strongylocentrotus purpuratus
TILLLEIFLVEVVTGQKNTINLNNGAYSNLLIAIDKNVAEDLNIIDNIKTMFTSSSERLYLASKQHVYWKHIKILVPNTWSIQSGYQFSRTETLESANIILHNFHDDEPFVDNLAGCGKEGTLMHMTPGYI

SEQ ID NO: 26 - protein, 203 aa, Xenopus tropicalis
ASSYLFQATKKRLYIRSAKILIPNTWATNSSYGRPKLESYDKADVIVAPPFVQGDDPYTLQFGGCGEKGKYIHFTPNFLVNDEKMLPIYGPRGRVFVHEWAHFRWGVFDEYNYNRPYYFSENRKVEATRCPLKLKGLNLIDVCQRGVCNLEPCEYDKNTGLYEEDCKFYPDRDILVEESVMYAQMFEPVHAFCDSSSHNSEAP

SEQ ID NO: 27 - protein, 108 aa, Xenopus laevis
DSLVQLKNNGYEDIIIAVNPEVPEDGKIIEQIKKMLTDASSYLFQATKKRIYIRSAKILIPNSWTSNSSYGRPKLESYDKADVIVASPFIHGDDPYTLPVWRLWRKGK

SEQ ID NO: 28 - protein, 124 aa, Xenopus laevis
ATRCPLKMQGSYLIEVCQRGICNLEACEYDENTGLYEEDCKFYPKMDSNVEESVMYAQMMEPVHAFCNSSSHNSEAPNQQNRLCSQQSTWDVISKSSDIQSSPPLMDSNIPAPVVSLLQYKDRV

SEQ ID NO: 29 - protein, 96 aa, Xenopus tropicalis
DSLVQLKNNGYEDIIIAVNPQVPEDGKIIENIKKMLTDASSYLFQATKKRLYIRSAKILIPNTWATNSSYGRPKLESYDKADVIVAPPFVQRDDPY

SEQ ID NO: 30 - protein, 201 aa, Rattus norvegicus
GRDEPYTRQFTKCGKKAEYIHFTPDFVLGRKQKEYGDSGRLLVHEWAHLRWGVFDEYNEDQPFYSASSKKIEATRHVLTPKCSTGIKGMNKAQVCQGGSCITRNCRRNSTTQLYEKDCQFFPDKVQTEKSSIMFMQSIDSVTEFCKKENHNREAPTLHNQKCDYRSTWEVISNSEDFKNSTPMEMPPSPPFFSLLRISERI

SEQ ID NO: 31 - protein, 333 aa, Rattus norvegicus
VKSSKVHLNNNGYEGVVIAINPSVPEDERLIPSLKAKCLGRSGVLSGAENHELSSRALCCWGCFGFLAVPHNAAYTADHKGNQADVIVADPHLKYGDDPYTLQYGQCGDRGQYIHFTPNFLLIDNLIIYGPRGRVFVHEWAHLRWGVFDEYNKERPFYLSRKNVVEATRCSTDITGTNVVHECQGGSCVTRKCRRDSKTGLPEPKCTFIPNKSQTARASIMFLQSLDSRRMIFYGGIKKCVLNKRQEMGLNLQSYKARVLGFSPLYFGRMVVEFCTEKTHNTEAPNLQNKICNGRSTWDVIKESADFQHAPPMRGTEAPPPPTFSLLKSRQRV

SEQ ID NO: 32 - protein, 335 aa, Rattus norvegicus
MVPVLKVLLFLTLHLLQDTKSFKVHLNNNGYEGVVIAINPSVPEDERLIPSLKEMVTQASTYLFEASQGRFYFRNVSILVPMTWKSKSEYLMPKRESYDKADVIVANSHLKYGDNPYTLQYGQCGDRGRYIHFTPNFLLTDNVRNYGPRGRVFVHEWAHLRWGVFDEYNEDRPFYISGKNTIEVTRYLCELSDSTTSYLRVFSRPYRAVQVTGCSTDIKGSKAVHERQRGSDVTRLCRWDSRTGLYEPKCKFFPDKIQTARASIMFMQNLNSVVEFCTEKTHNTEAPNLQNKICNGRSTWDVIKESADFQQAPPMRGTEAPPPPTFSLLKSRQRV

SEQ ID NO: 33 - protein, 307 aa, Rattus norvegicus
MGSLKSPVFLLVLYLLEGVLSNSLIQLNNNGYEGIVIAIDHDVPEDEALIQRIKDMVTQASPYLFEATGKRFYFKNVAILIPENWNTKPEYKRPKLETLKNADVLVSTMSPIGNDEPYTEHIGACGERGIRIHLTPDFLAGKKQTEYGPQDRTFVHEWAHFRWGVFDEYNNNEKFYLSNGKPQAVRCSATITGKHVVRRCQGGSCVTNGKCVIDRVTGLYKDNCVFIPDKNQREKASIMFNQNINSVVEFCTEKNHNKEAPNAQNQRCNLRSTWEVIQESEDFKQTTPMTAQPPAPTFSLLQTRQRI

SEQ ID NO: 34 - protein, 279 aa, Rattus norvegicus
LKLKENGYDGLLVAINPRVPEDLKLIRNIQEMITEASFYLFNATKRRVFFRSVQILIPATWTAHNYSRVKQESFDKANVLVTEQNGVPGEDPYTLQHRGCGQEGKYIHFTPNFLLNDELAAGYGSRGRVFVHEWAHLRWGVFDEYNSDKPFYVNGRNEIQVTRCSSDITGVFVCEKGLCPHEDCIISKLFREGCTFLYNSTQSATGSIMFMQSLPSVVEFCNEGTHNREAPNLQNRVCSLRSTWDVITGSSDLNHSLPVLGVELPAPPSFSLLQAGDRV

SEQ ID NO: 35 - protein, 246 aa, Rattus norvegicus
MGFSRGIVFLLLLYLLQGSDTSLVKLNENGYEDIIIAIDPAVSEDVTIIDQIKDMVTKASAYLFEATEKRFFFKNVSILIPENWTNSDQYRRPKQESYKHADIKVAPPALQGRDEPYTRQFTKCGKKAEYIHFTPDFVLGRKQKEYGDSGRLLVHEWAHLRWGVFDEYNEDQPFYSASSKKIEATRCSTGIKGMNKAQVCQGGSCITRNCRRNSTTQLYEKDCQFFPDKVQTEKSSIMFMQSIDSV

SEQ ID NO: 36 - protein, 308 aa, Rattus norvegicus
MVPVLKVLLFLTLHLLQDTKSFKVHLNNNGYEGVVIAINPSVPEDERLIPSLKEMVTQASTYLFEASQGRFYFRNVSILVPMTWKSKSEYLMPKRESYDKADVIVANSHLKYGDNPYTLQYGQCGDRGRYIHFTPNFLLTDNVRNYGPRGRVFVHEWAHLRWGVFDEYNEDRPFYISGKNTIEVTRCSTDIKGSKAVHERQRGSDVTRLCRWDSRTGLYEPKCKFFPDKIQTARASIMFMQNLNSVVEFCTEKTHNTEAPNLQNKICNGRSTWDVIKESADFQQAPPMRGTEAPPPPTFSLLKSRQRV

SEQ ID NO: 37 - protein, 162 aa, Homo sapiens
DPNVPEDETLIQQIKDMVTQASLYLFEATGKRFYFKNVAILIPETWKTKADYVRPKLETYKNADVLVAESTPPGNDEPYTEQMGNCGEKGERIHLTPDFIAGKKLAEYGPQGKAFVHEWAHLRWGVFDEYNNDEKFYLSNGRIQAVRCSAGITGTNVVKKCQ

SEQ ID NO: 38 - DNA, 31 bp, Artificial Sequence
  synthetically generated oligonucleotide
atgtcgaccatatgattcaacaaataaagga

SEQ ID NO: 39 - DNA, 33 bp, Artificial Sequence
  synthetically generated oligonucleotide
atgcggccgctcacttctttactacatttgtac

SEQ ID NO: 40 - DNA, 27 bp, Artificial Sequence
  synthetically generated oligonucleotide
catatgtcactcattcagctgaacaac

SEQ ID NO: 41 - DNA, 25 bp, Artificial Sequence
  synthetically generated oligonucleotide
catatggaagatgaaacactcattc

SEQ ID NO: 42 - DNA, 32 bp, Artificial Sequence
  synthetically generated oligonucleotide
gcggccgctcacttctttactacatttgtacc

SEQ ID NO: 43 - DNA, 30 bp, Artificial Sequence
  synthetically generated oligonucleotide
gcggccgctcacttgtttggagcttctttg

SEQ ID NO: 44 - protein, 6 aa, Artificial Sequence
  synthetically generated oligonucleotide
XKAMHX